(19) United States
(12) Patent Application Publication
(10) Pub. No.: US 2023/0104728 A1
(43) Pub. Date: Apr. 6, 2023

(54) ANTIBODY-DRUG CONJUGATE

(71) Applicant: Shenzhen Enduring Biotech, Ltd., Shenzhen (CN)

(72) Inventors: Dechun Wu, Shenzhen (CN); Shumin Liu, Shenzhen (CN); Shuqiang Yin, Shenzhen (CN); Yu Wen, Shenzhen (CN)

(73) Assignee: Shenzhen Enduring Biotech, Ltd., Shenzhen (CN)

(21) Appl. No.: 17/995,541

(22) PCT Filed: Apr. 15, 2021
(86) PCT No.: PCT/CN2021/087513

§ 371 Date: Oct. 5, 2022

(30) Foreign Application Priority Data
Apr. 15, 2020 (CN) PCT/CN2020/084880

(51) Int. Cl. A61K 47/68; A61K 47/60; A61K 47/54; A61P 35/00

(52) U.S. Cl. CPC A61K 47/6889; A61K 47/6851; A61K 47/6849; A61K 47/6805; A61K 47/6809; A61K 47/6831; A61K 47/60; A61K 47/545; A61P 35/00

(57) ABSTRACT

Provided herein is an antibody-drug conjugate (ADC) especially a PEGylated mono or bispecific antibody-drug conjugate (BsADC) prepared with site-specific conjugation to provide homogeneous conjugate with high potency and low toxicity. It also relates to a method for the preparation of the ADC, a composition comprising the ADC, and the use thereof in treating diseases.

This application claims the benefit of the filing date of the PCT application PCT/CN2020/084880 filed on Apr. 15, 2020, the entire content of which is incorporated by reference for all purpose.

### FIELD OF INVENTION

The present invention relates to an antibody-drug conjugate (ADC) especially a multi-specific antibody-drug conjugate prepared with site-specific conjugation to provide homogeneous conjugate with high potency and low toxicity. In particular, the invention relates to a long acting PEGylated mono- or bispecific single chain antibody drug conjugate prepared by site-specific conjugation of PEGylated drug conjugate to a mono- or bispecific antibody.

### BACKGROUND OF INVENTION

Cancer treatment has been progressing slowly from surgery (later 1800s) to radiation therapy (early 1900s), from chemotherapy and hormone treatment (MID 1900s) to targeted medicine (1990s), and from combination of targeted medicine with chemotherapy and hormone (early 2000s) to recent antibody drug conjugate (ADC) and the like. The concept of treating cancer with ADC can be dated back to more than 50 years ago (Decarvalho, S. et al. Nature, 1964, 202, 255-258): using antibodies as carriers to deliver extremely potent substances directly to a tumor cell. Early ADCs used non-humanized antibodies that themselves are antigenic, beta-emitting radionuclide payloads that are difficult to acquire and work with, and non-stable linkers that release cytotoxic payloads prematurely. Today's ADC technology uses humanized antibody, highly cytotoxic organic payload, and relative stable linker designed to keep the integrity of the cell-killing agent until the target is reached and the entire ADC molecule is internalized into the cell.

There are ten ADCs approved by FDA in the U.S., all of which are for cancer treatment, with more than 100 candidates of ADCs currently active in clinical trials (research report by Beacon Targeted Therapies|hansonwade). All ten approved ADCs showed severe adverse effect during treatment. As a matter of fact, 8 out of 10 approved ADCs are required to carry black box warning labels, which limit their applications in a variety of cancer indications. The biggest challenge for IgG-based ADC today is the requirement of dosing at very close to maximum tolerated dose (MTD) to show the benefit of the treatment, which results in a very narrow therapeutic window (Beck, A. et al. Nat. Rev. Drug Discov., 2017, 16, 315-337; Vankemmelbeke, M. et al. Ther. Deliv., 2016, 7, 141-144; Tolcher A. W. et al. Ann. Oncol., 2016, 27, 2168-2172). Furthermore, the toxicity profiles found for these ADCs are comparable with those of standard-of-care chemotherapeutics, with dose-limiting toxicities associated with cytotoxic warheads (Coats, S. et al. Clin. Cancer Res., 2019, 25, 5441-5448). Of approximately 80 traditional ADCs terminated in clinical trials, majority of those terminated were attributed to a poor therapeutic window or index when comparing with existing therapies. It is well documented that the site of conjugation and linker/drug hydrophobicity have significant impacts on stability, efficacy and therapeutic index of ADCs, and site-specific conjugation of a cytotoxic molecule to an antibody with hydrophilic linker can improve the therapeutic index (Junutula, J. R. et al. Nat. Biotechnol., 2008, 26, 925-932; Lyon, R. P. et al. Nat. Biotechnol., 2015, 33, 733-735). Yet many ADCs, either current in the clinical development or on the market, require cleavages of two or more interchain disulfide bonds of full-length antibodies in order to gain high DAR. Unfortunately, the approach could lead to destabilization of the protein. This is especially true for Fc bearing BsADCs because bispecific antibodies are unnatural antibodies and manufacturing of Fc bearing BsADCs with high DAR are even more difficult. Many other ADCs in the development or approved are prepared with random conjugation at either cysteine residue or lysine residues of the antibody and are heterogeneous in nature, resulting in difficulty in analysis and precise dosing in clinical setting. Moreover ADC molecules constructed from full length antibodies are considered to be too big to deep-penetrate dense solid tumor to treat mid- to late-stage cancers. Furthermore, all Fc bearing traditional ADCs have inherent toxicity due to their Fc binding to FcγRIIa on megakaryocytes (MK) and subsequent internalization followed by killing of MKs, which ultimately results in the production of platelets stopped and thrombocytopenia (Uppal, H. et al. Clin Cancer Res; 21(1) Jan. 1, 2015) and many off-target toxicities observed for antibody-drug conjugates are also driven by mannose receptor uptake, which is directly related to Fc component of the ADCs (Gorovits, B. et. al. 2013, Cancer Immunol Immunother 62, 217-223).

Therefore, there is an urgent need for a novel ADC technology with enhanced potency and improved toxic profile.

### SUMMARY OF THE INVENTION

This invention addresses the aforementioned unmet needs by providing non-immunogenic polymer modified antibody drug conjugate prepared by site-specific conjugation of polymer modified (e.g. PEGylated) drug conjugate either to an mono-specific or multi-specific antibody fragment; or to a mono-specific or multi-specific single chain antibody, with an engineered site (e.g. cysteine) for site-specific conjugation. The antibody fragment or single chain antibody can be monovalent or multivalent for the antigens.

In one aspect, the invention provides a polymer antibody drug conjugate molecule of the Formula Ia

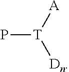

P can be a non-immunogenic polymer. T can be a multifunctional (e.g. trifunctional) small molecule linker moiety and have at least one functional group that is capable of site-specific conjugation to a mono-specific or multi-specific antibody or protein. A can be any mono-specific or multi-specific antibody or protein. D can be any cytotoxic small molecule or peptide (n≥1), and each D can be the same or different.

In particular, an aspect of the invention provides a conjugate of Formula Ib:

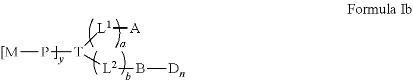

wherein

P can be a non-immunogenic polymer;

M can be H or a terminal capping group selected from C₁₋₅₀ alkyl and aryl, wherein one or more carbons of said alkyl are optionally replaced with a heteroatom;

y can be an integer selected from 1, 2, 3, 4, 5, 6, 7, 8, 9 and 10;

T can be a multi-functional linker having two or more functional groups, including but not limited to a trifunctional or tetrafunctional or any other cyclic or noncyclic multifunctional moiety (e.g. a lysine), wherein the linkage between T and (L¹)ₐ and the linkage between T and (L²)_{b} can be the same or different;

Each of L¹ and L² can be independently a bifunctional linker;

Each of a and b can be an integer selected from 0, 1, 2, 3, 4, 5, 6, 7, 8, 9 and 10;

B can be a branched linker, wherein each branch can comprise an extension spacer, a trigger unit, a self-immolating spacer or any combination of such, wherein a trigger unit can be an amino acid sequence or a trigger moiety cleavable by an enzyme, a pH liable linker that can release the drug D or its derivatives at acidic pH conditions, or a disulfide bond linker that can release the drug D or its derivative by chemical or enzymatic cleavage, or a cleavable bond that can release the drug D by certain cleavage mechanism;

A can be any mono-specific or multi-specific antibody or antigen binding protein, including an antibody fragment, a single chain antibody, a nanobody or any antigen binding fragment, which can be monovalent or multivalent for the antigens.

D can be any cytotoxic small molecule or peptide or derivative thereof and can be released from B through either enzymatic cleavage and/or self-immolating mechanism or pH induced hydrolysis with or without self-immolating mechanism; each D can be the same or different;

n can be an integer selected from 1, 2, 3, 4, 5, 6, 7, 8, 9, 10, 11, 12, 13, 14, 15, 16, 17, 18, 19, 20, 21, 22, 23, 24 and 25.

Another aspect of the invention provides a conjugate of Formula Ic:

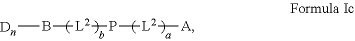

wherein each of the variables are as defined for Formula Ib.

In some embodiments, each branch of B comprises a trigger moiety, e.g. an amino acid sequence or a disulfide moiety or a β-glucoronide or β-galactoside, connected to the drug D via a self-immolating spacer or connected directly to the drug D, cleavable by e.g. cathepsins B, plasmin, matrix metalloproteinases (MMPs), glutathione, thioredoxin, thio reductase (Arunachalam, B. et. al. 2000, PNAS, 97 (2) 745-750). Examples of self-immolating spacers include but not limit to the following:

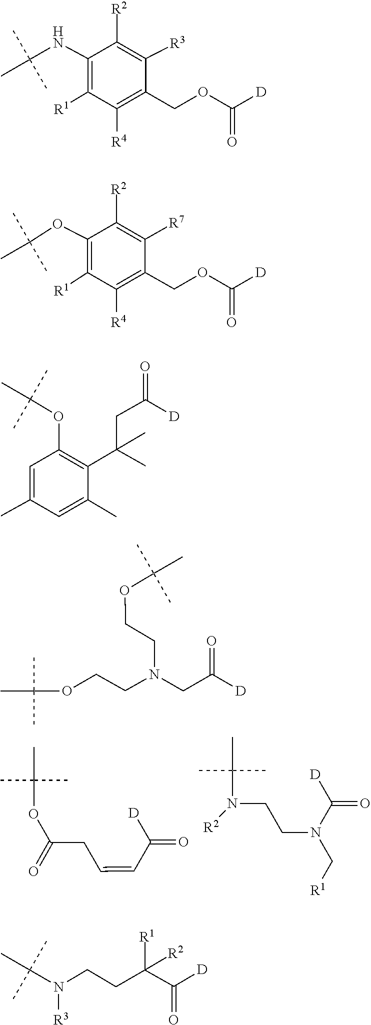

wherein R¹, R², R³, R⁴ can be H, or C₁₋₁₀ alkyl. In such embodiments, D can be any small molecule or peptide or derivative thereof containing active O or N or S functional group.

In some embodiments, each branch of B can be a pH liable linker that can release the drug D or its derivatives at acidic pH conditions at tumor site and/or inside of the tumor cell. Examples of acidic liable linkers include but not limit to the following formats:

—CR¹═N—NR¹—, —CR¹═N—O—, —CR¹═N—NR²—CO—, —N═N—CO—, —OCOO—, —NR¹—COO—.

In some embodiments, each branch of B can be a disulfide bond linker that can release the drug D or its derivatives at tumor site and/or inside of the tumor cell by chemical or enzymatic cleavage such as glutathione, thioredoxin family members (WCGH/PCK) or thio reductase.

In some embodiments, A is a mono-specific antibody that is monovalent or bivalent for the antigens, e.g. a mono-specific single chain antibody that is monovalent or bivalent for the antigens.

In some embodiments, A is a multi-specific antibody, e.g. a bispecific single chain antibody.

In some embodiments, the two binding domains of the bispecific antibody bind to two of the same tumor associated antigen (TAA) molecules, but at two different epitopes, or bind to two different TAA molecule.

In a further embodiment, A is a single chain anti-Her2×anti-Her2 antibody (SCAHer2×SCAHer2) that binds to Her2 expressed on cancer cells. The two binding domains of the SCAHer2×SCAHer2 antibody can bind to the same epitope on two Her2 molecules or to two different epitopes on two Her2 molecules. In some embodiments, the antibody has an amino acid sequence as shown in SEQ ID NO: 1 or SEQ ID NO: 2.

In some embodiments, the two binding domains of the single chain antibodies are linked via a linker, and wherein the linker can comprise a moiety such as cysteine or an unnatural amino acid residue for site-specific conjugation of the antibody to L¹.

In some embodiments, D can be selected from any DNA crosslinker agent, microtubule inhibitor, DNA alkylator, topoisomerase inhibitor or a combination thereof.

In some embodiments, D can be selected from MMAE, MMAF, SN38, DM1, DM4, calicheamycins, pyrrolobenzodiazepines, duocarmycins or a derivate thereof, or a combination thereof and the like.

In some embodiments, D is monomethyl auristatin E (MMAE), an antimitotic drug or its derivative, or SN38, a potent topoisomerase I inhibitor or its derivative or a combination thereof.

In a further embodiment, D is MMAE and is connected to a self-immolating spacer such as 4-aminobenzyl alcohol (PAB) and a trigger moiety such Valine-Citrulline.

In any of the above aspects and embodiments, the non-immunogenic polymer can be selected from the group consisting of polyethylene glycol (PEG), dextrans, carbohydrate polymers, polyalkylene oxide, polyvinyl alcohols, hydroxypropyl-methacrylamide (HPMA), and a co-polymer thereof. Preferably, the non-immunogenic polymer is PEG, such as a branched PEG or a linear PEG. The total molecule weight of the PEG can be ranged from 5000 to 100,000 Daltons, e.g., 5000 to 80,000, 10,000 to 60,000, and 20,000 to 40,000 Daltons. The PEG can be linked to the multifunctional moiety T either through a permanent bond or a cleavable bond.

Functional group for site-specific conjugation that forms linkage between (L¹)ₐ and protein A can be selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid Iodine, and the like.

In some embodiments, one of (L¹)ₐ can comprise a linkage formed from azide and alkyne or from maleimide and thiol. In some embodiments, the alkyne can be dibenzocyclooctyl (DBCO).

In some embodiments, T can be lysine, P can be PEG, and y can be 1, while the alkyne can be dibenzocyclooctyl (DBCO).

In some embodiments, A can be derived from an azide tagged mono- or multi-specific antibody or antigen binding protein including antibody fragment, a single chain antibody, a nanobody or any antigen binding fragment thereof, or a combination thereof, wherein the azide can be conjugated to an alkyne in the respective (L¹)ₐ. In other embodiments, protein A can be derived from a thiol tagged mono- or multi-specific antibody or antigen binding protein including a antibody fragment, a single chain antibody, a nanobody or any antigen binding fragment thereof, or a combination thereof, wherein the thiol can be conjugated to a maleimide in the respective (L¹)ₐ.

The above-described antibody drug conjugate can be made according to a method comprising: (i) preparing a high loading non-immunogenic polymer drug conjugate with a terminal functional group that is capable of site-specific conjugation to an antibody or a protein or its modified form; and (ii) site-specific conjugating the non-immunogenic polymer drug conjugate to an antibody or a protein or its modified structure to form a compound of Formula Ia, Ib or Ic. In some examples, the antibody or protein can be modified with a small molecule linker before the conjugation step.

The invention also provides a pharmaceutical formulation comprising the above-described antibody drug conjugate e.g. PEGylated mono- or bispecific single chain antibody drug conjugate that is monovalent or multivalent for the antigens and a pharmaceutically acceptable carrier.

The invention further provides a method of treating a disease in a subject in need thereof comprising administering an effective amount of the above-described antibody drug conjugate e.g. PEGylated mono- or bispecific single chain antibody drug conjugate that is monovalent or multivalent for the antigens.

The details of one or more embodiments of the invention are set forth in the description below. Other features, objectives, and advantages of the invention will be apparent from the drawing, description and from the claims.

The present disclosure further provides following embodiments.

Embodiment 1. A compound of the Formula (Ib)

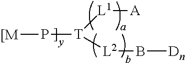

wherein

P is a non-immunogenic polymer;

M is H or a terminal capping group selected from C₁₋₅₀ alkyl and aryl, wherein one or more carbons of said alkyl are optionally replaced with a heteroatom;

y is an integer selected from 1 to 10, e.g. 1 to 5, e.g. 1, 2, 3, 4, 5, 6, 7, 8, 9 or 10;

A is an antibody or an antigen binding fragment thereof, and

T is a multifunctional small molecule linker moiety;

each of L¹ and L² is independently a hetero or homobifunctional linker;

each of a and b is an integer selected from 0-10, e.g. 0-5, e.g. 0, 1, 2, 3, 4, 5, 6, 7, 8, 9, 10;

B is a branched linker, wherein each branch has an amino acid sequence or carbohydrate moiety linked to a self-immolating spacer, wherein cleavage of the amino acid sequence or carbohydrate moiety by an enzyme triggers self-immolating mechanism to release D, or each branch has a disulfide bond or a cleavable bond, wherein cleavage of the disulfide bond or the cleavable bond releases D or its derivative;

each of D is independently a cytotoxic small molecule or peptide; and

n is an integer selected from 1-25, e.g. 1-20, 1-15, 1-10, 1-5, 5-25, 5-20, 5-15, 5-10, 10-25, 10-20, 10-15, 15-25, 15-20 or 20-25, e.g. 1, 2, 3, 4, 5, 6, 7, 8, 9, 10, 11, 12, 13, 14, 15, 16, 17, 18, 19, 20, 21, 22, 23, 24, 25.

Embodiment 2. The compound of Embodiment 1, wherein T is a tri-functional linker derived from a molecule with three functional groups independently selected from hydroxyl, amino, hydrazinyl, azide, alkene, alkyne, carboxyl (aldehyde, ketone, ester, carboxylic acid, anhydride, acyl halide), thiol, disulfide, nitrile, epoxide, imine, nitro and halide, and wherein the linkage between T and (L¹)ₐ and the linkage between T and (L²)_{b} are the same or different.

Embodiment 3. The compound of Embodiment 2, wherein T is lysine or is derived from lysine.

Embodiment 4. The compound of any of Embodiments 1-3, wherein the functional group at the linker terminal of L¹ is capable of site-specific conjugation with A, and is selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and Iodine.

Embodiment 5. The compound of any of Embodiments 1-4, wherein the antibody is a mono-specific or multi-specific full length antibody, a single chain antibody, a nanobody, or an antigen binding domain thereof.

Embodiment 6. The compound of any one of Embodiments 1-5, wherein the antibody is a mono-specific single chain antibody.

Embodiment 7. The compound of Embodiment 6, wherein the mono-specific single chain antibody binds to a tumor associated antigen (TAA) such as Her2.

Embodiment 8. The compound of Embodiment 7, wherein the mono-specific single chain antibody has two binding domains binding to Her2.

Embodiment 9. The compound of Embodiment 8, wherein the mono-specific single chain antibody has an amino acid sequence as shown in SEQ ID NO: 2.

Embodiment 10. The compound of any one of Embodiments 1-5, wherein the antibody is a bispecific antibody, e.g. a bispecific single chain antibody.

Embodiment 11. The compound of Embodiment 10, wherein the two binding domains of the bispecific antibody bind to the same tumor associated antigen (TAA), bind to two different TAAs, or bind to a TAA and an antigen expressed on T cells (e.g. a component of T cell receptor) or NK cells.

Embodiment 12. The compound of Embodiment 11, wherein the antibody is an anti-Her2×anti-Her2 single chain bispecific antibody.

Embodiment 13. The compound of Embodiment 12, wherein the antibody has an amino acid sequence as shown in SEQ ID NO: 1.

Embodiment 14. The compound of any of Embodiments 6-9, wherein the two binding domains of the mono-specific single chain antibody are linked via a linker, and wherein the linker comprises a moiety such as cysteine or an unnatural amino acid residue for site-specific conjugation of the antibody to L¹.

Embodiment 15. The compound of any of Embodiments 10-13, wherein the two binding domains of the bispecific single chain antibody are linked via a linker, and wherein the linker comprises a moiety such as cysteine or an unnatural amino acid residue for site-specific conjugation of the antibody to L¹.

Embodiment 16. The compound of any of Embodiments 14-15, wherein the unnatural amino acid is selected from genetically-encoded alkene lysines (such as N6-(hex-5-enoyl)-L-lysine), 2-Amino-8-oxononanoic acid, m or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino)hexanoic acid, azidohomoalanine, pyrrolysine analogue N6-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, NF-Acryloyl-1-lysine, Nε-5-norbornene-2-yloxycarbonyl-1-lysine, N-ε-(Cyclooct-2-yn-1-yloxy)carbonyl)-L-lysine, N-ε-(2-(Cyclooct-2-yn-1-yloxy)ethyl) carbonyl-L-lysine, genetically encoded Tetrazine Amino Acid (such as 4-(6-methyl-s-tetrazin-3-yl)aminophenylalanine).

Embodiment 17. The compound of any one of Embodiments 1-16, wherein D is selected from a DNA crosslinker agent, a microtubule inhibitor, a DNA alkylator, a topoisomerase inhibitor or a combination thereof.

Embodiment 18. The compound of Embodiments 17, wherein D is selected from MMAE, MMAF, SN38, DM1, DM4, calicheamycins, pyrrolobenzodiazepines, duocarmycins or a derivate thereof, or a combination thereof.

Embodiment 19. The compound of Embodiments 17, wherein D is selected from Vinca alkaloid, laulimalide, taxane, colchicine, tubulysins, Cryptophycins, Hemiasterlin, Cemadotin, Rhizoxin, Discodermolide, taccalonolide A or B or AF or AJ, taccalonolide AI-epoxide, CA-4, epothilone A and B, laulimalide, paclitaxel, docetaxel, doxorubicin, Camptothecin, iSGD-1882, centanamycin, PNU-159682, uncialamycin, indolinobenzodiazepine dimers, β-amanitin, Amatoxins, thailanstatins or a derivate or analogous thereof, or a combination thereof.

Embodiment 20. The compound of any one of Embodiments 1-19, wherein the non-immunogenic polymer is polyethylene glycol (PEG).

Embodiment 21. The compound of Embodiment 20 wherein the PEG is a liner PEG or a branched PEG.

Embodiment 22. The compound of any one of Embodiment 20-21, wherein at least one terminal of the polyethylene glycol is capped with methyl or a low molecule weight alkyl.

Embodiment 23. The compound of any of Embodiment 20-22, wherein a total molecule weight of the PEG is from 100 to 80000.

Embodiment 24. The compound of any one of Embodiments 20-23, wherein the PEG is linked to the trifunctional or tetrafunctional or any other cyclic or noncyclic multifunctional moiety T (e.g. a lysine) through a permanent bond or a cleavable bond.

Embodiment 25. A compound of the Formula (Ic)

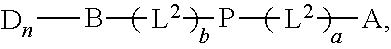

wherein

P is a liner PEG;

A is an antibody or an antigen binding fragment thereof;

each of L¹ and L² is independently a bifunctional linker;

each of a and b is an integer selected from 0-10, e.g. 0-5, e.g. 0, 1, 2, 3, 4, 5, 6, 7, 8, 9, 10;

B is a branched linker, wherein each branch has an amino acid sequence or carbohydrate moiety linked to a self-immolating spacer, wherein cleavage of the amino acid sequence or carbohydrate moiety by an enzyme triggers self-immolating mechanism to release D, or each branch has a disulfide bond or a cleavable bond, wherein cleavage of the disulfide bond or the cleavable bond releases D or its derivative;

each of D is independently a cytotoxic small molecule or peptide;

n is an integer selected from 1-25, e.g. 1-20, 1-15, 1-10, 1-5, 5-25, 5-20, 5-15, 5-10, 10-25, 10-20, 10-15, 15-25, 15-20 or 20-25, e.g. 1, 2, 3, 4, 5, 6, 7, 8, 9, 10, 11, 12, 13, 14, 15, 16, 17, 18, 19, 20, 21, 22, 23, 24, 25.

Embodiment 26. The compound of Embodiment 25, wherein the functional group at the linker terminal of L¹ is capable of site-specific conjugation with A, and is selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and Iodine.

Embodiment 27. The compound of any of Embodiments 25-26, wherein the antibody is a mono-specific or multi-specific full length antibody, a single chain antibody, a nanobody, or an antigen binding domain thereof.

Embodiment 28. The compound of Embodiment 27, wherein the antibody is a mono-specific single chain antibody, optionally wherein the mono-specific single chain antibody binds to a tumor associated antigen (TAA) such as Her2.

Embodiment 29. The compound of Embodiment 28, wherein the mono-specific single chain antibody has two binding domains binding to Her2.

Embodiment 30. The compound of Embodiment 29, wherein the mono-specific single chain antibody has an amino acid sequence as shown in SEQ ID NO: 2.

Embodiment 31. The compound of Embodiment 27, wherein the antibody is a bispecific antibody, e.g. a bispecific single chain antibody.

Embodiment 32. The compound of Embodiment 31, wherein the two binding domains of the bispecific antibody bind to the same tumor associated antigen (TAA), bind to two different TAAs, or bind to a TAA and an antigen expressed on T cells (e.g. a component of T cell receptor) or NK cells.

Embodiment 33. The compound of Embodiment 32, wherein the antibody is an anti-Her2×anti-Her2 single chain bispecific antibody.

Embodiment 34. The compound of Embodiment 33, wherein the antibody has an amino acid sequence as shown in SEQ ID NO: 1.

Embodiment 35. The compound of any of Embodiments 28-30, wherein the two binding domains of the mono-specific single chain antibody are linked via a linker, and wherein the linker comprises a moiety such as cysteine or an unnatural amino acid residue for site-specific conjugation of the antibody to L¹.

Embodiment 36. The compound of any of Embodiments 31-34, wherein the two binding domains of the bispecific single chain antibody are linked via a linker, and wherein the linker comprises a moiety such as cysteine or an unnatural amino acid residue for site-specific conjugation of the antibody to L¹.

Embodiment 37. The compound of any of Embodiments 35-36, wherein the unnatural amino acid residue for site-specific conjugation of the antibody to L¹ is selected from genetically-encoded alkene lysines (such as N6-(hex-5-enoyl)-L-lysine), 2-Amino-8-oxononanoic acid, m or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino)hexanoic acid, azidohomoalanine, pyrrolysine analogue N6-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, β-azidophenylalanine, para-azidophenylalanine, NF-Acryloyl-1-lysine, NF-5-norbornene-2-yloxycarbonyl-1-lysine, N-ε-(Cyclooct-2-yn-1-yloxy)carbonyl)-L-lysine, N-ε-(2-(Cyclooct-2-yn-1-yloxy)ethyl) carbonyl-L-lysine, genetically encoded Tetrazine Amino Acid (such as 4-(6-methyl-s-tetrazin-3-yl)aminophenylalanine).

Embodiment 38. The compound of any one of Embodiments 25-37, wherein D is selected from a DNA crosslinker agent, a Microtubule inhibitor, a DNA alkylator, a Topoisomerase inhibitor or a combination thereof.

Embodiment 39. The compound of any one of Embodiments 38, wherein D is selected from MMAE, MMAF, SN38, DM1, DM4, calicheamycins, pyrrolobenzodiazepines, duocarmycins or a derivate thereof, or a combination thereof.

Embodiment 40. The compound of any one of Embodiments 38, wherein D is selected from Vinca alkaloid, laulimalide, taxane, colchicine, tubulysins, Cryptophycins, Hemiasterlin, Cemadotin, Rhizoxin, Discodermolide, taccalonolide A or B or AF or AJ, taccalonolide AI-epoxide, CA-4, epothilone A and B, laulimalide, paclitaxel, docetaxel, doxorubicin, Camptothecin, iSGD-1882, centanamycin, PNU-159682, uncialamycin, indolinobenzodiazepine dimers β-amanitin, Amatoxins, thailanstatins or a derivate or analogous thereof, or a combination thereof.

Embodiment 41. The compound of any of Embodiment 25-40, wherein a total molecule weight of the PEG is from 100 to 80000.

Embodiment 42. The compound of any of Embodiment 1-41, wherein each of L¹ and L² is independently selected from:

—(CH₂)ₐXY(CH₂)_{b}—,

—X(CH₂)ₐO(CH₂CH₂O)_{c}(CH₂)_{b}Y—,

—(CH₂)ₐheterocyclyl-,

—(CH₂)ₐX—,

—X(CH₂)ₐY—,

—W₁—(CH₂)ₐC(O)NR₁(CH₂)_{b}O(CH₂CH₂O)_{c}(CH₂)_{d}C(O)—,

—C(O)(CH₂)ₐO(CH₂CH₂O)_{b}(CH₂)_{c}W₂C(O)(CH₂)_{d}NR₁—,

—W₃—(CH₂)ₐC(O)NR₁(CH₂)_{b}O(CH₂CH₂O)_{c}(CH₂)_{d}W₂C(O)(CH₂)ₑC(O)—,

wherein a, b, c, d and e are each an integer independently selected from 0 to 25, e.g. 0-20, 0-15, 0-10, 0-5, 5-25, 5-20, 5-15, 5-10, 10-25, 10-20, 10-15, 15-25, 15-20 or 20-25, e.g. 0, 1, 2, 3, 4, 5, 6, 7, 8, 9, 10, 11, 12, 13, 14, 15, 16, 17, 18, 19, 20, 21, 22, 23, 24, 25; each of X and Y is independently selected from C(═O), NR₁, S, O, CR₂R₃ or Null; R₁ and R₂ independently represent hydrogen, C₁₋₁₀ alkyl or (CH₂)₁₋₁₀C(═O); W₁ and/or W₃ is derived from a maleimido-based moiety and W₂ represents a triazolyl or a tetrazolyl containing group; the heterocyclyl group is selected from a maleimido-derived moiety or a tetrazolyl-based or a triazolyl-based moiety.

Embodiment 43. The compound of any of Embodiments 1-41, wherein each of (L¹)ₐ and (L²)_{b} is independently selected from:

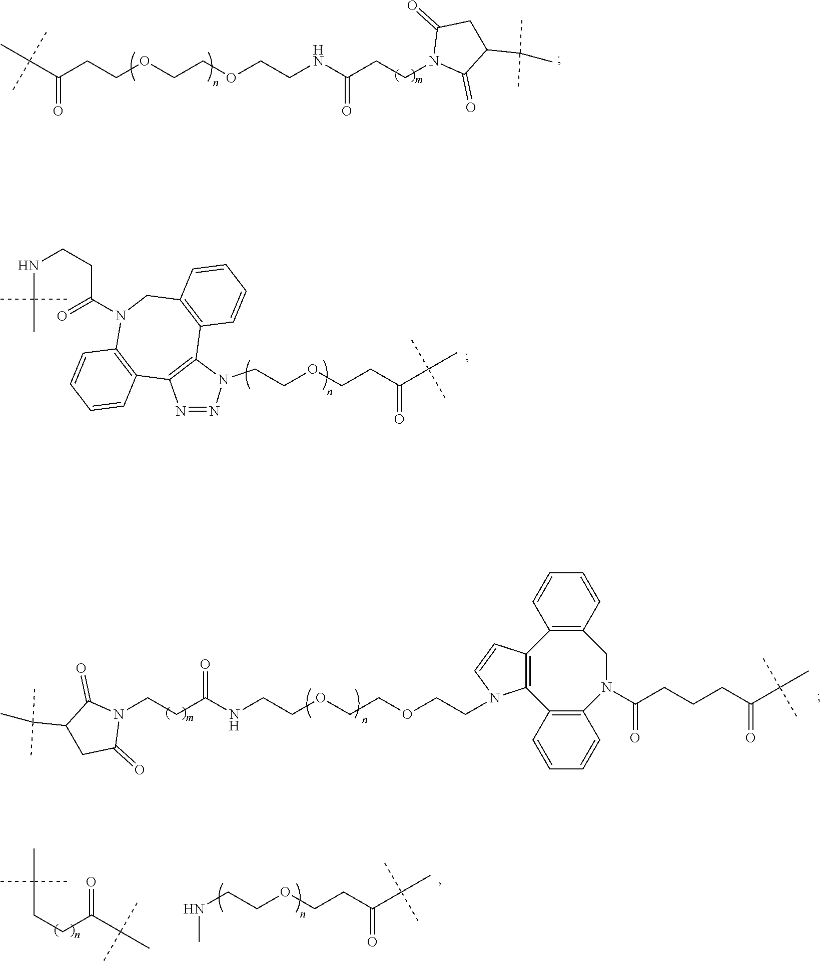

wherein n and m are integer and independently selected from 0 to 20, e.g. 0-15, 0-10, 0-5, 5-20, 5-15, 5-10, 10-20, 10-15, or 15-20, e.g. 0, 1, 2, 3, 4, 5, 6, 7, 8, 9, 10, 11, 12, 13, 14, 15, 16, 17, 18, 19, or 20.

Embodiment 44. The compound of any of Embodiment 1-43, wherein the branch linker B comprise an extension spacer, a trigger unit, a self-immolating spacer or any combination thereof, optionally wherein the trigger unit is an amino acid sequence or a β-glucoronide or β-galactoside trigger moiety cleavable by an enzyme such as cathepsin B, plasmin, matrix metalloproteinases (MMPs), β-glucuronidases, β-galactosidases; a pH liable linker that can release the drug D or its derivatives at acidic pH conditions, or a disulfide bond linker that can release the drug D or its derivatives by glutathione, thioredoxin family members (WCGH/PCK) or thio reductase.

Embodiment 45. The compound of 44, wherein the branch linker B is selected from

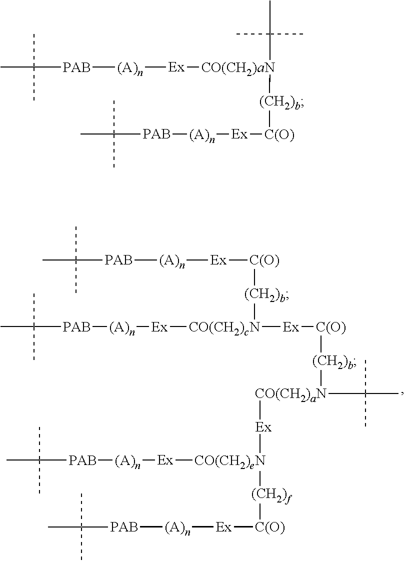

wherein:

a, b, c, d, e and f are each an integer and independently selected from 1-25 e.g. 1-20, 1-15, 1-10, 1-5, 5-25, 5-20, 5-15, 5-10, 10-25, 10-20, 10-15, 15-25, 15-20 or 20-25, e.g. 1, 2, 3, 4, 5, 6, 7, 8, 9, 10, 11, 12, 13, 14, 15, 16, 17, 18, 19, 20, 21, 22, 23, 24, 25;

(A)ₙ is a trigger unit of amino acid sequence such as Val-Cit, al-Ala, Val-Lys, Phe-Lys, Phe-Cit, Phe-Arg, Phe-Ala, Ala-Lys, Leu-Cit, Ile-Cit, Trp-Cit, D-Phe-LPhe-Lys, Phe-Phe-Lys, D-Phe-Phe-Lys, Gly-Phe-Lys, Gly-Phe-Leu-Gly, or Ala-Leu-Ala-Leu;

PAB is para-aminobenzyl alcohol;

each of Ex is an extension spacer comprising a linker chain that is independently selected from:

—NR¹(CH₂)ₓO(CH₂CH₂O)_{y}(CH₂)_{z}C(O)—,

—C(O)(CH₂)ₓR¹—,

—NR¹(CH₂)ₓO(CH₂CH₂O)_{y}(CH₂)_{z}NR²—

—NR¹(CH₂)R²—,

—NR¹(CH₂)ₓO(CH₂CH₂O)_{y}(CH₂)_{z}O—,

—O(CH₂)ₓNR¹—,

—C(O)(CH₂)ₓO—,

—O(CH₂)ₓO(CH₂CH₂O)_{y}(CH₂)_{z}C(O)—,

—C(O)(CH₂)ₓO(CH₂CH₂O)_{y}(CH₂)_{z}C(O)—,

—C(O)(CH₂)ₓC(O)—,

or Null,

wherein x, y, and z are each an integer and independently selected from 0 to 25, e.g. 0-20, 0-15, 0-10, 0-5, 5-25, 5-20, 5-15, 5-10, 10-25, 10-20, 10-15, 15-25, 15-20 or 20-25, e.g. 0, 1, 2, 3, 4, 5, 6, 7, 8, 9, 10, 11, 12, 13, 14, 15, 16, 17, 18, 19, 20, 21, 22, 23, 24, 25; and R¹ and R² independently represent hydrogen or a C₁₋₁₀ alkyl group.

Embodiment 46. The compound of any of Embodiments 1-43, wherein the branch linker B is selected from

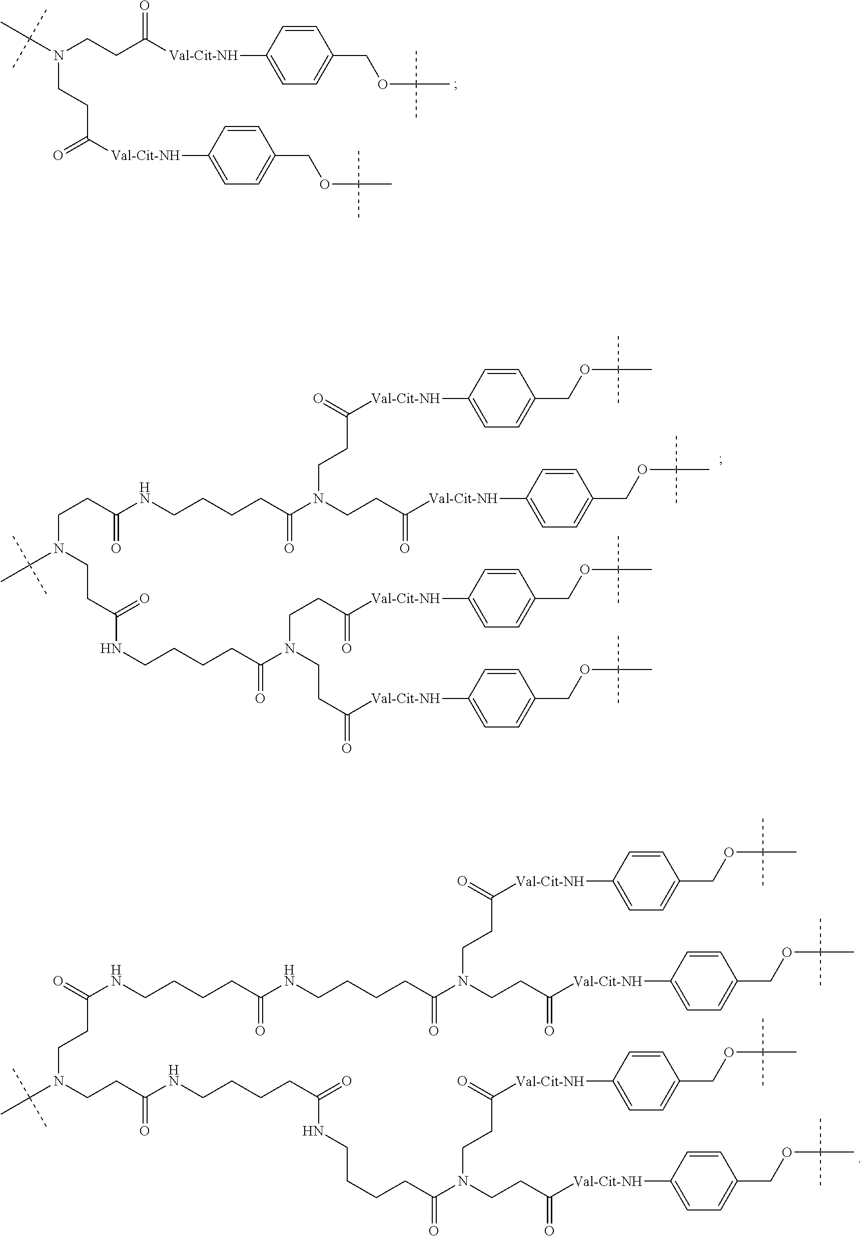

Embodiment 47. The compound of Embodiment 1 selected from the formula:

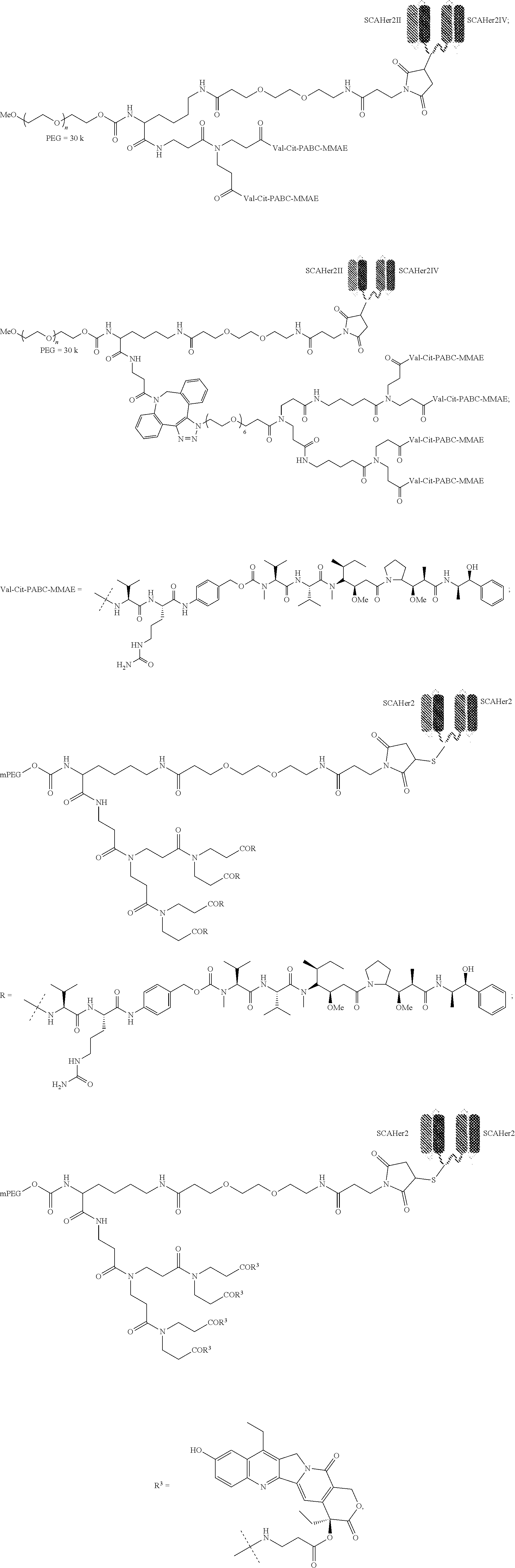

or a pharmaceutically acceptable salt thereof;

Embodiment 48. The compound of Embodiment 25 selected from the formula:

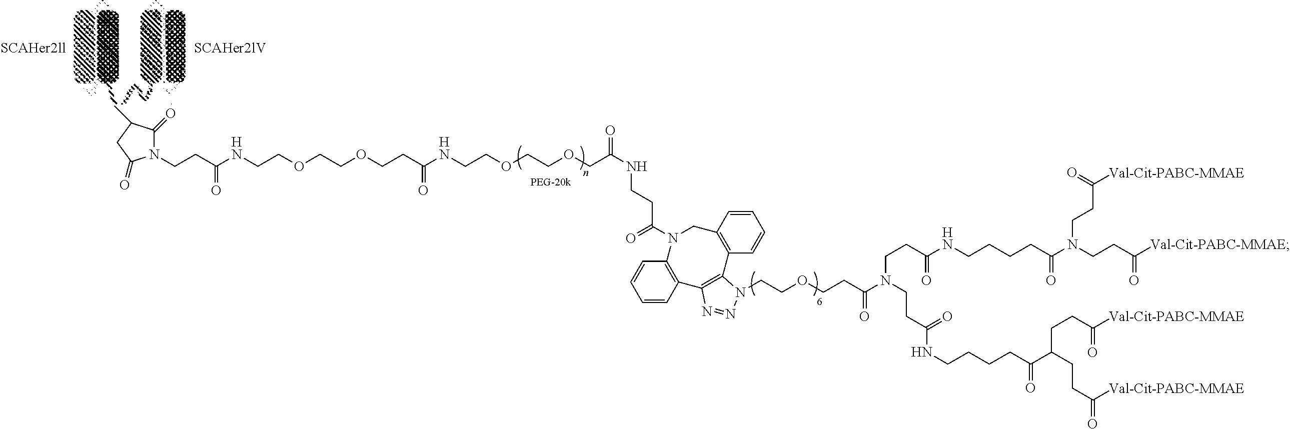

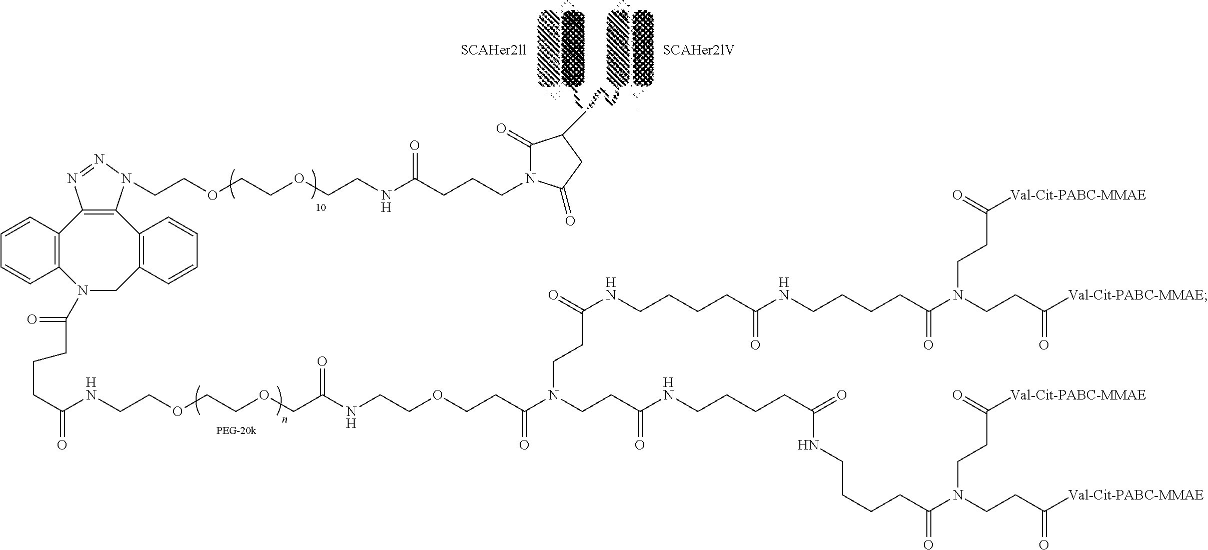

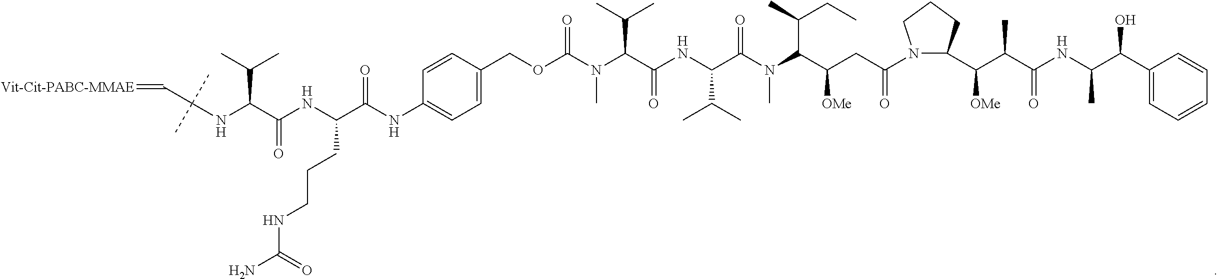

Embodiment 49. A method of preparing a compound of any one of Embodiments 1-48, comprising.

a) a step of preparation of the non-immunogenic modified (e.g. PEGylated) drug conjugate with a free functional group for site-specific conjugation;

b) a step of site-specific conjugation of the non-immunogenic modified (e.g. PEGylated) drug conjugate to an antibody to provide a compound of the Formula (Ib) or (Ic).

Embodiment 50. A pharmaceutical formulation comprising an effective amount of the compound of any one of Embodiments 1-48 and a pharmaceutically acceptable salt, carrier or excipient.

Embodiment 51. A compound of any one of Embodiments 1 to 48 for use in the treatment of a cancer selected from the group consisting of breast cancer, ovarian cancer, prostate cancer, lung cancer, pancreatic cancer, kidney cancer, bladder cancer, stomach cancer, colon cancer, colorectal cancer, salivary gland cancer, thyroid cancer and endometrial cancer; Embodiment 52. A compound of any one of Embodiments 1 to 48 for use in combination with an effective amount of another anticancer agent, immunosuppressant agent in the treatment of a cancer selected from the group consisting of breast cancer, ovarian cancer, prostate cancer, lung cancer, pancreatic cancer, kidney cancer, bladder cancer, stomach cancer, colon cancer, colorectal cancer, salivary gland cancer, thyroid cancer and endometrial cancer.

### DESCRIPTION OF THE DRAWINGS

FIG. 1 schematically illustrates a reaction scheme of preparing branched linker intermediate compound 7 described in Example 1.

FIG. 2 schematically illustrates a reaction scheme of preparing compound 14 Val-Cit-PAB-MMAE described in Example 1.

FIG. 3 schematically illustrates a reaction scheme of preparing compound 19 30 kmPEG-Lys(Mal)-(Val-Cit-PAB-MMAE)₄ described in Example 1.

FIG. 4 schematically illustrates a reaction scheme of preparing compound 20 30 kmPEG-Lys(SCAHer2/SCAHer2)-(Val-Cit-PAB-MMAE)₄ described in Example 3.

FIG. 5 schematically illustrates a reaction scheme of preparing compound 7 Val-Cit-PABC-MMAE in Example 4.

FIG. 6 schematically illustrates a reaction scheme of preparing compound 13 (branch linker B with 2×MMAE) in Example 5.

FIG. 7 schematically illustrates a reaction scheme of preparing compound 18 (branch linker B with 2×MMAE) in Example 6.

FIG. 8 schematically illustrates a reaction scheme of preparing compound 22 (branch linker B with 4×MMAE) in Example 7.

FIG. 9 schematically illustrates a reaction scheme of preparing compound 27 (branch linker B with 4×MMAE) in Example 8.

FIG. 10 schematically illustrates a reaction scheme of preparing compound 32 (30 kmPEG(Maleimide)-2MMAE) in Example 9.

FIG. 11 schematically illustrates a reaction scheme of preparing compound 35 (20 kmPEG(Maleimide)-4MMAE) in Example 10.

FIG. 12 schematically illustrates a reaction scheme of preparing compound 39 (Maleimide-20 mPEG-4MMAE) in Example 11.

FIG. 13 schematically illustrates a reaction scheme of preparing compound 41 (DBCO-20 mPEG-4MMAE) in Example 12.

FIG. 14 SDS-PAGE and SEC-HPLC analysis of purified compound 42 (SCAHer2II×SCAHer2IV) in Example 13.

FIG. 15 schematically illustrates a reaction scheme of preparing compound 43 [30 kmPEG-(SCAHer2II/SCAHer2IV)-2MMAE] and SDS-PAGE analysis in Example 14.

FIG. 16 schematically illustrates a reaction scheme of preparing compound 44 [SCAHer2II/SCAHer2IV-20 kPEG-4MMAE] and SDS-PAGE analysis in Example 15.

FIG. 17 illustrates that compound 43 (JY201) has potent in vitro cytotoxicity in Example 16.

FIG. 18 illustrates that compound 44 (JY201b) with equal payload is more potent than T-DM1 in inducing in vitro cytotoxicity to tumor cells in Example 16.

FIG. 19 illustrates that PEGylated BsADC 43 (JY201) exhibits increased internalization by target cells in Example 14.

FIG. 20 illustrates that PEGylated BsADC 43 (JY201) is retained in the target cell after internalization in Example 15.

FIG. 21 illustrates that PEGylated BsADC 43 (JY201) shows no toxicity to Megakaryocytes in Example 16.

### DETAILED DESCRIPTION OF THE INVENTION

In this invention, a PEGylated mono- or multi-specific antibody drug conjugates are provided. With this invention, there is no need to break two or more disulfide bonds of antibody to gain high DAR, and the homogeneous ADCs can be achieved, which has a significant advantage over heterogeneous ADC in terms of toxicity, efficacy, regulatory management and manufacturing, especially multi-specific ADC manufacturing.

Furthermore, this invention provides a novel structure format of PEGylated mono- or bispecific single chain antibody drug conjugate that not only shows no toxicity to megakaryocytes or other normal cells and increases therapeutic window, but also enhances the anti-tumor effect of the conjugate with increased internalization, and with relative small size of single chain antibody molecule for achieving deep penetration of solid tumor. Accordingly, this invention addresses the issues in current ADC technologies and improves cancer therapy with the novel PEGylated mono- or multi-specific single chain antibody drug conjugate.

I. Conjugate

In one aspect of the invention, compounds of formula (Ia) are provided:

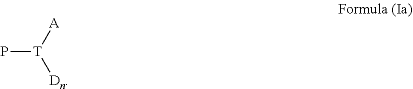

In the compound, P can be a non-immunogenic polymer. T can be a multi-functional moiety, such as a trifunctional small molecule linker moiety and have at least one functional group that is capable of site-specific conjugation with an antibody or protein. A can be any mono-specific or multi-specific antibody or protein, such as a full length antibody, a single chain antibody, a nanobody or any antigen binding fragment thereof, or a combination thereof. D can be any cytotoxic small molecule or peptide (n=1 to 25), and each D can be the same or different.

In particular, an aspect of the invention provides a conjugate of Formula Ib or Ic:

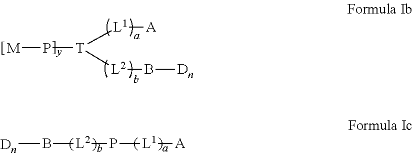

In the conjugate of Formula Ib or Formula Ic, P can be a non-immunogenic polymer such as a PEG;

M can be H or a terminal capping group selected from C₁₋₅₀ alkyl and aryl, wherein one or more carbons of said alkyl are optionally replaced with a heteroatom;

y can be an integer selected from 1, 2, 3, 4, 5, 6, 7, 8, 9, 10;

T can be a moiety having two or more functional groups, wherein the linkage between T and (L¹)ₐ and the linkage between T and (L²)_{b} can be the same or different;

Each of L¹ and L² can be independently a bifunctional linker;

Each of a and b can be an integer selected from 0, 1, 2, 3, 4, 5, 6, 7, 8, 9, 10;

B can be a branched linker, wherein each branch can comprise an extension spacer, a trigger unit, a self-immolating spacer or any combination thereof, wherein the trigger unit can be an amino acid sequence or a β-glucoronide or β-galactoside trigger moiety cleavable by an enzyme such as cathepsin B, plasmin, matrix metalloproteinases (MMPs), β-glucuronidases, or β-galactosidases; a pH liable linker that can release the drug D or its derivatives at acidic pH conditions, or a disulfide bond linker that can release the drug D or its derivatives by glutathione, thioredoxin family members (WCGH/PCK) or thio reductase.

A can be any mono-specific or multi-specific antibody or antigen binding protein including an antibody fragment, a single chain antibody, a nanobody or any antigen binding fragment, which is monovalent or multivalent for the antigens;

D can be any cytotoxic small molecule or peptide or derivative thereof and can be released from B through either enzymatic cleavage and/or self-immolating mechanism or pH induced hydrolysis; each D can be the same or different;

n can be an integer selected from 1, 2, 3, 4, 5, 6, 7, 8, 9, 10, 11, 12, 13, 14, 15, 16, 17, 18, 19, 20, 21, 22, 23, 24 and 25.

In some embodiments, each branch of B comprises a trigger moiety, e.g. an amino acid sequence or a disulfide moiety or a β-glucoronide or β-galactoside, connected to the drug D via a self-immolating spacer or connected directly to the drug D. Examples of self-immolating spacers include but not limit to the following:

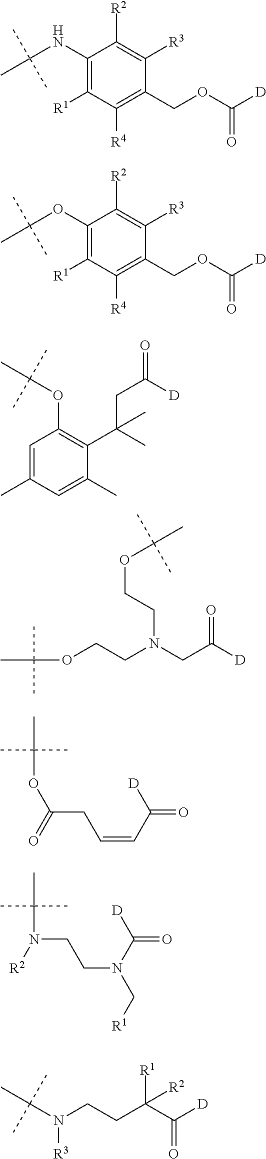

wherein R¹, R², R³, R⁴ can be H, or C₁₋₁₀ alkyl. In such embodiments, D can be any small molecule or peptide drug or derivative thereof containing active O or N or S functional group.

In some embodiments, each branch of B can comprise a pH liable linker that can release the drug D or its derivatives at acidic pH conditions at tumor site and/or inside of the tumor cell.

Examples of acidic liable linkers include but not limit to the following formats:

—CR¹═N—NR¹—, —CR¹═N—O—, —CR¹═N—NR²—CO—, —N═N—CO—, —OCOO—, —NR¹—COO—.

In some embodiments, each branch of B can comprise a disulfide bond linker that can release the drug D or its derivatives at tumor site and/or inside of the tumor cell by enzymatic cleavage, e.g. by glutathione, thioredoxin family members (WCGH/PCK) or thio reductase.

In some embodiments, A is a single chain bispecific antibody that is able to bind to two different epitopes on two Her2 antigens (SCAHer2II×SCAHer2IV).

In some embodiments, amino acid sequence of SCAHer2II×SCAHer2IV could be:

(SEQ ID NO: 1)
DIQMTQSPSSLSASVGDRVTITCKASQDVSIGVAWYQQKPGKAPKLLIY
SASYRYTGVPSRFSGSGSGTDFTLTISSLQPEDFATYYCQQYYIYPYTF
GQGTKVEIKRTGGSGGSGGSGGSGGEVQLVESGGGLVQPGGSLRLSCAA
SGFTFTDYTMDWVRQAPGKGLEWVADVNPNSGGSIYNQRFKGRFTLSVD
RSKNTLYLQMNSLRAEDTAVYYCARNLGPSFYFDYWGQGTLVTVSSGCG
SGGSGGSGGSGGEVQLVESGGGLVQPGGSLRLSCAASGFNIKDTYIHWV
RQAPGKGLEWVARIYPTNGYTRYADSVKGRFTISADTSKNTAYLQMNSL
RAEDTAVYYCSRWGGDGFYAMDYWGQGTLVTVSSGGSGGSGGSGGSGGD
IQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFG
QGTKVEIKRTHHHHHH

In some embodiments, A is a single chain anti-Her2×anti-Her2 mono-specific antibody that is able to bind to two same epitopes on two Her2 antigens.

In some embodiments, amino acid sequence of SCAHer2IV/SCAHer2IV could be:

(SEQ ID NO: 2)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTGGSGGSGGSGGSGGEVQLVESGGGLVQPGGSLRLSCAA
SGFNIKDTYIHWVRQAPGKGLEWVARIYPTNGYTRYADSVKGRFTISAD
TSKNTAYLQMNSLRAEDTAVYYCSRWGGDGFYAMDYWGQGTLVTVSSGC
GSGGSGGSGGSGGDIQMTQSPSSLSASVGDRVTITCRASQDVNTAVAWY
QQKPGKAPKLLIYSASFLYSGVPSRFSGSRSGTDFTLTISSLQPEDFAT
YYCQQHYTTPPTFGQGTKVEIKRTGGSGGSGGSGGSGGEVQLVESGGGL
VQPGGSLRLSCAASGFNIKDTYIHWVRQAPGKGLEWVARIYPTNGYTRY
ADSVKGRFTISADTSKNTAYLQMNSLRAEDTAVYYCSRWGGDGFYAMDY
WGQGTLVTVSSHHHHHH

In some embodiments, A is a single chain bispecific antibody that is able to bind to two different antigens Her2 and Her3 (SCAHer2×SCAHer3).

In some embodiments, amino acid sequence of SCAHer2IV×SCAHer3 could be:

(SEQ ID NO: 3)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTGGSGGSGGSGGSGGEVQLVESGGGLVQPGGSLRLSCAA
SGFNIKDTYIHWVRQAPGKGLEWVARIYPTNGYTRYADSVKGRFTISAD
TSKNTAYLQMNSLRAEDTAVYYCSRWGGDGFYAMDYWGQGTLVTVSSGC
GSGGSGGSGGSGGQVQLQQWGAGLLKPSETLSLTCAVYGGSFSGYYWSW
IRQPPGKGLEWIGEINHSGSTNTNPSLKSRVTISVETSKNQFSLKLSSV
TAADTAVYYCARDKWTWYFDLWGRGTLVTVSSGGSGGSGGSGGSGGDIE
MTQSPDSLAVSLGERATINCRSSQSVLYSSSNRNYLAWYQQNPGQPPKL
LIYWASTRESGVPDRFSGSGSGTDFTLTISSLQAEDVAVYYCQQYYSTP
RTFGQGTKVEIKHHHHHH

In some embodiments, A is a single chain bispecific antibody that binds to Met1 and Met2 (SCAc-Met1×SCAc-Met2).

In some embodiments, amino acid sequence of SCAc-Met1×SCAc-Met2 could be:

(SEQ ID NO: 4)
DIQMTQSPSSLSASVGDRVTITCSVSSSVSSIYLHWYQQKPGKAPKLLI
YSTSNLASGVPSRFSGSGSGTDFTLTISSLQPEDFATYYCIQYSGYPLT
FGGGTKVEIKGGSGGSGGSGGSGGQVQLVQSGAEVKKPGASVKVSCKAS
GYTFTDYYMHWVRQAPGQGLEWMGRVNPNRGGTTYNQKFEGRVTMTTDT
STSTAYMELRSLRSDDTAVYYCARTNWLDYWGQGTTVTVSGCGSGGSGG
SGGSGGQVQLVQSGAEVKKPGASVKVSCKASGYIFTAYTMHWVRQAPGQ
GLEWMGWIKPNNGLANYAQKFQGRVTMTRDTSISTAYMELSRLRSDDTA
VYYCARSEITTEFDYWGQGTLVTVSSGGSGGSGGSGGSGGDIVMTQSPD
SLAVSLGERATINCKSSESVDSYANSFLHWYQQKPGQPPKLLIYRASTR
ESGVPDRFSGSGSGTDFTLTISSLQAEDVAVYYCQQSKEDPLTFGGGTK
VEIKRHHHHHH

In some embodiments, D can be released either at tumor site or inside of tumor cells by either enzymatic and/or self-immolating mechanism or PH induced hydrolysis.

In some embodiments, D can be selected from any DNA crosslinker agent, microtubule inhibitor, DNA alkylator, topoisomerase inhibitor or a combination thereof.

In some embodiments, D can be selected from auristatins (MMAE, MMAF), Vinca alkaloid, laulimalide, taxane, colchicine, maytansines (DM1, DM4), tubulysins, Cryptophycins, Hemiasterlin, Cemadotin, Rhizoxin, Discodermolide, taccalonolide A or B or AF or AJ, taccalonolide AI-epoxide, CA-4, epothilone A and B, laulimalide, paclitaxel, docetaxel, pyrrolobenzodiazepines, duocarmycins, doxorubicin, calicheamycins, Camptothecin, SN38, iSGD-1882, centanamycin, PNU-159682, uncialamycin, indolinobenzodiazepine dimers β-amanitin, Amatoxins, thailanstatins or a derivate or analogous thereof, or a combination thereof.

In some embodiments, D is monomethyl auristatin E (MMAE), an antimitotic drug or its derivative or SN38, a potent topoisomerase I inhibitor or its derivative or a combination thereof.

In a further embodiments, D is connected to a self-immolating spacer such as 4-aminobenzyl alcohol (PAB) and a trigger moiety such Valine-Citrulline to form Val-Cit-PAB-D.

In one aspect of this invention, methods for preparing PEGylated drug conjugate that is capable of site-specific conjugating to a protein or antibody including antibody fragment or single chain mono- or multi-specific antibody are provided. In another aspect of this invention, methods for preparing PEGylated single chain BsADC are provided.

To synthesize PEGylated single-chain BsADC, coding sequence or a vector carrying a coding sequence of mono-specific single-chain antibody with valence of 1 to 5 or single-chain bispecific antibody can be synthesized and introduced into, e.g., the CHO expression systems. The proteins can be expressed and purified as described previously (WO2018075308).

For the synthesis of PEGylated drug conjugate with a side chain that has a site-specific conjugation functional group, a terminal functional group of PEG such as hydroxyl or carboxyl group and the like, can be activated and conjugated with a trifunctional small molecule moiety such as Boc or Fmoc protected lysine to form a terminal branched heterobifunctional PEG. The newly formed carboxyl group can be coupled with a branch spacer to form PEG-Lys(Boc)-B. After coupling, the protection group can be removed, and the unprotected PEGylated branch linker can be coupled with a small molecule linker that has site-specific conjugation functional group such as maleimide or DBCO to form PEG-Lys(Mal)-B or PEG-Lys(DBCO)-B. The PEGylated drug conjugate such as PEG-lys(Mal)-B-(Val-Cit-PAB-MMAE)ₙ or PEG-lys(DBCO)-B-(Val-Cit-PAB-MMAE)ₙ can be prepared by coupling reaction of PEG-Lys(Mal)-B or PEG-Lys(DBCO)-B with Val-Cit-PAB-MMAE, wherein n is an integer e.g. 2. The final step of synthesis is site-specific conjugation of PEGylated drug conjugate to a thiol or azide tagged single chain bispecific antibody.

Alternatively, for the synthesis of PEGylated drug conjugate with a side chain that has a site-specific conjugation functional group, a terminal functional group of PEG such as hydroxyl or carboxyl group and the like, can be activated and conjugated with a trifunctional small molecule moiety such as Boc or Fmoc protected lysine to form a terminal branched heterobifunctional PEG followed by removal of protection group. The PEG compound after deprotection can be coupled with a small molecule linker that has site-specific conjugation functional group such as maleimide or DBCO to form PEG-Lys(Mal)-OH or PEG-Lys(DBCO)—OH. PEG-Lys(Mal)-OH or PEG-Lys(DBCO)—OH can then be coupled with a branch moiety, of which each branch is linked with a drug D via an extension spacer, a trigger unit and/or a self-immolating spacer to form PEGylated drug conjugate such as PEG-lys(Mal)-B-(Val-Cit-PAB-MMAE)ₙ or PEG-lys(DBCO)-B-(Val-Cit-PAB-MMAE)ₙ, wherein n is an integer e.g. 2. The final step of synthesis is site-specific conjugation of PEGylated drug conjugate to a thiol or azide tagged single chain bispecific antibody to form the compound of Formula Ia. and Ib.

Alternatively, PEGylated drug conjugate can be synthesized from commercial available heterobifunctional PEG using similar procedures to form the compound of Formula Ic.

II. PEG Linker

In one embodiment of the present invention, the PEG can be of the formula:

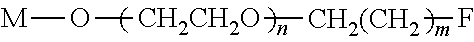

In the formula, n can be an integer from 1 to about 2300 to preferably provide a polymer having a total molecule weight of from 5000 to 40000 or greater if desired. M can be H, methyl or other low molecule weight alkyl. Non-limiting examples of M include H, methyl, ethyl, isopropyl, propyl, butyl or F₁(CH₂)_{q}CH₂. F and F₁ can be independent a terminal functional group such as hydroxyl, carboxyl, thiol, halide, amino group and the like, which is capable of being functionalized, activated and/or conjugated to a small molecule spacer or linker. q and m can be any integer from 0 to 10.

In another embodiment of present invention, the method can also be carried out with an alternative branched PEG. The branched PEG can be of the formula:

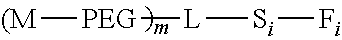

In this formula, PEG is polyethylene glycol. m can be an integer between 2 to 10 to preferably provide a branched PEG having a total molecule weight of from 5000 to 80000 or greater if desired. M can be methyl or other low molecule weight alkyl. L can be a functional linkage moiety to that two or more PEGs are attached. Non-limiting examples of such linkage moiety are: any amino acids such as glycine, alanine, lysine, or 1,3-diamino-2-propanol, triethanolamine, any 5 or 6 member aromatic ring or aliphatic rings with more than two functional groups attached. S is any non-cleavable spacer. F can be a terminal functional group such as hydroxyl, carboxyl, thiol, amino group. i is 0 or 1. When i equals to 0, the formula is shown as:

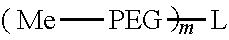

wherein: the each variables of PEG, m, M or L have the same definitions as above.

The method of the present invention can also be carried out with alternative polymeric substances such as dextrans, carbohydrate polymers, polyalkylene oxide, polyvinyl alcohols or other similar non-immunogenic polymers, the terminal groups of which are capable of being functionalized or activated. The foregoing list is merely illustrative and not intended to restrict the type of non-antigenic polymer suitable for use herein.

III. Trifunctional Linker T

T represents a trifunctional linker, connecting with P, (L¹)ₐ and (L²)_{b}. T can be derived from molecules with any combination of three functional groups, non-limiting examples of which include hydroxyl, amino, hydrazinyl, azide, alkene, alkyne, carboxyl (aldehyde, ketone, ester, carboxylic acid, anhydride, acyl halide), thiol, disulfide, nitrile, epoxide, imine, nitro and halide. The functional groups in a trifunctional linker may be the same or different. In some embodiments, one or two of the functional groups may be protected to achieve selective conjugation with other reaction partners. A variety of protecting groups are known in the art, including for example, those shown in Advanced Organic Chemistry by March (Third Edition, 1985, Wiley and Sons, New York). A functional group may also be converted into other groups before or after the reaction between T and another reaction partner. For example, a hydroxyl group may be converted into a mesylate or a tosylate group. A halide may be replaced with an azido group. An acid functional group of T may be converted to an alkyne function group by coupling with an amino group bearing a terminal alkyne.

In exemplary embodiments, T is derived from lysine, 1,3-diamino-2-propanol, or triethanolamine. One or more of the functional groups on these molecules may be protected for selective reactions. In some embodiments, T is derived from a Boc-protected lysine.

IV. Bifunctional Linker L¹ and L²

Both linkers L¹ and L² comprise linker chains that may be independently selected from —(CH₂)ₐXY(CH₂)_{b}—, —X(CH₂)ₐO(CH₂CH₂O)_{c}(CH₂)_{b}Y—, —(CH₂)ₐheterocyclyl-, —(CH₂)ₐX—, and —X(CH₂)ₐY—, wherein a, b, and c are each an integer selected from 0 to 25, with all subunits included; X or Y is independently selected from C(═O), NR₁, S, O, CR₂R₃ or Null; and R₁, R₂ and R₃ represent hydrogen, C₁₋₁₀ alkyl or (CH₂)₁₋₁₀C(═O).

The heterocyclyl linkage group within linker L¹ and L² (whether it is at internal position or at terminal position) may be derived from a maleimido-based moiety. Non-limiting examples of suitable precursors include N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), x-maleimidoundecanoic acid N-succinimidyl ester (KMUA), γ-maleimidobutyric acid N-succinimidyl ester (GMBS), ε-maleimidcaproic acid N-hydroxysuccinimide ester (EMCS), m-maleimidobenzoyl-N-hydroxysuccinimide ester(MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(β-maleimidophenyl)isocyanate (PMPI).

In some other non-limiting exemplary embodiments, each linker unit can also be derived from a haloacetyl-based moiety selected from N-succinimidyl-4-(iodoacetyl)-aminobenzoate (SIAB), N-succinimidyl iodoacetate (SIA), N-succinimidyl bromoacetate (SBA), or N-succinimidyl 3-(bromoacetamido)propionate (SBAP).

Alternatively, the heterocyclyl linkage group of the linker may be tetrazolyl or triazolyl which are formed from conjugations of different linker moieties such as DBCO and azide. Thus, the heterocyclyl group serve as a linkage point.

In some embodiments, each of (L¹)ₐ and (L²)_{b} may comprise:

—X¹—(CH₂)ₐC(O)NR(CH₂)_{b}O(CH₂CH₂O)_{c}(CH₂)_{d}C(O)— or

—C(O)(CH₂)ₐO(CH₂CH₂O)_{b}(CH₂)_{c} X²C(O)(CH₂)_{d}NR— or

—X³—(CH₂)ₐC(O)NR(CH₂)_{b}O(CH₂CH₂O)_{c}(CH₂)_{d}X²C(O)(CH₂)ₑC(O)—,

wherein X¹, X² and X³ may be the same or different and independently represent a heterocyclyl group;

a, b, c, d and e are each an integer selected from 0, 1, 2, 3, 4, 5, 6, 7, 8, 9, 10, 11, 12, 13, 14, 15, 16, 17, 18, 19, 20, 21, 22, 23, 24, 25; and

R represent hydrogen or C₁₋₁₀ alkyl.

In some embodiments, X¹ and/or X³ is derived from a maleimido-based moiety. In some embodiments, X² represents a triazolyl or a tetrazolyl containing group. In some embodiments, R represent a hydrogen. In some embodiments, a, b, c, d and e are each independently selected from 0, 1, 2, 3, 4, 5, 6, 7, 8, 9, and 10.

In some exemplary embodiments, (L¹)ₐ and (L²)_{b} can be selected from:

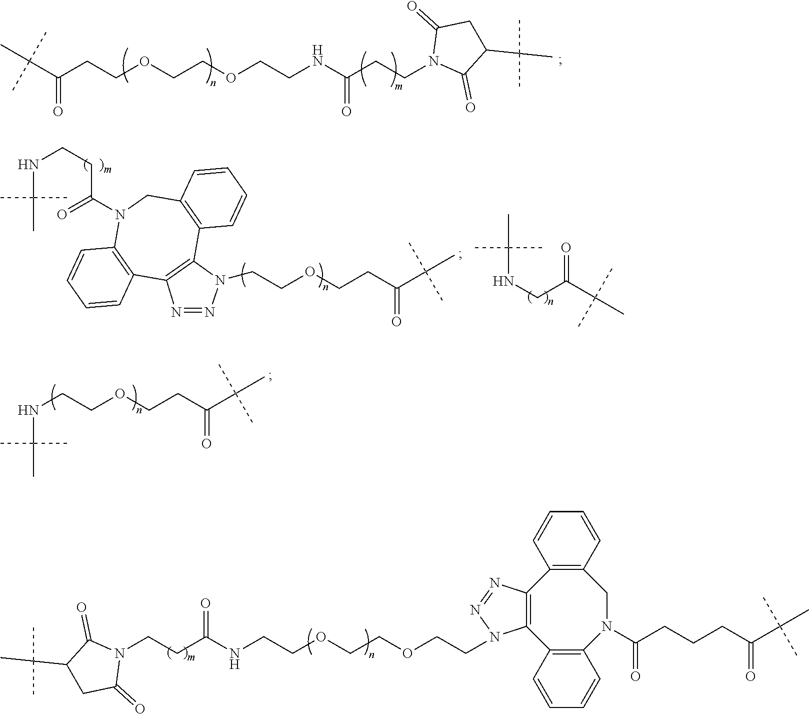

wherein n and m are integer and independently selected from 0 to 20.

V. Branched Linker B

The branched linker B can comprise a branching unite, an extension spacer, a trigger unit, a self-immolating spacer or any combination of such.

In some embodiments, a branching unite comprises structures that may be independently selected from:

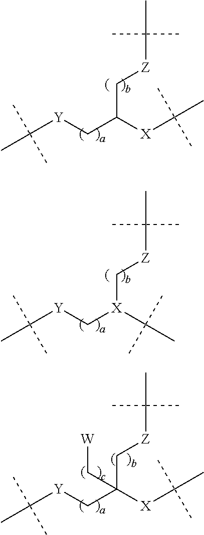

X, Y, Z, W=C(O), NR¹, NR², O, N or null
a, b, c=0-10
R¹ and R² independently represent hydrogen or C1-10 alkyl group.

In other embodiments, a branching unite comprises structures that may be independently selected from:

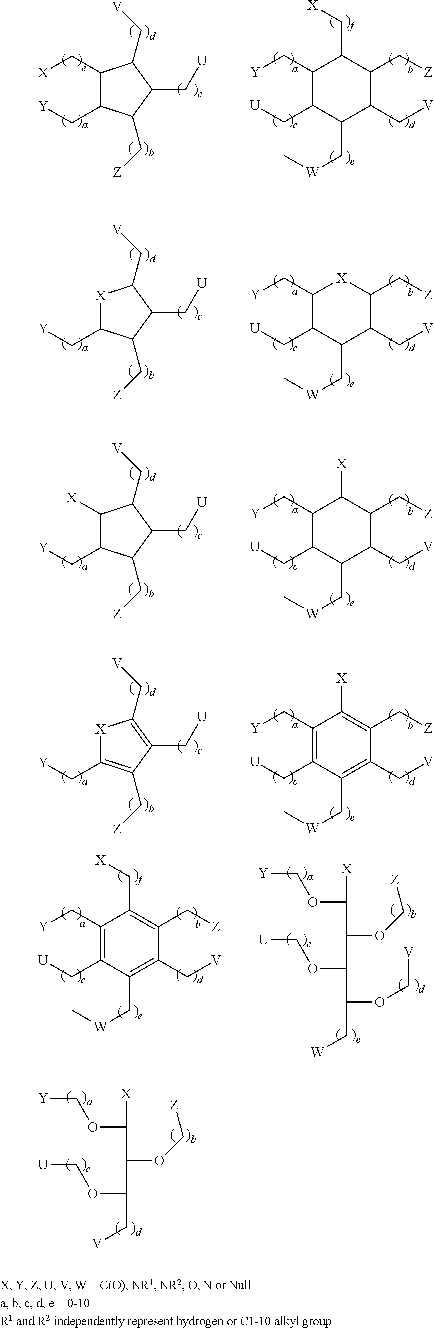

In some embodiments, an extension spacer in each branch comprises linker chains that may be independently selected from:
—X(CH₂)ₐO(CH₂CH₂O)_{b}(CH₂)_{c}Y—, —X(CH₂)ₐY—,
   or any combination thereof, wherein a, b, and c are each an integer selected from 0 to 25, all subunits included; X and Y may be selected independently from NR¹, NR², C(O), O, or Null; R¹ and R² independently represent hydrogen or C₁₋₁₀ alkyl group.

In other embodiments, a trigger unit comprises any amino acid sequence or any carbohydrate moiety or disulfide or any cleavable bond that can be enzymatically or chemically cleaved.

In some embodiments, a self-immolating spacer comprises structures that may be selected from:

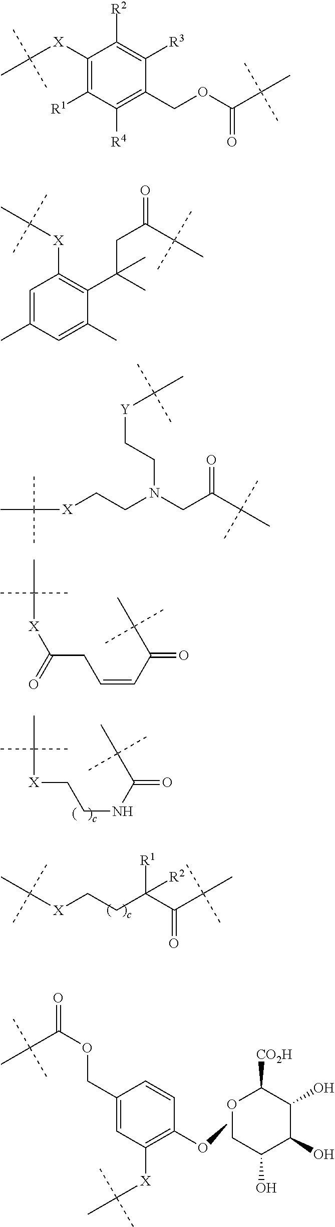

wherein R¹, R², R³ and R⁴ independently represent hydrogen or C₁₋₁₀ alkyl; X and Y can be NH or O or S, c is selected from 1 or 2.

In some embodiment, the self-immolating spacer is

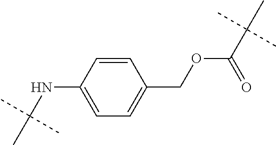

In some embodiments, the branch linker B can be selected from:

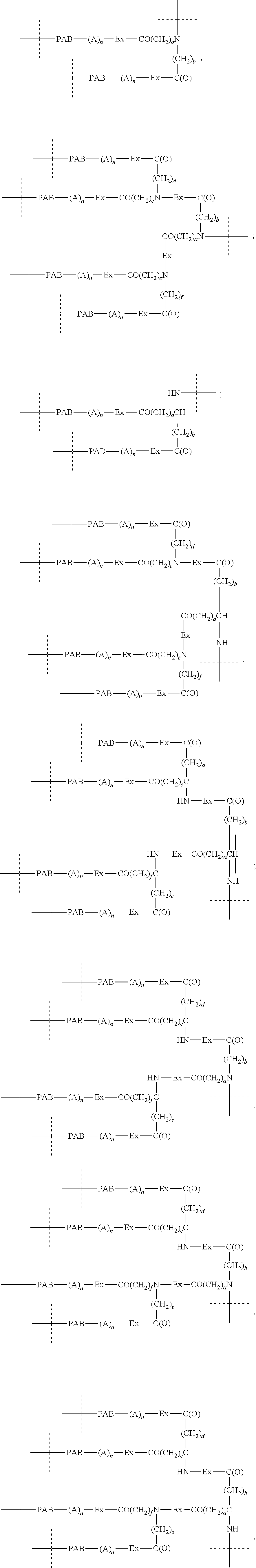

Wherein:

a, b, c, d, e and f are each an integer selected from 1-25;

(A)ₙ is a trigger unit of amino acid sequence, each A is an independent amino acid and n is any integer from 1-25;

PAB is para-aminobenzyl alcohol;

Ex is an extension spacer that comprises linker chains that may be independently selected from:

—NR¹(CH₂)ₐO(CH₂CH₂O)_{b}(CH₂)_{c}C(O)—,

—C(O)(CH₂)ₐNR—,

—NR₁(CH₂)ₐO(CH₂CH₂O)_{b}(CH₂)_{c}NR²,

—NR¹(CH₂)ₐNR²,

—NR¹(CH₂)ₐO(CH₂CH₂O)_{b}(CH₂)_{c}O—,

—O(CH₂)ₐNR¹—,

—C(O)(CH₂)ₐO—,

—O(CH₂)ₐO(CH₂CH₂O)_{b}(CH₂)_{c}C(O)—

—C(O)(CH₂)ₐO(CH₂CH₂O)_{b}(CH₂)_{c}C(O)—,

—C(O)(CH₂)ₐC(O)—,

or Null;

wherein a, b, and c are each an integer selected from 0 to 25, all subunits included; and R¹ and R² independently represent hydrogen or C₁₋₁₀ alkyl group.

In some other embodiments, the trigger unit of the amino acid sequence can be Val-Cit, al-Ala, Val-Lys, Phe-Lys, Phe-Cit, Phe-Arg, Phe-Ala, Ala-Lys, Leu-Cit, Ile-Cit, Trp-Cit, D-Phe-LPhe-Lys, Phe-Phe-Lys, D-Phe-Phe-Lys, Gly-Phe-Lys, Gly-Phe-Leu-Gly, or Ala-Leu-Ala-Leu; or their protected forms.

For preferred embodiments, the amino acid sequence can be Val-Cit, Phe-Lys, or Val-Lys.

In some exemplary embodiments, branched linker B can be selected from:

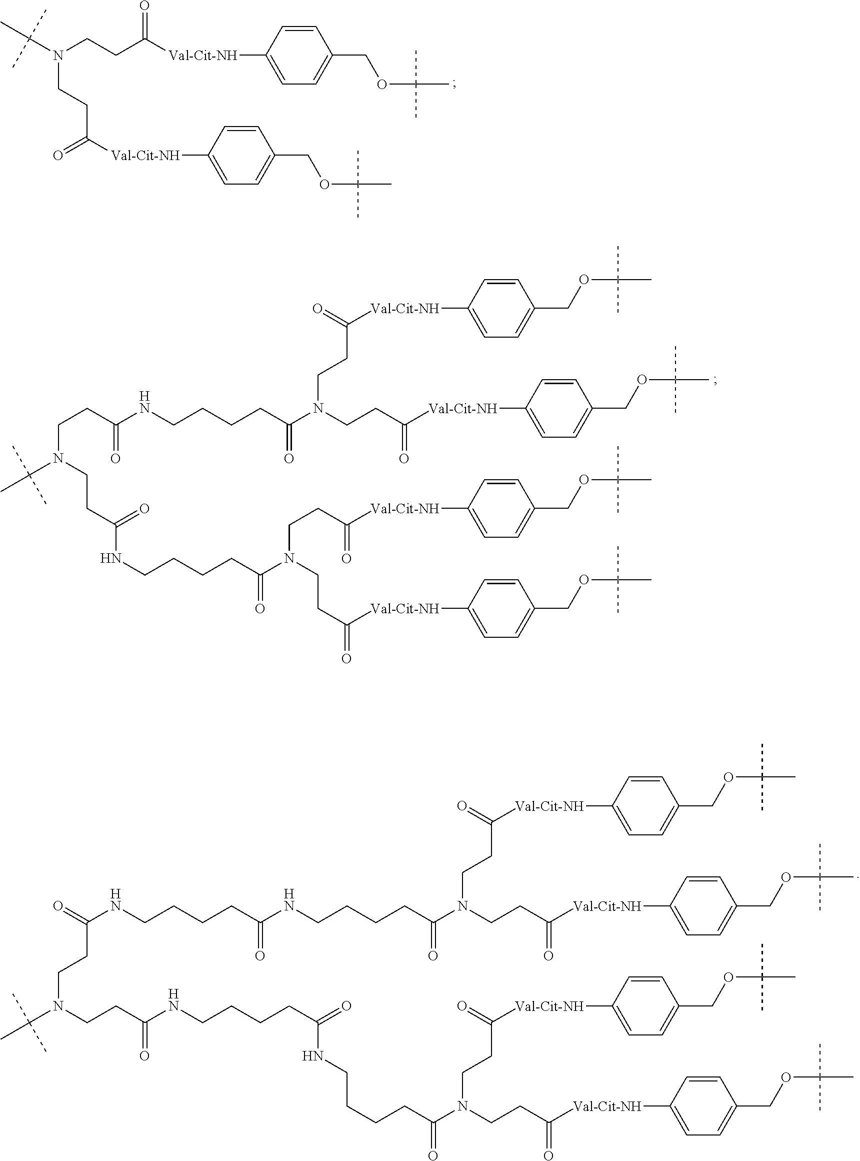

VI. Linkage Group

Different moieties of the conjugates of the present invention can be connected via various chemical linkages. Examples include but are not limited to amide, ester, disulfide, ether, amino, carbamate, hydrazine, thioether, and carbonate. For instance, the terminal hydroxyl group of a PEG moiety (P) may be activated and then coupled with lysine (T) to provide a desirable linkage point between P and T of Formula Ia or Ib. The linkage group between T and L¹ or between T and L² or between L² and B may be an amide resulting from the reaction between the amino group of a linker L² and the carboxyl group of Lysine (T) or between the carboxyl group of L¹ and the amino group of T or between the carboxyl group of L² and the amino group of B. Depending on the desirable characteristics of the conjugate, suitable linkage groups may also be incorporated between the antibody moiety (A) and the adjacent linker L¹ or between any two amino acids or between an amino acid and para-aminobenzyl alcohol.

In some embodiments, the linkage group between different moieties of the conjugates may be derived from coupling of a pair of functional groups which bear inherent chemical affinity or selectivity for each other. These types of coupling or ring formation allow for site-specific conjugation for the introduction of a protein or antibody moiety. Non-limiting examples of these functional groups that lead to site-specific conjugation include thiol, maleimide, 2′-pyridyldithio variant, aromatic or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, and triarylphosphine, boronic acid, alkyne.

VII. Cytotoxic Compound D

In some embodiments, D can include but not limit to maytansinoid (DM1, DM4) (U.S. Pat. Nos. 5,208,020; 5,416,064; EP 0425235), auristatin derivatives such as monomethyl auristatin E (MMAE) and F (MMAF) (U.S. Pat. Nos. 5,635,483; 5,780,588; 7,498,298), pyrrolobenzodiazepines, Cemadotin, SN38, Discodermolide, taccalonolide A or B or AF or AJ, taccalonolide AI-epoxide, CA-4, Vinca alkaloid, iSGD-1882, indolinobenzodiazepine dimers, uncialamycin, centanamycin, laulimalide, dolastatin, thailanstatins, Amatoxins, β-amanitin, Hemiasterlin, duocarmycins, PNU-159682, colchicine, tubulysins, calicheamicin or its derivatives thereof (U.S. Pat. Nos. 5,712,374; 5,714,586; 5,739,116; 5,767,285; 5,770,701; 5,770,710; 5,773,001; 5,877,296; Hinman, L. M. et al. Cancer Res., 1993, 53, 3336-3342; Lode, H. N. et al. Cancer Res., 1998, 58, 2925-2928), anthracycline such as daunomycin or doxorubicin (Kratz, F. et al. Curr. Med. Chem., 2006, 13, 477-523; Jeffrey, S. C. et al. Bioorg. Med. Chem. Lett., 2006, 16, 358-362; Torgov, M. Y. et al. Bioconjug. Chem., 2005, 16 717-721; Nagy, A. et al. Proc. Natl. Acad. Sci. USA, 2000, 97, 829-834; Dubowchik, G. M. et al. Bioorg. Med. Chem. Lett., 2002, 12, 1529-1532; King, H. D. et al. J. Med. Chem., 2002, 45, 4336-4343; U.S. Pat. No. 6,630,579), methotrexate, vindesine, taxanes such as docetaxel, paclitaxel, larotaxel, tesetaxel, and ortataxel, trichothecene and CC-1065.

In other embodiments, D can be an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, and enomycin.

In yet some other embodiments, D can be a radioactive atom. A variety of radioactive isotopes are available for the production of radioconjugates. Examples include At²¹¹, I¹³¹, I¹²⁵ Y⁹⁰, Re¹⁸⁶, Re¹⁸⁸, Sm¹⁵³, Bi²¹², P³², Pb²¹², and radioactive isotopes of Lu. When the radioconjugate is used for detection, it may comprise a radioactive atom for scintigraphic studies, for example Tc⁹⁹ or I¹²³, or a spin label for magnetic resonance imaging (MRI), such as I¹²³ again, I¹³¹, In¹¹¹, F¹⁹, C¹³, N¹⁵, O¹⁷, gadolinium, manganese or iron.

In some more embodiments, D can include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its synthetic analogues, adozelesin, carzelesin and bizelesin); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics, e.g., calicheamicin (Nicolaou, K. C. et al. Agnew Chem. Intl. Ed., 1994, 33, 183-186), dynemicin, esperamicin, as well as neocarzinostatin chromophore and related chromoprotein enediyne antibiotic chromomophores, aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, e.g. paclitaxel (TAXOL®) and doxetaxel (TAXOTERE®); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor rubitecan (9-nitrocamptothecin or RFS-2000); difluoromethylornithine; retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, 4-hydroxytamoxifen, trioxifene, keoxifene, LY117018, onapristone, and toremifene; and anti-androgens such as flutamide, nilutamide, bicalutamide, leuprolide, and goserelin; and pharmaceutically acceptable salts, acids or derivatives of any of the above.

VIII. Antibody and Target

A number of therapeutic antibodies directed against cell surface molecules and/or their ligands are known. These antibodies can be used for the selection and construction of tailor-made specific recognition binding moiety in the mono- or multi-specific ADC. Examples include Blinatumomab/BLINCYTO (CD3/CD19), Rituxan/MabThera/Rituximab (CD20), H7/Ocrelizumab (CD20), Zevalin/Ibrizumomab (CD20), Arzerra/Ofatumumab (CD20), HLL2/Epratuzumab, Inotuzomab (CD22), Zenapax/Daclizumab, Simulect/Basiliximab (CD25), Herceptin/Trastuzumab, Pertuzumab (Her2/ERBB2), Mylotarg/Gemtuzumab (CD33), Raptiva/Efalizumab (Cd11a), Erbitux/Cetuximab (EGFR, epidermal growth factor receptor), IMC-1121B (VEGF receptor 2), Tysabri/Natalizumab (α4-subunit of α4β1 and α4β7 integrins), ReoPro/Abciximab (gpIIb-gpIIa and αvβ-integrin), Orthoclone OKT3/Muromonab-CD3 (CD3), Benlysta/Belimumab (BAFF), Tolerx/Oteliximab (CD3), Soliris/Eculizumab (C5 complement protein), Actemra/Tocilizumab (IL-6R), Panorex/Edrecolomab (EpCAM, epithelial cell adhesion molecule), CEA-CAM5/Labetuzumab (CD66/CEA, carcinoembryonic antigen), CT-11 (PD-1, programmed death-1 T-cell inhibitory receptor, CD-d279), H224G11 (c-Met receptor), SAR3419 (CD19), IMC-A12/Cixutumumab (IGF-1R, insulin-like growth factor 1 receptor), MEDI-575 (PDGF-R, platelet-derived growth factor receptor), CP-675, 206/Tremelimumab (cytotoxic T lymphocyte antigen 4), RO5323441 (placenta growth factor or PGF), HGS1012/Mapatumumab (TRAIL-R1), SGN-70 (CD70), Vedotin (SGN-35)/Brentuximab (CD30), and ARH460-16-2 (CD44).

The mono- or multi-specific ADC disclosed herein can be used in the preparation of medicaments for the treatment of an oncologic disease, a cardiovascular disease, an infectious disease, an inflammatory disease, an autoimmune disease, a metabolic (e.g., endocrine) disease, or a neurological (e.g., neurodegenerative) disease. Exemplary non-limiting examples of these diseases are Alzheimer's disease, non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, carcinomas (such as carcinomas of the oral cavity, gastrointestinal tract, colon, stomach, pulmonary tract, lung, breast, ovary, prostate, uterus, endometrium, cervix, urinary bladder, pancreas, bone, liver, gall bladder, kidney, skin, and testes), melanomas, sarcomas, gliomas, and skin cancers, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, psoriasis, or fibrosing alveolitis.

A number of cell surface markers and their ligands are known. For example cancer cells have been reported to express at least one of the following cell surface markers and/or ligands, including but not limited to, carbonic anhydrase IX, α-fetoprotein, α-actinin-4, A3 (antigen specific for A33 antibody), ART-4, B7, Ba-733, BAGE, BrE3-antigen, CA125, CAMEL, CAP-1, CASP-8/m, CCCL19, CCCL21, CD1, CD1a, CD2, CD3, CD4, CDS, CD8, CD1-1A, CD14, CD15, CD16, CD18, CD19, CD20, CD21, CD22, CD23, CD25, CD29, CD30, CD32b, CD33, CD37, CD38, CD40, CD40L, CD45, CD46, CD54, CD55, CD59, CD64, CD66α-e, CD67, CD70, CD74, CD79a, CD80, CD83, CD95, CD126, CD133, CD138, CD147, CD154, CDC27, CDK-4/m, CDKN2A, CXCR4, CXCR7, CXCL12, HIF-1-α, colon-specific antigen-p (CSAp), CEA (CEACAM5), CEACAM6, c-met, DAM, EGFR, EGFRvIII, EGP-1, EGP-2, ELF2-M, Ep-CAM, Flt-1, Flt-3, folate receptor, G250 antigen, GAGE, GROB, HLA-DR, HM1.24, human chorionic gonadotropin (HCG) and its subunits, HER2/neu, HMGB-1, hypoxia inducible factor (HIF-1), HSP70-2M, HST-2 or 1a, IGF-1R, IFN-7, IFN-α, IFN-β, IL-2, IL-4R, IL-6R, IL-13R, IL-15R, IL-17R, IL-18R, IL-6, IL-8, IL-12, IL-15, IL-17, IL-18, IL-25, insulin-like growth factor-1 (IGF-1), KC4-antigen, KS-1-antigen, KS 1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5, MUM-1/2, MUM-3, NCA66, NCA95, NCA90, pancreatic cancer mucin, placental growth factor, p53, PLAGL2, prostatic acid phosphatase, PSA, PRAME, PSMA, P1GF, ILGF, ILGF-1R, IL-6, IL-25, RS5, RANTES, T101, SAGE, 5100, survivin, survivin-2B, TAC, TAG-72, tenascin, TRAIL receptors, TNF-α, Tn-antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, c-Met, an oncogene marker and an oncogene product (Sensi, M. et al. Clin. Cancer Res., 2006, 12, 5023-5032; Parmiani, G. et al. J. Immunol., 2007, 178, 1975-1979; Castelli, C. et al. Cancer Immunol. Immunother., 2005, 54, 187-207). Thus, antibodies recognizing such specific cell surface receptors or their ligands can be used for specific and selective recognition binding moieties in the mono- or multi-specific ADC of this invention, targeting and binding to a number of cell surface markers or ligands that are associated with a disease.

In some embodiments, for the treatment of cancer/tumors, mono- or multi-specific ADCs are used to target tumor-associated antigens (TAAs), such as those reported in Herberman, “Immunodiagnosis of Cancer”, in Fleisher ed., “The Clinical Biochemistry of Cancer”, page 347 (American Association of Clinical Chemists, 1979) and in U.S. Pat. Nos. 4,150,149; 4,361,544; 4,444,744.

Reports on tumor associated antigens include Mizukami et al., Nature Med. 2005 11, 992-997; Hatfield et al., Curr. Cancer Drug Targets 2005, 5229-248; Vallbohmer et al., J. Clin. Oncol. 2005, 23, 3536-3544; and Ren et al., Ann. Surg. 2005, 242, 55-63, each incorporated herein by reference with respect to the TAAs identified. Where the disease involves a lymphoma, leukemia or autoimmune disorder, targeted antigens may be selected from the group consisting of CD4, CD5, CD8, CD14, CD15, CD19, CD20, CD21, CD22, CD23, CD25, CD33, CD37, CD38, CD40, CD40L, CD46, CD54, CD67, CD74, CD79a, CD80, CD126, CD138, CD154, CXCR4, B7, MUC1 or 1a, HM1.24, HLA-DR, tenascin, VEGF, P1GF, ED-B fibronectin, an oncogene, an oncogene product (e.g., c-Met or PLAGL2), CD66α-d, necrosis antigens, IL-2, T101, TAG, IL-6, MIF, TRAIL-R1 (DR4) and TRAIL-R₂ (DR5).

Antibodies against the above-mentioned antigens can be used as the binding domain or moieties to make ADCs or BsADCs of this invention. Various BsADCs can be made against two different targets.

Examples of the antigen pairs include CD19/CD3, BCMA/CD3, different antigens of the HER family in combination (EGFR, HER2, HER3), IL17RA/IL7R, IL-6/IL-23, IL-1-β/IL-8, IL-6 or IL-6R/IL-21 or IL-21R, ANG2/VEGF, VEGF/PDGFR-β, VEGF 2/CD3, PSMA/CD3, EPCAM/CD3, combinations of antigens selected from a group consisting of VEGFR-1, VEGFR-2, VEGFR-3, FLT3, c-FMS/CSF1R, RET, c-Met, EGFR, Her2/neu, HER3, HER4, IGFR, PDGFR, c-KIT, BCR, integrin and MMPs with a water-soluble ligand is selected from the group consisting of VEGF, EGF, PIGF, PDGF, HGF, and angiopoietin, ERBB-3cC-Met, ERBB-2/c-Met, EGF receptor 1/CD3, EGFR/HER3, PSCA/CD3, c-Met/CD3, ENDOSIALIN/CD3, EPCAM/CD3, IGF-1R/CD3, FAPALPHA/CD3, EGFR/IGF-1R, IL 17A/F, EGF receptor 1/CD3, and CD19/CD16. Additional examples of bispecific ADCs can have (i) a first specificity directed to a glycoepitope of an antigen selected from the group consisting of Lewis x-, Lewis b- and Lewis γ-structures, Globo H-structures, KH1, Tn-antigen, TF-antigen and carbohydrate structures of Mucins, CD44, glycolipids and glycosphingolipids, such as Gg3, Gb3, GD3, GD2, Gb5, Gm1, Gm2, and sialyltetraosylceramide and (ii) a second specificity directed to an ErbB receptor tyrosine kinase selected from the group consisting of EGFR, HER2, HER3 and HER4. GD2 in combination with a second antigen binding site is associated with an immunological cell chosen from the group consisting of T-lymphocytes NK cell, B-lymphocytes, dendritic cells, monocytes, macrophages, neutrophils, mesenchymal stem cells, neural stem cells.

A monospecific or bispecific antibody can be joined together with another monospecific or bispecific antibody using the method disclosed herein to make multi-specific ADCs. By using already available monospecific or bispecific therapeutic binding entities, such as those therapeutic antibodies described above, a fast and easy production of the required multi-specific binding molecule can be achieved. With this tailor-made generation of multi-specific ADC by combining two or more single therapeutic molecules for simultaneous targeting and binding to two or more different epitopes, an additive/synergistic effect can be expected in comparison to the single targeting ADC.

In some embodiments, multi-specific ADCs of this invention are made using antibody pairs that specifically interact and show measurable affinities to the following target pairs.

| Single chain | | |
|---|---|---|
| antibody | | |
| fragments | | Diseases (or |
| Targets | Mechanisms of action | healthy volunteers) |
| CD3, EpCAM | Retargeting of T cells to | Malignant ascites in |
| | tumor, Fc mediated | EpCAM positive tumors, |
| | effector functions | Solid tumor |
| CD3, Her2 | Retargeting of T cells to | Metastatic breast cancer, |
| | tumor | Advanced solid tumors |
| CD3, CD19 | Retargeting of T cells to | Precursor B-cell ALL, |
| | tumor | ALL, DLBCL, NHL |
| CD3, CEA | Retargeting of T cells to | Gastrointestinal |
| | tumor | adenocancinoma |
| CD3, PSMA | Retargeting of T cells to | Prostate cancer |
| | tumor | |
| CD3, CD123 | Retargeting of T cells to | AML |
| | tumor | |
| CD3, gpA33 | Retargeting of T cells to | Colorectal cancer |
| | tumor | |
| CD30, CD16A | Retargeting of NK cells | Hodgkin's Lymphoma |
| | to tumor cells | |
| CD3, GD2 | Retargeting of T cells to | Neuroblastoma and |
| | tumor | osteosarcoma |
| CD3, EGFR | Autologous activated | Lung and other solid |
| | T cells to EGFR-positive | tumors, |
| | tumor | Colon and pancreatic |
| | | cancers |
| CD28, MAPG | Retargeting of T cells to | Metastatic melanoma |
| | tumor | |
| CD3, peptide | Retargeting of T cells to | Metastatic melanoma |
| MHC | tumor | |
| CD19, CD22 | Targeting of protein | B cell leukemiaor |
| | toxin to tumor | lymphoma |
| EGFR HER3 | Blockade of 2 receptors, | Head and neck cancer |
| | ADCC | Colorectal cancer |
| EGFR, c-Met | Blockade of 2 receptors | Advanced or metastatic |
| | | cancer |
| HER2, HER2 | Blockade of 2 same or | Gastric and esophageal |
| | different receptors | cancers Breast cancer |
| HER2, HER3 | Blockade of 2 receptors | Gastric and esophageal |
| | | cancers Breast cancer |
| IGF-1R, HER3 | Blockade of 2 receptors | Advanced solid tumors |
| Ang2, VEGFA | Blockade of 2 | Solid tumors, Wet AMD |
| | proangiogenics | |
| CEA, HSG | Pretargeting tumor for | Colorectal<comma> |
| | PET or radio imaging | breast and lung cancers |
| IL-1α, IL-1β | Blockade of 2 | Osteoarthritis |
| | proinflammatory cytokines | |
| TNF, IL-17A | Blockade of 2 | Rheumatoid arthritis, |
| | proinflammatory cytokines | Plaque psoriasis |
| IL-13, IL-4 | Blockade of 2 | Idiopathic pulmonary |
| | proinflammatory cytokines | fibrosis |
| IL-13 IL-4 | Blockade of 2 | (Healthy volunteers) |
| | proinflammatory cytokines | |
| TNF, HSA | Blockade of | Rheumatoid arthritis |
| | proinflammatory cytokine, | |
| | binds to HSA to increase | |
| | half-life | |
| IL-17A/F, | Blockade of 2 | (Healthy volunteers) |
| HSA | proinflammatory cytokines, | |
| | binds to HSA to increase | |
| | half-life | |
| IL-6R, HSA | Blockade of | Rheumatoid arthritis |
| | proinflammatory cytokine, | |
| | binds to HSA to increase | |
| | half-life | |
| RANKL, HSA | Blockade of bone | Postmenopausal bone |
| | resorption, binds to HSA | loss |
| | to increase half-life | |
| Factor Ixa, | Plasma coagulation | Hemophilia |
| factor X | | |

In some embodiment, a BsADC comprises a bispecific single chain antibody, wherein the two binding domains of the bispecific single chain antibody are linked via a linker. In some embodiments, the linker comprises a moiety such as cysteine or an unnatural amino acid residue that can be used for site-specific conjugation of the antibody to a non-immunogenic polymer drug conjugate, e.g. PEGylated drug conjugate. In some other embodiments, one or both of the two binding domains of the bispecific single chain antibody comprises a cysteine or an unnatural amino acid residue that can be used for site-specific conjugation of the antibody to a non-immunogenic polymer drug conjugate, e.g. PEGylated drug conjugate.

In a preferred embodiment, a BsADC is a conjugate of two antibodies or antigen-binding fragments (such as Fabs, scFvs, and the like) thereof that specifically interact and show measurable affinities to two different epitopes of Her2.

IX. Synthesis

Once the desired size and number of branches of PEG have been selected, the terminal functional group of PEG such as hydroxyl, carboxyl group and the like can be converted to terminal branched heterobifunctional groups using any art-recognized process (WO2018075308). Broadly stated, the terminal branched heterobifunctional PEG can be prepared by activating terminal hydroxyl or carboxyl group of the PEG with N-Hydroxysuccinimide using reagents such as Di(N-succinimidyl) carbonate (DSC), triphosgene and the like in the case of terminal hydroxyl group or using coupling reagents such as N,N-Diisopropylcarbodiimide (DIPC), 1-Ethyl-3-(3-dimethylaminopropyl)carbodiimide (EDC) and the like in the case of terminal carboxyl group in the presence of base such as 4-Dimethylaminopyridine (DMAP), pyridine and the like to form activated PEG.

Next, the activated PEG can be reacted with a trifunctional small molecule such as lysine derivative H-Lys(Boc)-OH in the presence of base such as Diisopropylamine (DIPEA) to form a terminal branched heterobifunctional PEG with a free carboxyl group and a Boc-protected amino group PEG-Lys(Boc)-COOH. As will be appreciated by those of ordinary skill, other terminal functional groups of PEG such as halide, amino, thiol group and the like and other trifunctional small molecules containing any combination of three functional groups from the list of —NH₂, —NHNH₂, —COOH, —OH, —C(O)X (X=halides), —N═C═O, —SH, anhydride, halides, maleimido, C═C, C≡C and the like or their protected version can be used as alternatives for the same purpose if desired.

Removal of Boc by TFA followed by reaction with a maleimide tagged spacer such as NHS-PEG₂-Maleimide produces PEG-Lys(Mal)-COOH.

Separately, the cytotoxic drug (e.g. MMAE) linked with a trigger (e.g. val-cit) and a self-immolating spacer (e.g. PABC) is coupled to a branch unit with the coupling reagent such as EDC/HOBT to generate B-D: e.g.

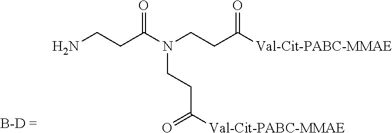

Target product could be formed by coupling PEG-Lys(Mal)-COOH with B-D with coupling reagent such as DCC to form PEGylated drug conjugate PEG-Lys(Mal)-(Val-Cit-PAB-MMAE)₂.

Monospecific antibodies that is bivalent for the antigens or Bispecific antibodies such as SCAHer2II×SCAHer2IV can be prepared through genetic manipulation of expression systems. For example, DNA encoding a bispecific scFv can be synthesized and introduced into an expression system (e.g, CHO cells). The protein of interest is then expressed and purified through chromatography technologies.

To prepare a PEGylated single chain ADC that is bivalent for the antigens or BsADC, the PEGylated drug conjugate with functional group maleimide or DBCO can be reacted site specifically with free thiol or azide functional group of a bifunctional antibody such as SCAHer2IV×SCAHer2IV or SCAHer2II×SCAHer2IV that is either genetically inserted or through derivatization, to form PEG-Lys(SCAHer2IV×SCAHer2IV)-(Val-Cit-PAB-MMAE)₂ or PEG-Lys(SCAHer2II×SCAHer2IV)-(Val-Cit-PAB-MMAE)₂.

PEGylated multi-specific antibody can be prepared similarly using multi-specific antibody instead of mono- or bispecific antibody.

In addition to thiol/maleimide or DBCO/azide site-specific conjugation group pair exemplified in this invention, as will be appreciated by those of ordinary skill, other known pairs of site-specific conjugation groups, such as trans-cyclooctenes/tetrazines pair; carbonyl/hydrazide; carbonyl/oxime; Suzuki-Miyaura Cross-Coupling reagent pair; Sonogashira Cross-Coupling reagent pair; Staudinger Ligation reagent pair; Knoevenagel-Intra Michael addition reagent pair, active amine/acrylate pair and the like can be similarly designed and used as alternatives for the same purpose if desired. The foregoing list of site-specific conjugation group pairs is merely illustrative and not intended to restrict the type of site-specific conjugation group pairs suitable for use herein.

X. Compositions

The present invention also provides a composition, e.g., a pharmaceutical composition, containing the compound of the present invention, formulated together with a pharmaceutically acceptable carrier. For example, a pharmaceutical composition of the invention can comprise a compound (e.g. a bispecific antibody-drug conjugate) that binds to two different of epitopes of Her2 receptor.

Therapeutic formulations of this invention can be prepared by mixing the mono- or multi-specific molecule drug conjugate having the desired degree of purity with optional physiologically acceptable carriers, excipients or stabilizers in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 amino acid residues) proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween, Pluronics, or PEG.

The formulation may also contain more than one active compound as necessary for the particular indication being treated, preferably those with complementary activities that do not adversely affect each other. For instance, the formulation may further comprise another antibody or multi-specific antibody, cytotoxic agent, chemotherapeutic agent or ADC. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980)._

Sustained-release preparations may be prepared. Suitable examples of sustained-release preparations include semipermeable matrices of solid hydrophobic polymers containing the mono- or multi-specific molecules, which matrices are in the form of shaped articles, e.g., films, or microcapsule. Examples of sustained-releasable matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the Lupron Depot (injectable microspheres composed oflactic acid-glycolic acid copolymer and leuprolide acetate), and poly-d(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Pharmaceutical compositions of the invention can be administered in combination therapy, i.e., combined with other agents. Examples of therapeutic agents that can be used in combination therapy are described in greater detail below.

The formulations to be used for in vivo administration must be sterile. This can be readily accomplished by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

XI. Dosage

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the mono- or multi-specific molecule drug conjugate of this invention, the dosage ranges from about 0.0001 to 100 mg/kg, and more usually 0.01 to 50 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration daily, twice per week, once per week, once every two weeks, once every three weeks, once every four weeks, once a month, once every 3 months or once every three to 6 months. Preferred dosage regimens for mono- or multi-specific drug conjugate of the invention include 1 mg/kg body weight or 3 mg/kg body weight via intravenous administration, with the mono- or multi-specific drug conjugate being given using one of the following dosing schedules: (i) every four weeks for six dosages, then every three months; (ii) every three weeks; (iii) 3 mg/kg body weight once followed by 1 mg/kg body weight every three weeks.

Alternatively, mono- or multi-specific drug conjugate can be administered as a sustained release formulation, in which case less frequent administration is required. Dosage and frequency vary depending on the half-life of the mono- or multi-specific drug conjugate in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a mono- or multi-specific molecule of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth or metastasis by at least about 20%, more preferably by at least about 40%, even more preferably by at least about 60%, and still more preferably by at least about 80% relative to untreated subjects. The ability of an agent or compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, metastasis, or otherwise ameliorate symptoms in a subject.

One of ordinary skill in the art would be able to determine such amounts based on such factors as the subject's size, the severity of the subject's symptoms, and the particular composition or route of administration selected.

XII. Administration

A composition of the invention can be administered via one or more routes of administration using one or more of a variety of methods known in the art. As will be appreciated by the skilled artisan, the route and/or mode of administration will vary depending upon the desired results. Preferred routes of administration for antibody drug conjugate of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a mono- or multi-specific molecule drug conjugate of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, and 4,596,556. Examples of well-known implants and modules useful in the present invention include those described in U.S. Pat. Nos. 4,487,603, 4,486,194, 4,447,233, 4,447,224, 4,439,196, and 4,475,196. These patents are incorporated herein by reference. Many other such implants, delivery systems, and modules are known to those skilled in the art.

XIII. Treatment Methods

In one aspect, the present invention relates to treatment of a subject in vivo using the above-described mono- or multi-specific molecule drug conjugate such that growth and/or metastasis of cancerous tumors is inhibited. In one embodiment, the invention provides a method of inhibiting growth and/or restricting metastatic spread of tumor cells in a subject, comprising administering to the subject a therapeutically effective amount of a mono- or multi-specific molecule drug conjugate.

Non-limiting examples of preferred cancers for treatment include chronic or acute leukemia including acute myeloid leukemia, chronic myeloid leukemia, acute lymphoblastic leukemia, chronic lymphocytic leukemia, lymphocytic lymphoma, breast cancer, ovarian cancer, melanoma (e.g., metastatic malignant melanoma), renal cancer (e.g. clear cell carcinoma), prostate cancer (e.g. hormone refractory prostate adenocarcinoma), colon cancer and lung cancer (e.g. non-small cell lung cancer). Additionally, the invention includes refractory or recurrent malignancies whose growth may be inhibited using the antibodies of the invention. Examples of other cancers that may be treated using the methods of the invention include bone cancer, pancreatic cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

As used herein, the term “subject” is intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of enhancement of an immune response. The methods are particularly suitable for treating human patients having a disorder that can be treated by augmenting the immune response.

The above treatment may also be combined with standard cancer treatments. For example, it may be effectively combined with chemotherapeutic regimes. In these instances, it may be possible to reduce the dose of chemotherapeutic reagent administered (Mokyr, M. et al. Cancer Res., 1998, 58, 5301-5304).

Other antibodies which may be used to activate host immune responsiveness can be used in or with the multi-specific molecule drug conjugate of this invention. These include molecules targeting on the surface of dendritic cells which activate DC function and antigen presentation. For example, anti-CD40 antibodies are able to substitute effectively for T cell helper activity (Ridge, J. et al. Nature, 1998, 393, 474-478) and can be used in conjunction with the multi-specific molecule drug conjugate of this invention (Ito, N. et al. Immunobiology, 2000, 201, 527-540). Similarly, antibodies targeting T cell costimulatory molecules such as CTLA-4 (U.S. Pat. No. 5,811,097), CD28 (Haan, J. et al. Immunol. Lett., 2014, 162, 103-112), OX-40 (Weinberg, A. et al. J. Immunol., 2000, 164, 2160-2169), 4-1BB (Melero, I. et al. Nature Med., 1997, 3, 682-685), and ICOS (Hutloff, A. et al. Nature, 1999, 397, 262-266) or antibodies targeting PD-1 (U.S. Pat. No. 8,008,449) and PD-L1 (U.S. Pat. Nos. 7,943,743; 8,168,179) may also provide for increased levels of T cell activation. In another example, the mono- or multi-specific molecule drug conjugate of this invention can be used in conjunction with anti-neoplastic antibodies, such as RITUXAN (rituximab), HERCEPTIN (trastuzumab), BEXXAR (tositumomab), ZEVALIN (ibritumomab), CAMPATH (alemtuzumab), LYMPHOCIDE (eprtuzumab), AVASTIN (bevacizumab), and TARCEVA (erlotinib), and the like.

### Definitions of Terms

The term “alkyl” as used herein refers to a hydrocarbon chain, typically ranging from about 1 to 25 atoms in length. Such hydrocarbon chains are preferably but not necessarily saturated and may be branched or straight chain, although typically straight chain is preferred.

The term C₁₋₁₀ alkyl includes alkyl groups with 1, 2, 3, 4, 5, 6, 7, 8, 9 and 10 carbons. Similarly C₁₋₂₅ alkyl includes all alkyls with 1 to 25 carbons. Exemplary alkyl groups include methyl, ethyl, isopropyl, n-butyl, n-pentyl, 2-methyl-1-butyl, 3-pentyl, 3-methyl-3-pentyl, and the like. As used herein, “alkyl” includes cycloalkyl when three or more carbon atoms are referenced. Unless otherwise noted, an alkyl can be substituted or unsubstituted.

The term “functional group” as used herein refers to a group that may be used, under normal conditions of organic synthesis, to form a covalent linkage between the entity to which it is attached and another entity, which typically bears a further functional group. A “bifunctional linker” refers to a linker with two functional groups forms two linkages via with other moieties of a conjugate.

The term “derivative” as used herein refers to a chemically-modified compound with an additional structural moiety for the purpose of introducing new functional group or tuning the properties of the original compound.

The term “protecting group” as used herein refers to a moiety that prevents or blocks reaction of a particular chemically reactive functional group in a molecule under certain reaction conditions. Various protecting groups are well-known in the art and are described, for example, in T. W. Greene and G. M. Wuts, Protecting Groups in Organic Synthesis, Third Edition, Wiley, New York, 1999, and in P. J. Kocienski, Protecting Groups, Third Ed., Thieme Chemistry, 2003, and references cited therein.

The term “PEG” as used herein refers to polyethylene glycol. PEGs for use in the present invention typically comprise a structure of —(CH₂CH₂O)ₙ—. PEGs may have a variety of molecular weights, structures or geometries. A PEG group may comprise a capping group that does not readily undergo chemical transformation under typical synthetic reaction conditions. Examples of capping groups include —OC₁₋₂₅ alkyl or —OAryl.

The term “PEGylate” as used herein refers to chemical modification by polyethylene glycol.

The term “linker” as used herein refers to an atom or a collection of atoms used to link interconnecting moieties, such as an antibody and a polymer moiety. A linker can be cleavable or noncleavable. The preparation of various linkers for conjugates have been described in literatures including for example Goldmacher et al., Antibody-drug Conjugates and Immunotoxins: From Pre-clinical Development to Therapeutic Applications, Chapter 7, in Linker Technology and Impact of Linker Design on ADC properties, Edited by Phillips G L; Ed. Springer Science and Business Media, New York (2013). Cleavable linkers incorporate groups or moieties that can be cleaved under certain biological or chemical conditions. Examples include enzymatically cleavable disulfide linkers, 1,4- or 1,6-benzyl elimination, trimethyl lock system, bicine-based self cleavable system, acid-labile silyl ether linkers and other photo-labile linkers.

The term “linking group” or “linkage group” as used herein refers to a functional group or moiety connecting different moieties of a compound or conjugate. Examples of a linking group include, but are not limited to, amide, ester, carbamate, ether, thioether, disulfide, hydrazone, oxime, and semicarbazide, carbodiimide, acid labile group, photolabile group, peptidase labile group and esterase labile group. For example, a linker moiety and a polymer moiety may be connected to each other via an amide or carbamate linkage group.

The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

An “isolated” peptide, polypeptide, or protein refers to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis.

An isolated polypeptide/protein described in the invention can be purified from a natural source, produced by recombinant DNA techniques, or by chemical methods.

An “antigen” refers to a substance that elicits an immunological reaction or binds to the products of that reaction. The term “epitope” refers to the region of an antigen to which an antibody or T cell binds.

The term “antibody” as referred to herein includes whole antibodies and any antigen binding fragment or single chains thereof. Whole antibodies are glycoproteins comprising at least two heavy (H) chains and two light (L) chains inter-connected by disulfide bonds. Each heavy chain is comprised of a heavy chain variable region (V_{H}) and a heavy chain constant region. The heavy chain constant region is comprised of three domains, C_{H}1, C_{H}2 and C_{H}3. Each light chain is comprised of a light chain variable region (V_{L}) and a light chain constant region (C_{L}), the light chain constant region is comprised of one domain. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

As used herein, “antibody fragments”, may comprise a portion of an intact antibody, generally including the antigen binding and/or variable region of the intact antibody and/or the Fc region of an antibody which retains FcR binding capability. Examples of antibody fragments include linear antibodies; single-chain antibody molecules; and multispecific antibodies formed from antibody fragments.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L} and C_{H}I domains; (ii) a F(ab′)₂ fragment, a bivalent fragment comprising two Fab fragments linked by a disulfide bridge at the hinge region; (iii) a Fab′ fragment, which is essentially an Fab with part of the hinge region; (iv) a Fd fragment consisting of the V_{H} and C_{H}I domains; (v) a Fv fragment consisting of the V_{L} and V_{H} domains of a single arm of an antibody, (vi) a dAb, which consists of a V_{H} domain; (vii) an isolated complementarity determining region (CDR); and (viii) a nanobody, a heavy chain variable region containing a single variable domain and two constant domains.

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. Science 1988, 242, 423-426; and Huston et al. Proc. Natl. Acad. Sci. USA 1988, 85, 5879-5883. Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

As used herein, the term “Fc fragment” or “Fc region” is used to define a C-terminal region of an immunoglobulin heavy chain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein (Kohler, G. et al. Nature, 1975, 256, 495-497), which is incorporated herein by reference, or may be made by recombinant DNA methods (U.S. Pat. No. 4,816,567), which is incorporated herein by reference. The monoclonal antibodies may also be isolated from phage antibody libraries using the techniques described by Clackson et al., Nature, 1991, 352, 624-628 and Marks et al., J Mol Biol, 1991, 222, 581-597, for example, each of which is incorporated herein by reference.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; Morrison et al., Proc Natl Acad Sci USA, 1984, 81, 6851-6855; Neuberger et al., Nature, 312, 1984, 604-608; Takeda et al., Nature, 1985, 314, 452-454; International Patent Application No. PCT/GB85/00392, each of which is incorporated herein by reference).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody optionally also will comprise at least a portion of an immunoglobulin constant region (Fc), typically that of a human immunoglobulin. For further details, see Jones et al., Nature, 1986, 321, 522-525; Riechmann et al., Nature, 1988, 332, 323-329; Presta, Curr Op Struct Biol, 1992, 2, 593-596; U.S. Pat. No. 5,225,539, each of which is incorporated herein by reference.

“Human antibodies” refer to any antibody with fully human sequences, such as might be obtained from a human hybridoma, human phage display library or transgenic mouse expressing human antibody sequences.

The term “pharmaceutical composition” refers to the combination of an active agent with a carrier, inert or active, making the composition especially suitable for diagnostic or therapeutic use in vivo or ex vivo.

As used herein, “pharmaceutically acceptable carrier” includes any and all solvents, dispersion media, coatings, antibacterial and antifungal agents, isotonic and absorption delaying agents, and the like that are physiologically compatible. A “pharmaceutically acceptable carrier”, after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see e.g., Berge, S. M., et al. J. Pharm. Sci. 1997, 66,1-19).

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

An “effective amount” refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. A therapeutically effective amount of a combination to treat a neoplastic condition is an amount that will cause, for example, a reduction in tumor size, a reduction in the number of tumor foci, or slow the growth of a tumor, as compared to untreated animals.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

### EXAMPLES

The following examples serve to provide further appreciation of the invention but are not meant by any way to restrict the effective scope of the invention.

### Example 1. Preparation of 30 kmPEG-Lys(Mal)-(Val-Cit-PAB-MMAE)₄

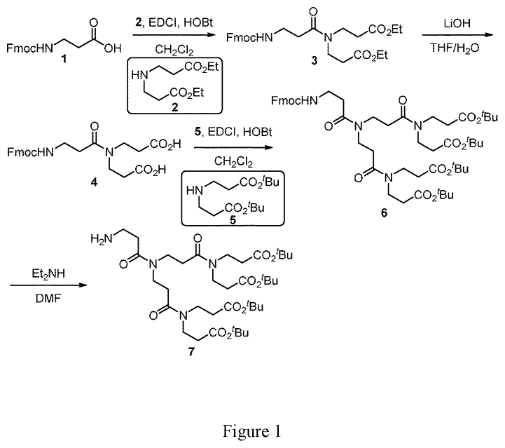

### Preparation of Branched Linker Intermediate Compound 7 (FIG. 1)

To a solution of 1 (3.1 g, 10 mmol) in dry CH₂Cl₂ (50 mL) at RT under Argon, 2 (2.6 g, 12 mmol, 1.2 eq), EDCI (2.87 g, 15 mmol, 1.5 eq) and HOBt (0.27 g, 2 mmol, 0.2 eq) are added.

The mixture is stirred until full conversion is observed by TLC. After reaction is completed, the mixture is extracted with CH₂Cl₂, and the organic layer is washed with brine, dried over Na₂SO₄, filtered and concentrated in vacuo. The crude reaction mixture is purified through chromatography on silica gel to yield the product 3.

To a solution of 3 (2.6 g, 5 mmol) in THF (50 mL) at RT under Argon, 1M LiGH (20 mL, 20 mmol, 4.0 eq) is added. The mixture is stirred until full conversion is observed by TLC. After reaction is completed, the mixture is extracted with CH₂Cl₂, and the organic layer is washed with brine, dried over Na₂SO₄, filtered and concentrated in vacuo. The crude reaction mixture is purified through chromatography on silica gel to yield the product 4.

To a solution of 4 (2.3 g, 5 mmol) in dry CH₂Cl₂ (50 mL) at RT under Argon, 5 (1.6 g, 6 mmol, 1.2 eq), EDCI (1.4 g, 7.5 mmol, 1.5 eq) and HOBt (0.14 g, 1 mmol, 0.2 eq) are added. The mixture is stirred until full conversion is observed by TLC. After reaction is completed, the mixture is extracted with CH₂Cl₂, and the organic layer is washed with brine, dried over Na₂SO₄, filtered and concentrated in vacuo. The crude reaction mixture is purified through chromatography on silica gel to yield the product 6.

Diethylamine (1.0 ml) is added to a solution of 6 (0.97 g, 1.0 mmol) in DMF (10 ml) and the reaction is allowed to proceed at RT for 1.5 h. The diethylamine and the solvent are removed in vacuo at a bath temperature not exceeding 30° C. The residue is triturated with ether (25 ml). The solids precipitated are collected, filtered and washed with ether twice, (2×20 ml) and dried in vacuo to give the product 7.

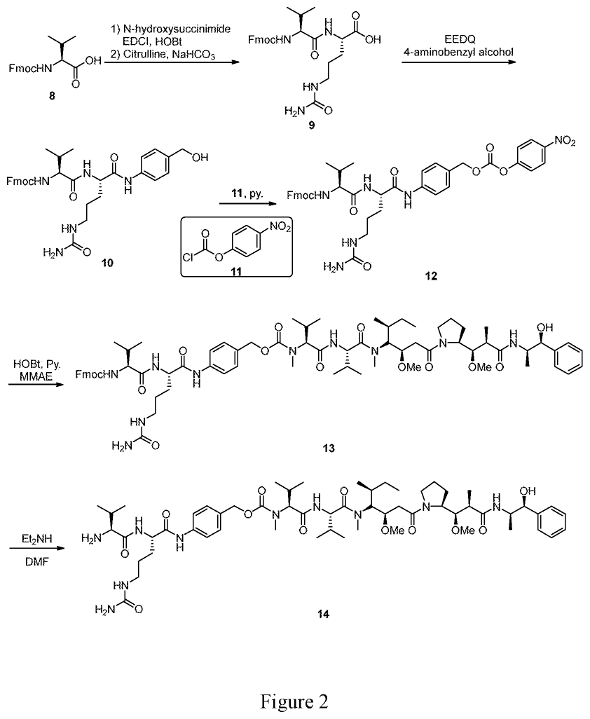

### Preparation of Compound 14 Val-Cit-PAB-MMAE (FIG. 2)

Fmoc-Val-OH 8 (3.4 g, 10 mmol, 1.0 eq), N-hydroxysuccinimide (1.5 g, 13 mmol, 1.3 eq) are dissolved in a mixture of CH₂Cl₂ (60 ml) and THF (20 ml) at 0° C., and EDCI (2.5 g, 13 mmol, 1.3 eq) is added to the above solution. The solution is then slowly warmed to RT. The reaction mixture is stirred at RT until reaction is complete. The reaction mixture is then concentrated under reduced pressure. The concentrated residue is dissolved with THF and filtered to remove EDU. The filtrate is concentrated and re-slurried with n-heptane at 5-10° C. for 12 hours. Solids are filtered, washed and dried under vacuum to give the Fmoc-Val-OSu.

Fmoc-Val-OSu (4.4 g, 10 mmol, 1.0 eq) is dissolved in acetonitrile (50 mL) at RT followed by the addition of the solution of sodium carbonate (1.2 g, 11 mmol, 1.1 eq) and L-citrulline (1.9 g, 11 mmol, 1.1 eq) in water (50 ml). The reaction mixture is stirred at 35° C. for several hours until reaction is complete. The mixture is cooled to 20° C., quenched with 15% citric acid (150 mL), and extracted with EtOAc/ⁱ-PrOH (9:1) (200 mL×2). The combined organic phase is washed with water (140 mL), dried with anhydrous Na₂SO₄, and concentrated. The residue is washed with methyl tert-butyl ether to yield the Fmoc-Val-Cit-OH 9.

Fmoc-Val-Cit-OH 9 (3.0 g, 6.0 mmol, 1.0 eq) and 4-aminobenzyl alcohol (1.5 g, 12.1 mmol, 2.0 eq) are dissolved in the solution of CH₂Cl₂ (70 mL) and MeOH (30 mL). EEDQ (3.0 g, 12.1 mmol, 2.0 eq) is added and the solution is stirred at RT for 1 day. Additional EEDQ (1.5 g, 6.0 mmol, 1.0 eq) is added and the solution is continuously stirred for 12 hours. The reaction mixture is concentrated and the residue is washed with methyl tert-butyl ether to give the Fmoc-Val-Cit-PAB-OH 10.

To a solution of Fmoc-Val-Cit-PAB-OH 10 (2.0 g, 3.3 mmol, 1.0 eq) in DMF (20 mL), p-nitrobenzoyl chloride 11 (1.2 g, 6.6 mmol, 2.0 eq) and pyridine (0.4 mL, 5.0 mmol, 1.5 eq) are added. The mixture is stirred at RT for 12 hours and then concentrated. The residue is washed with EtOAc/methyl tert-butyl ether to give the product 12.

To a solution of 12 (1.3 g, 1.7 mmol, 1.0 eq) in DMF (3.4 mL), HOBt (376 mg, 2.78 mml, 1.6 eq) and pyridine (0.85 mL) are added at RT. followed by MMAE (1.0 g, 1.39 mmol). The solution is stirred at RT for 24 hours. The reaction mixture is concentrated and the residue is purified through chromatography on silica gel to give the product Fmoc-Val-Cit-PAB-MMAE 13.

To a solution of Fmoc-Val-Cit-PAB-MMAE 13 (1.4 g, 1.1 mmol) in DMF (20 mL), Et₂NH (5 mL) is added, and the solution is stirred at RT for 12 hours. The reaction mixture is concentrated and the residue is washed with EtOAc/methyl tert-butyl ether to give the product 14.

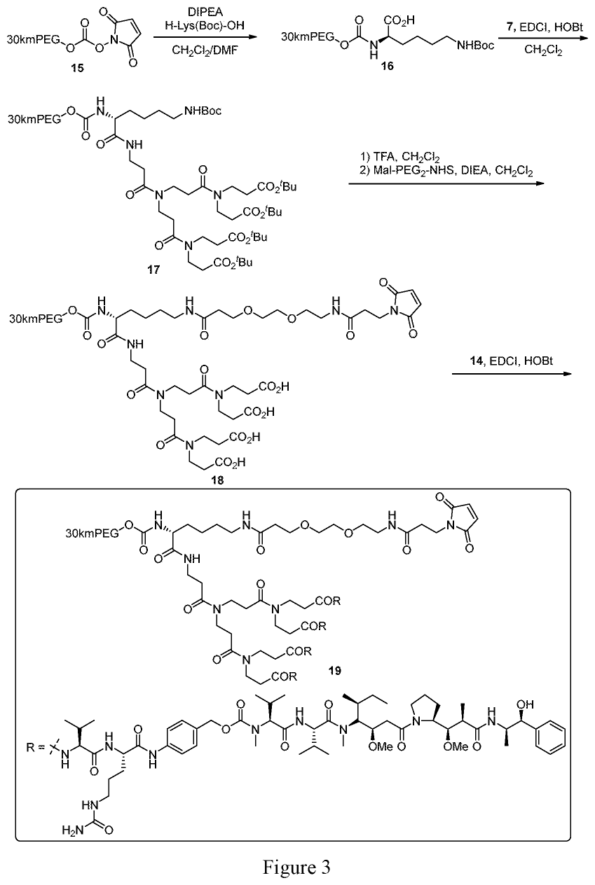

Preparation of Compound 19 30 k mPEG-Lys(Mal)-(MMAE)₄ (FIG. 3)

H-Lys(boc)-OH (369 mg, 1.5 mmol, 3.0 eq) is added into 100 mL anhydrous DMF followed by DIEA (5.0 mmol, 10.0 eq), compound 15 (15 g, 0.5 mmol, 1.0 eq) and 150 mL anhydrous CH₂Cl₂. The mixture is stirred under Argon at RT overnight. The insoluble materials are filtered off. The solvent is removed and the residue is recrystallized from CH₂Cl₂/methyl tert-butyl ether. The isolated solids are recrystallized again from ACN/2-propanol. The product is dried at 40° C. over 4 h under vacuum to give the product 16.

To a solution of 16 (15 g, 0.5 mmol) in dry CH₂Cl₂ (150 mL) at RT under Argon, 7 (1.1 g, 1.5 mmol, 3.0 eq), EDCI (0.58 g, 3.0 mmol, 6.0 eq) and HOBt (0.61 g, 4.5 mmol, 9.0 eq) are added. The mixture is stirred under Argon at RT overnight. The solvent is removed and the residue is recrystallized from CH₂Cl₂/methyl tert-butyl ether. The solids precipitated are recrystallized again from ACN/2-propanol. The product is dried at 40° C. over 4 h under vacuum to give the product 17.

17 (9.0 g, 0.3 mmol) is dissolved in CH₂Cl₂ (90 mL) followed by addition of TFA (45 mL). The mixture is stirred at RT for 1 hr. The solvent is removed under vacuum as much as possible at <35° C. The residue is recrystallized from CH₂Cl₂/ methyl tert-butyl ether twice. The product is dried under vacuum at 40° C. to produce an intermediate. The dried intermediate (6.0 g, 0.2 mmol, 1.0 eq) is then dissolved in anhydrous CH₂Cl₂(60 mL) under Argon. The solution is cooled to 0-5° C., DIPEA (517 mg, 4 mmol, 20 eq) and NHS-PEG₂-Mal (0.22 g, 0.5 mmol, 2.5 eq) are added at 0-−5° C. The mixture is stirred at 0-5° C. for 2 h., then allowed to warm up slowly to RT and kept at RT under Argon overnight. After reaction, the solvent is removed and the residue is recrystallized from CH₂Cl₂/ methyl tert-butyl ether. The solids precipitated are recrystallized again from ACN/2-propanol. The isolated product is dried at 40° C. over 4 h under vacuum to give the product 18.

To a solution of 18 (3.0 g, 0.1 mmol) in dry CH₂Cl₂ (30 mL) at RT under Argon, 14 (0.9 g, 0.8 mmol, 8.0 eq), EDCI (0.46 g, 2.4 mmol, 24 eq) and HOBt (0.49 g, 3.6 mmol, 36 eq) are added. The mixture is stirred under Argon at RT overnight. The solvent is removed and the residue is recrystallized from CH₂Cl₂/methyl tert-butyl ether. The solids precipitated are recrystallized again from ACN/2-propanol. The isolated product is dried at 40° C. over 4 h under vacuum to give the product 19.

### Example 2 Preparation of SCAHer2×SCAHer2

Bispecific single chain antibody (SCA) fragments of anti-Her2 (SCAHer2)-1 and anti-Her2 (SCAHer2)-2 can be prepared via recombinant DNA technology in mammalian cells (e.g., CHO using EasySelect™) or yeast (e.g., Pichia pastori Expression Kit containing a pPICZ vector). DNA Sequences of SCAHer2-1×SCAH2-2 corresponding to amino acid sequence below (SEQ ID NO: 1) are synthesized and cloned into the expression vectors and transformed in the host cells. Expressed protein is purified by Ni-chelating resin or protein L resin. To facilitate the subsequent conjugation, a site specific conjugation functional group thiol is inserted through recombinant DNA technology into the linker between two Her2 SCAs.

Amino acid Sequence of SCAHer2IIxSCAHer2IV
(SEQ ID NO: 1):
DIQMTQSPSSLSASVGDRVTITCKASQDVSIGVAWYQQKPGKAPKLLIY
SASYRYTGVPSRFSGSGSGTDFTLTISSLQPEDFATYYCQQYYIYPYTF
GQGTKVEIKRTGGSGGSGGSGGSGGEVQLVESGGGLVQPGGSLRLSCAA
SGFTFTDYTMDWVRQAPGKGLEWVADVNPNSGGSIYNQRFKGRFTLSVD
RSKNTLYLQMNSLRAEDTAVYYCARNLGPSFYFDYWGQGTLVTVSSGCG
SGGSGGSGGSGGEVQLVESGGGLVQPGGSLRLSCAASGFNIKDTYIHWV
RQAPGKGLEWVARIYPTNGYTRYADSVKGRFTISADTSKNTAYLQMNSL
RAEDTAVYYCSRWGGDGFYAMDYWGQGTLVTVSSGGSGGSGGSGGSGGD
IQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFG
QGTKVEIKRTHHHHHH

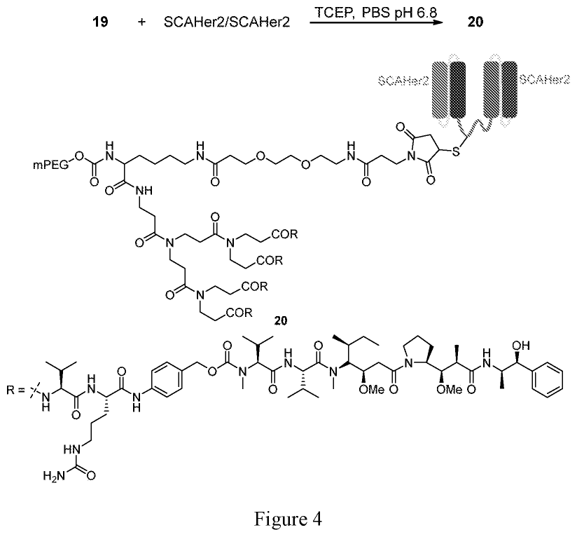

### Example 3 Preparation of 30 kmPEG-(SCAHer2×SCAHer2)-(Val-Cit-PAB-MMAE)₄ (FIG. 4)

Protein SCAHer2/SCAHer2 is treated by reducing agent TCEP-HCl in PBS buffer (pH=7.4) at room temperature for 30 min before pH adjustment to pH 6.8 with a pH=4.12 stock solution of 500 mM sodium phosphate. The treated protein is concentrated to 5 mg/mL before pegylation. Pegylation of SCAHer2/SCAHer2 is conducted at room temperature for 3 hours with 5 to 10 mole equivalent of compound 19 [30 k mPEG-Lys(Mal)-(Val-Cit-PAB-MMAE)₄]. The reaction is quenched with 10 mM of L-cystine at room temperature for 10 min. Final product PEG-Lys(SCAHer2/SCAHer2)-(Val-Cit-PAB-MMAE)₄ is purified with cation exchange chromatography column (CM Fast Flow) at pH 6.5 in 20 mM phosphate buffer. The target compound 20 is confirmed by SEC-HPLC and cell-based activity assay.

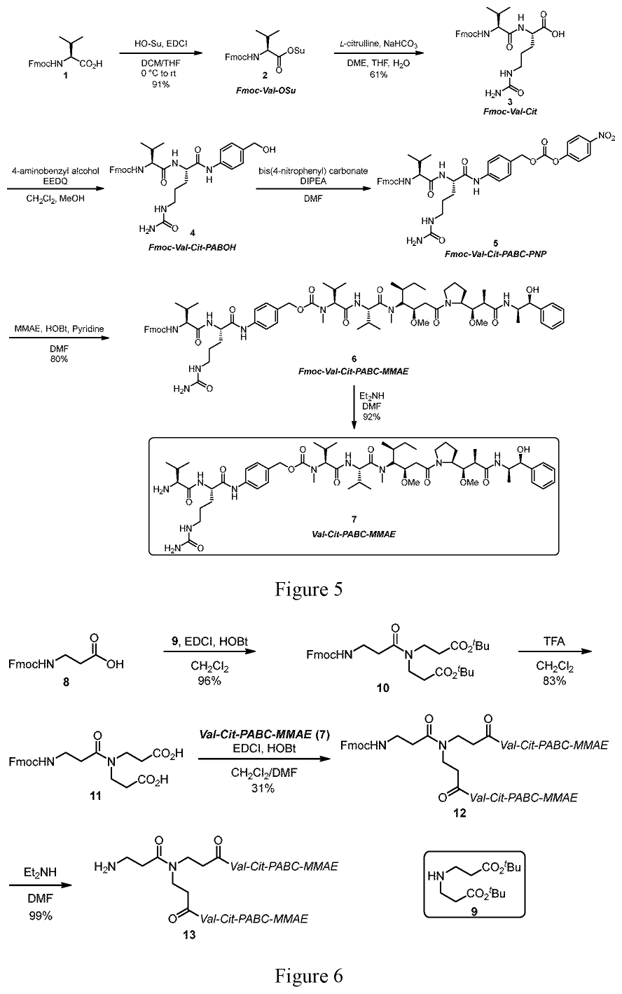

### Example 4. Preparation of Val-Cit-PABC-MMAE (FIG. 5)

Fmoc-Val-OSu (compound 2): Fmoc-Val-OH (20.3 g, 60.0 mmol) and N-hydroxysuccinimide (9.0 g, 78.0 mmol) were dissolved in a mixture of CH₂Cl₂ (120 mL) and THF (40 mL). Separately, EDCI (13.8 g, 72.0 mmol) was dissolved in CH₂Cl₂ (200 mL) and the solution was cooled to 0-5° C. The Fmoc-Val-OH/NHS solution was then added to the EDCI solution followed by warming up the reaction mixture to room temperature. The reaction mixture was stirred at room temperature until reaction was completed. The reaction mixture was then concentrated under reduced pressure as much as possible and the residue CH₂Cl₂ was chased out with THF (2×100 mL). The concentrated residue was dissolved with THF (800 mL) and filtered to remove EDU.

The filtrate was concentrated under reduced pressure and the residue was slurried with n-heptane (800 mL) at 5-10° C. for 12 h. Solids were filtered, washed and dried under vacuum to yield Fmoc-Val-OSu (2) (23.8 g, 91%) as white powder. HRMS (ESI) caled. For C₂₄H₂₄N₂O₆Na [M+Na]⁺ 459.1532, found 459.1523.

Fmoc-Val-Cit (compound 3): Fmoc-Val-Osu (9.8 g, 22.5 mmol) was dissolved in DME (150 mL) at room temperature. Separately, sodium bicarbonate (2.1 g, 24.7 mmol) was dissolved in water (150 mL) at room temperature followed by the addition of L-citrulline (4.3 g, 24.7 mmol) to give a homogeneous clear solution. The prepared L-citrulline solution was then added to the Fmoc-Val-Osu solution. THF (75 mL) was added and the reaction mixture was stirred at room temperature for 16 h until reaction was completed. The reaction mixture was acidified with 15% citric acid (200 mL) and concentrated with Rotavapor. The residue was suspended in water (500 mL) for 2 h before filtered and dried in vacuum. Dried solids were re-suspended in methyl tert-butyl ether (500 mL) and stirred for 12 h before being filtered, washed and dried under vacuum to yield Fmoc-Val-Cit (3) (6.8 g, 61%) as white powder. HRMS (ESI) calcd. For C₂₆H₃₃N₄O₆ [M+H]⁺ 497.2400, found 497.2388.

Fmoc-Val-Cit-PABOH (compound 4): A solution of compound 3 (4.96 g, 10.0 mmol) and 4-aminobenzyl alcohol (2.46 g, 20.0 mmol) in CH₂Cl₂ (350 mL) and MeOH (150 mL) was added by EEDQ (4.95 g, 20.0 mmol). The reaction mixture was stirred at room temperature for 24 h. Additional EEDQ (2.5 g, 10.0 mmol) was added to the reaction and mixture was stirred for another 24 h. After the reaction was completed, the solvent was removed under reduced pressure and the resulting residue was slurried in methyl tert-butyl ether (800 mL) for 12 h. Solids were filtered, washed and dried under vacuum to yield compound 4 (4.1 g, 69%) as white powder. HRMS (ESI) calcd. For C₃₃H₄₀N₅O₆ [M+H]⁺ 602.2979, found 602.2969.

Fmoc-Val-Cit-PABC-PNP (compound 5): To a solution of compound 4 (5.2 g, 8.6 mmol) and bis(4-nitrophenyl) carbonate (4.9 g, 16.1 mmol) in DMF (52 mL) at room temperature was added DIPEA (2.5 mL, 15.0 mmol). The reaction mixture was stirred at room temperature for 5 h until reaction was completed. The product was precipitated by addition of anhydrous ethyl acetate (250 mL) and methyl tert-butyl ether (250 mL). The suspension was cooled to 0° C. and stirred for 30 min. The solids were isolated by filtration, washed and dried under vacuum to yield Fmoc-Val-Cit-PABC-PNP (5) (4.7 g, 72%) as pale yellow powder. HRMS (ESI) calcd. For C₄₀H₄₃N₆O₁₀ [M+H]⁺ 767.3041, found 767.3045.

Fmoc-Val-Cit-PABC-MMAE (compound 6): Compound MMAE (2.0 g, 1.8 mmol) and Fmoc-Val-Cit-PABC-PNP (5) (2.8 g, 3.6 mmol) were dissolved in DMF (20 m L). HOBt (0.75 g, 5.6 mmol) and pyridine (1.7 mL) were then added and the reaction mixture was stirred at room temperature for 24 h. After the reaction was completed, the reaction mixture was cooled to 0° C. followed by the addition of methyl tert-butyl ether (180 mL) to precipitate product. The slurry was stirred for 3-5 h and filtered, washed and dried under vacuum. The crude product was purified by column purification to yield Fmoc-Val-Cit-PABC-MMAE (6) (3.0 g, 80%) as yellow powder. HRMS (ESI) calcd. For C₇₃H₁₀₅N₁₀O₁₄ [M+H]⁺ 1345.7812, found 1345.7820.

Val-Cit-PABC-MMAE (compound 7): Compound 6 (3.0 g, 2.2 mmol) was suspended in anhydrous DMF (40 mL) and stirred at room temperature until a homogeneous suspension formed. Diethylamine (10 mL) was then added and the reaction mixture was stirred at room temperature for 3 h. After reaction was completed, methyl tert-butyl ether (100 mL) and ethyl acetate (50 mL) were added over 60 min. The resulting mixture was stirred for 4 h at 0° C. Solids were filtered and dried under vacuum to yield Val-Cit-PABC-MMAE (7) (2.3 g, 92%) as pale yellow powder. HRMS (ESI) calcd. For C₅₈H₉₅N₁₀O₁₂ [M+H]⁺ 1123.7131, found 1123.7142.

### Example 5. Preparation of Compound 13 (Branch Linker B with 2×MMAE) (FIG. 6)

Compound 10: To a solution of compound 8 (0.62 g, 2.0 mmol) in dry CH₂Cl₂ (15 mL) at room temperature under argon, Di-tert-butyl 3,3′-azanediyldipropanoate (9) (0.62 mL, 2.2 mmol), EDCI (0.58 g, 3.0 mmol) and HOBt (54 mg, 0.4 mmol) were added. The mixture was stirred at room temperature and monitored by TLC. After the reaction was completed, the mixture was extracted with CH₂Cl₂ (30 mL×2), and the organic layers were combined, washed with brine (20 mL) and dried over Na₂SO₄. The solution was concentrated with Rotavapor. The crude reaction mixture was purified through chromatography on silica gel to yield the compound 10 (1.1 g, 96%) as colorless oil. HRMS (ESI) calcd. For C₃₂H₄₃N₂O₇ [M+H]⁺ 567.3070, found 567.3062.

Compound 11: Compound 10 (5.2 g, 9.2 mmol) was dissolved in CH₂Cl₂ (100 mL) followed by addition of TFA (25 mL). The mixture was stirred at room temperature for 3 h. The solvent was removed under vacuum as much as possible at <35° C. The residue was purified through chromatography on silica gel to yield the compound 11 (3.4 g, 83%) as colorless oil. HRMS (ESI) calcd. For C₂₄H₂₇N₂O₇ [M+H]⁺ 455.1818, found 455.1824.

Compound 12: To a stirred solution of compound 11 (41 mg, 0.091 mmol) in dry CH₂Cl₂ (2 mL) and DMF (2 mL) at room temperature under argon, Val-Cit-PABC-MMAE (7) (224 mg, 0.2 mmol), EDCI (52 mg, 0.27 mmol) and HOBt (5 mg, 0.04 mmol) were added. The mixture was stirred at room temperature and monitored by TLC. After the reaction was completed, the mixture was concentrated in vacuum. The residue was purified by preparative HPLC with Welch Ultimate XB-C18 column (eluents: A=0.1% TFA in water, B=MeCN) to yield the compound 12 (74 mg, 31%) as pale yellow solid. HRMS (ESI) calcd. For C₁₄₀H₂₁₂N₂₂O₂₉ [M+2H]2+1333.2912, found 1333.2907.

Compound 13: Diethylamine (0.6 ml) was added to a solution of compound 12 (73 mg) in DMF (3 ml). The reaction was allowed to proceed at room temperature for 4 h. The reaction mixture was concentrated with Rotavapor and the residue was purified by preparative HPLC using Welch Ultimate XB-C18 column (eluents: A=0.1% TFA in water, B=MeCN) to yield the compound 13 (71 mg, 99%) as pale yellow solid. HRMS (ESI) calcd. For C₁₂₅H₂₀₂N₂₂O₂₇ [M+2H]2+1222.2572, found 1222.2560.

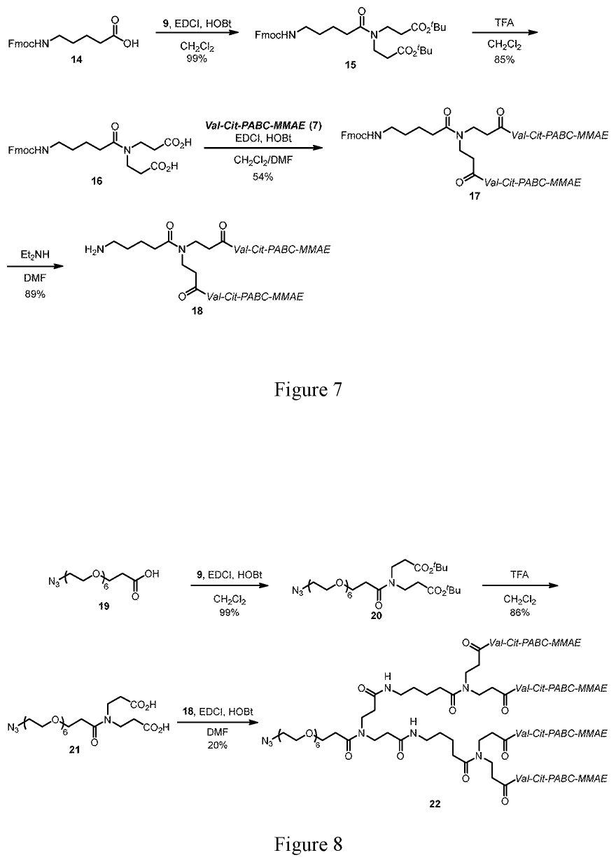

### Example 6. Preparation of Compound 18 (Branch Linker B with 2×MMAE) (FIG. 7)

Compound 15: To a solution of compound 14 (0.68 g, 2.0 mmol) in dry CH₂Cl₂ (10 mL) at room temperature under argon, Di-tert-butyl 3,3′-azanediyldipropanoate (9) (0.64 mL, 2.2 mmol), EDCI (0.58 g, 3.0 mmol) and HOBt (54 mg, 0.4 mmol) were added. The mixture was stirred at room temperature and monitored by TLC. After the reaction was completed, the mixture was extracted with CH₂Cl₂ (2×30 mL), and the combined organic layer was washed with brine (20 mL), dried over Na₂SO₄, filtered and concentrated with Rotavapor. The residue was purified by chromatography on silica gel to yield the compound 15 (1.2 g, 99%) as colorless oil. IRMS (ESI) calcd. for C₃₄H₄₇N₂O₇ [M+H]⁺ 595.3383, found 595.3380.

Compound 16: Compound 15 (0.5 g, 0.84 mmol) was dissolved in CH₂Cl₂ (6.0 mL) followed by addition of TFA (3.0 mL). The mixture was stirred at room temperature for 3 h. The solvent was removed under vacuum as much as possible at <35° C. The residue was purified by chromatography on silica gel to yield the compound 16 (0.34 g, 85%) as colorless oil. HRMS (ESI) calcd. for C₂₆H₃₁N₂O₇ [M+H]⁺ 483.2131, found 483.2127.

Compound 17: To a solution of compound 16 (185 mg, 0.383 mmol) in a mixture of dry CH₂C12 (8 mL) and DMF (8 mL) at room temperature under argon, Val-Cit-PABC-MMAE (7) (947 mg, 0.843 mmol), EDCI (238 mg, 1.23 mmol) and HOBt (26 mg, 0.19 mmol) were added. The mixture was stirred at room temperature and monitored by HPLC. After the reaction was completed, the mixture was concentrated with Rotavapor. The residue was purified by preparative HPLC using Welch Ultimate XB-C18 column (eluents: A=0.1% TFA in water, B=MeCN) to yield the compound 17 (0.56 g, 54%) as pale yellow solid. IRMS (ESI) calcd. for C₁₄₂H₂₁₆N₂₂O₂₉ [M+H]⁺ 2694.6137, found 2694.6146.

Compound 18: Diethylamine (2.0 ml) was added to a solution of compound 17 (0.62 g) in DMF (5 ml). The reaction mixture was allowed to proceed at room temperature for 2 h. The reaction mixture was concentrated with Rotavapor and the residue was purified by preparative HPLC using Welch Ultimate XB-C18 column (eluents: A=0.1% TFA in water, B=MeCN) to yield the compound 18 (0.51 g, 89%) as pale yellow solid. HRMS (ESI) calcd. for C₁₂₇H₂₀₅N₂₂O₂₇ [M+H]+ 2471.5378, found 2471.5369; calcd. for C₁₂₇H₂₀₆N₂₂O₂₇ [M+2H]2+1236.2728, found 1236.2744.

### Example 7. Preparation of Compound 22 (Branch Linker B with 4×MMAE) (FIG. 8)

Compound 20: To a solution of compound 19 (0.76 g, 2.0 mmol) in dry CH₂Cl₂ (10 mL) at room temperature under argon, Di-tert-butyl 3,3′-azanediyldipropanoate (9) (0.64 mL, 2.2 mmol), EDCI (0.58 g, 3.0 mmol) and HOBt (54 mg, 0.4 mmol) were added. The mixture was stirred at room temperature and monitored by TLC. After the reaction was completed, the mixture was extracted with CH₂Cl₂ (2×30 mL), and the combined organic layer was washed with brine (20 mL), dried over Na₂SO₄, filtered and concentrated with Rotavapor. The crude reaction mixture was purified by chromatography on silica gel to yield the compound 20 (1.2 g, 99%) as colorless oil. IRMS (ESI) calcd. for C₂₉H₅₅N₄O₁₁ [M+H]⁺ 635.3867, found 635.3860.

Compound 21: Compound 20 (0.3 g, 0.47 mmol) was dissolved in CH₂Cl₂ (4.0 mL) followed by addition of TFA (2.0 mL). The mixture was stirred at room temperature for 3 h. The solvent was removed under vacuum as much as possible at <35° C. The residue was purified by chromatography on silica gel to yield the compound 21 (0.34 g, 85%) as colorless oil. HIRMS (ESI) calcd. for C₂₁H₃₉N₄O₁₁ [M+H]⁺ 523.2615, found 523.2607.

Compound 22: To a stirred solution of compound 21 (39 mg, 0.076 mmol) in a mixture of dry CH₂Cl₂ (2 mL) and DMF (2 mL) at room temperature under argon, compound 18 (0.41 g, 0.17 mmol), EDCI (43 mg, 0.23 mmol) and HOBt (4.0 mg, 0.03 mmol) were added. The reaction mixture was stirred at room temperature and monitored by HPLC. After the reaction was completed, the mixture was concentrated with Rotavapor. The residue was purified by preparative HPLC with Welch Ultimate XB-C18 column (eluents: A=0.1% TFA in water, B=MeCN) to yield the compound 22 (81 mg, 20%) as pale yellow solid. HRMS (ESI) calcd. for C₂₇₅H₄₄₅N₄₈O₆₃ [M+3H]³+1810.1053, found 1810.1061; calcd. for C₂₇₅H₄₄₆N₄₈O₆₃ [M+4H]⁴+1357.8310, found 1357.8346.

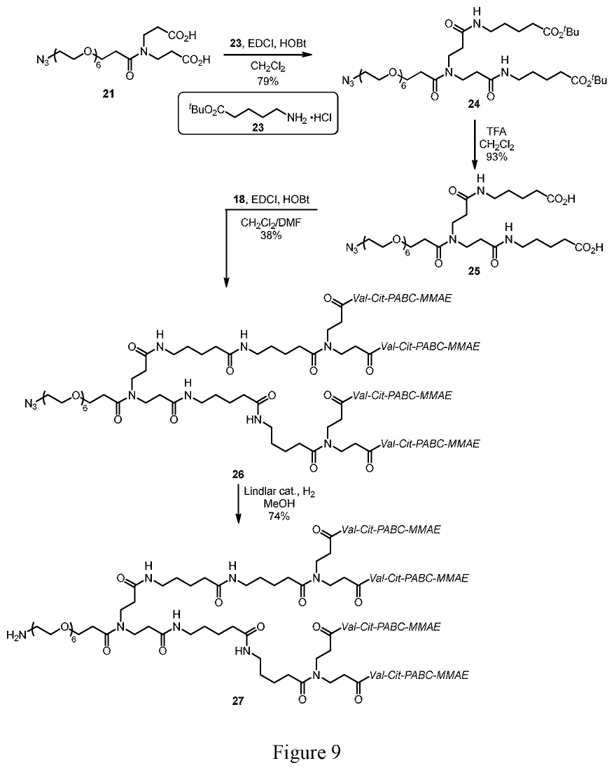

### Example 8. Preparation of Compound 27 (branch linker B with 4×MMAE) (FIG. 9)

Compound 24: To a solution of compound 21 (0.57 g, 1.1 mmol) in dry CH₂Cl₂ (10 mL) at room temperature under argon, compound 23 (0.51 g, 2.4 mmol), EDCI (0.67 g, 3.5 mmol), HOBt (74 mg, 0.55 mmol) and DIPEA (0.78 mL, 4.4 mmol) were added. The mixture was stirred at room temperature and monitored by TLC. After the reaction was completed, the mixture was extracted with CH₂Cl₂ (2×30 mL), and the combined organic layer was washed with brine (20 mL), dried over Na₂SO₄, filtered and concentrated with Rotavapor. The residue was purified by chromatography on silica gel to yield the compound 24 (0.7 g, 79%) as colorless oil. HRMS (ESI) calcd. for C₃₉H₇₃N₆O₁₃ [M+H]⁺ 833.5236, found 833.5231.

Compound 25: Compound 24 (0.52 g, 0.62 mmol) was dissolved in CH₂Cl₂ (5.0 mL) followed by addition of TFA (2.0 mL). The mixture was stirred at room temperature for 3 h. The solvent was removed under vacuum as much as possible at <35° C. The residue was purified by chromatography on silica gel to yield the compound 25 (0.42 g, 93%) as colorless oil. HRMS (ESI) calcd. for C₃₁H₅₇N₆O₁₃ [M+H]⁺ 721.3984, found 721.3997.

Compound 26: To a solution of compound 25 (77 mg, 0.11 mmol) in DMF (5 mL) at room temperature under argon, compound 18 (0.58 g, 0.24 mmol), EDCI (82 mg, 0.43 mmol) and HOBt (14 mg, 0.11 mmol) were added. The mixture was stirred at room temperature and monitored by HPLC. After the reaction was completed, the mixture was concentrated with Rotavapor. The crude reaction mixture was purified by preparative HPLC using Welch Ultimate XB-C18 column (eluents: A=0.1% TFA in water, B=MeCN) to yield the compound 26 (0.23 g, 38%) as pale yellow solid. HRMS (ESI) calcd for C₂₈₅H₄₆₃N₅₀O₆₅ [M+3H]³+1876.4854, found 1876.4851; calcd for C₂₈₅H₄₆₄N₅₀O₆₅ [M+4H]⁴+1407.6160, found 1407.6158.

Compound 27: Lindlar catalyst (130 mg, 5% by wt.) was added to a stirred solution of azide 26 (180 mg, 0.03 mmol) in methanol (10 mL). The reaction flask was evacuated and flushed with hydrogen gas. The reaction mixture was stirred under hydrogen atmosphere (balloon) at room temperature for 5 h. After completion of the reaction, the catalyst was filtered through a pad of Celite, the cake was washed with methanol (10 mL) and the filtrate was concentrated under reduced pressure. The residue was purified by preparative HPLC using Welch Ultimate XB-C18 column (eluents: A=0.1% TFA in water, B=MeCN) to yield the compound 27 (130 mg, 74%) as pale yellow solid. HRMS (ESI) calcd for C₂₈₅H₄₆₅N₄₈O₆₅ [M+3H]³+1867.8219, found 1867.8217; calcd C₂₈₅H₄₆₆N₄₈O₆₅ [M+4H]⁴+1401.1184, found 1401.1181.

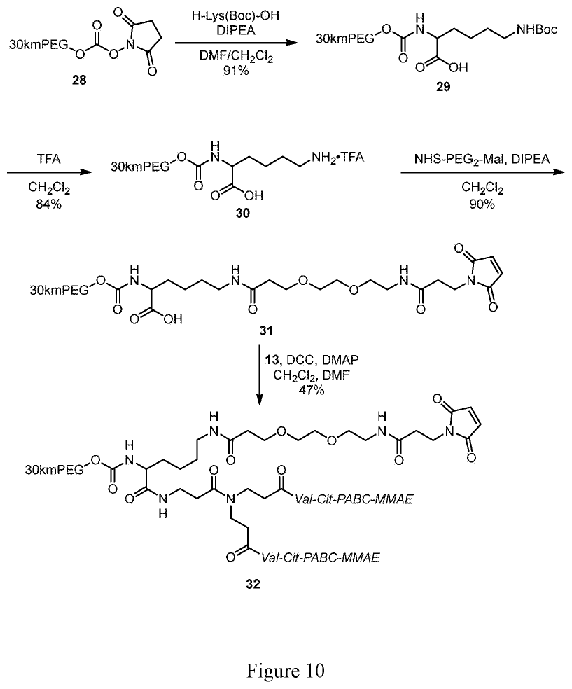

### Example 9. Preparation of Compound 32 (30 kmPEG(Maleimide)-2MMAE) (FIG. 10)

Compound 29: H-Lys(boc)-OH (369 mg, 1.5 mmol) was added into anhydrous DMF (100 mL) followed by addition of DIPEA (0.83 mL, 5.0 mmol), compound 28 (15 g, 0.5 mmol) and anhydrous CH₂C12(150 mL). The mixture was stirred under argon at room temperature overnight. The insoluble materials were filtered off. The solvent was removed and the residue was recrystallized from CH₂Cl₂/methyl tert-butyl ether (45 mL/300 mL). The isolated solids were recrystallized from MeCN/2-propanol (30 mL/450 mL). The product was dried at 40° C. over 4 h under vacuum to give the compound 29 (13.6 g, 91%) as white powder. 13C-NMR (126 MHz, CDCl3) δ 172.74, 155.65, 155.55, 78.41, 70.13 (PEG), 63.66, 58.55, 52.99, 39.90, 31.70, 29.17, 28.08, 21.97.

Compound 30: TFA (29.5 mL) was added to a solution of compound 29 (5.7 g, 0.19 mmol) in 57 ml anhydrous CH₂C12(57 mL). The mixture was stirred at room temperature for 1 h. Solvent was removed under vacuum as much as possible at <35° C. The residue was recrystallized from CH₂Cl₂/methyl tert-butyl ether (14.5 mL/115 mL) twice. The isolated product was dried under vacuum at 40° C. to yield the compound 30 (4.7 g, 84%) as white powder.

Compound 31: DIPEA (473 mg, 3.6 mmol) was added to a stirred solution of compound 30 (5.5 g, 0.18 mmol) in anhydrous CH₂Cl₂ (55 mL) at 0° C., followed by addition of NHS-PEG₂-Mal (0.2 g, 0.47 mmol). The mixture was stirred at 0° C. for 1.5 h. The solution was allowed to warm up slowly from 0° C. to room temperature and then stirred under argon atmosphere overnight. Solvent was removed and the residue was recrystallized from CH₂Cl₂/methyl tert-butyl ether (13.8 mL/110 mL). The isolated solids were recrystallized again from MeCN/2-propanol (11 mL/165 mL). The solids were dried under vacuum to yield the compound 31 (5.0 g, 90%) as white powder. 13C-NMR (126 MHz, CDCl3) δ 172.76, 171.46, 170.01, 169.94, 155.55, 133.82, 71.37, 70.01 (PEG), 69.05, 68.92, 66.49, 63.53, 58.44, 52.92, 38.65, 36.01, 33.84, 33.71, 31.36, 28.21, 21.85.

Compound 32: To a stirred solution of compound 31 (0.76 g, 0.025 mmol) in a mixture solvent of DMF/CH₂Cl₂ (5 mL/5 mL) at room temperature under argon, compound 13 (0.12 g, 0.05 mmol), DCC (31 mg, 0.15 mmol) and DMAP (28 mg, 0.23 mmol) were added. The reaction mixture was stirred at room temperature and monitored by HPLC. After the reaction was completed, the mixture was concentrated with Rotavapor. The residue was purified by preparative HPLC using Phenomenex Jupiter® C18 column (eluents: A=0.1% TFA in water, B=MeCN) to yield the compound 32(0.36 g, 47%) as white solid. MS (MALDI-TOF) m/z 33863.

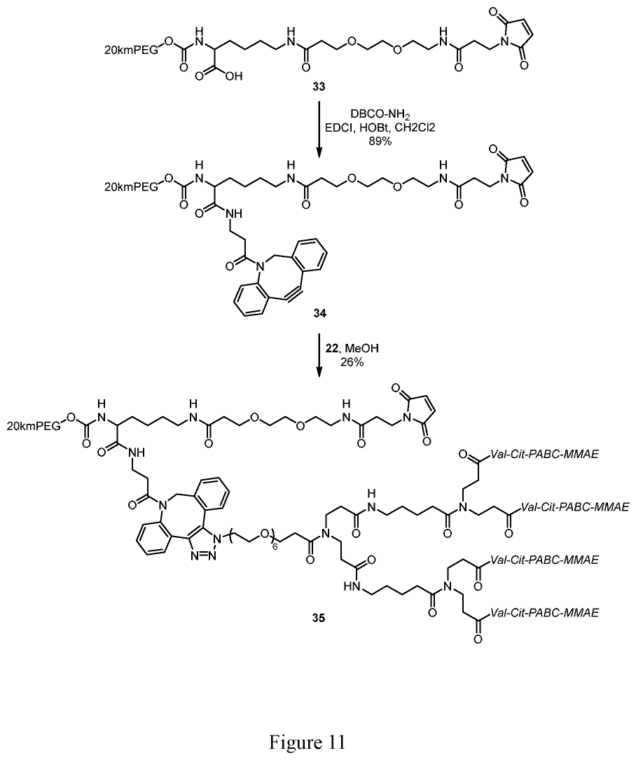

### Example 10. Preparation of Compound 35 (20 kmPEG(Maleimide)-4MMAE) (FIG. 11)

Compound 33: For synthesis of compound 33, refers to the preparation of compound 31. Compound 34: To a stirred solution of compound 33 (2.0 g, 0.1 mmol) in anhydrous CH₂Cl₂ (20 mL) at room temperature under argon, DBCO-NH₂ (83 mg, 0.3 mmol), EDCI (115 mg, 0.6 mmol) and HOBt (122 mg, 0.9 mmol) were added. The mixture was stirred at room temperature and monitored by HPLC. The solvent was removed and the residue was recrystallized from CH₂Cl₂/ methyl tert-butyl ether (5 mL/40 mL). The isolated solids were recrystallized again from MeCN/2-propanol (4 mL/60 mL). The solids were dried at 40° C. over 4 h under vacuum to give the compound 34 (1.9 g, 89%) as white powder. 13C-NMR (214 MHz, CDCl3) δ 171.12, 171.08, 170.05, 169.75, 155.64, 150.59 (d, J=21.4 Hz), 147.54 (d, J=6.6 Hz), 133.82, 131.69 (d, J=13.8 Hz), 128.70, 128.27 (d, J=11.3 Hz), 127.93 (d, J=5.4 Hz), 127.79, 127.39 (d, J=8.3 Hz), 126.66, 125.04 (d, J=6.0 Hz), 122.46 (d, J=4.9 Hz), 121.85 (d, J=11.3 Hz), 114.21 (d, J=9.8 Hz), 107.38 (d, J=33.6 Hz), 70.06 (PEG), 66.59, 63.64 (d, J=7.3 Hz), 58.50, 54.97 (d, J=13.3 Hz), 54.23 (d, J=59.1 Hz), 38.61, 38.40, 36.31, 34.86 (d, J=18.0 Hz), 34.05 (d, J=20.8 Hz), 33.89, 33.78, 31.76 (d, J=40.2 Hz), 28.31 (d, J=9.8 Hz), 21.99 (d, J=17.1 Hz).

Compound 35: Compound 34 (147 mg, 0.007 mmol) was dissolved in anhydrous MeOH (3 mL), followed by addition of compound 22 (40 mg, 0.007 mmol). The reaction mixture was stirred at room temperature for 24 h. The mixture was concentrated with Rotavapor and the residue was purified by preparative HPLC using Phenomenex Jupiter® C18 column (eluents: A=0.1% TFA in water, B=MeCN) to yield the compound 35 (41 mg, 22%) as white solid. MS (MALDI-TOF) m/z 25963.

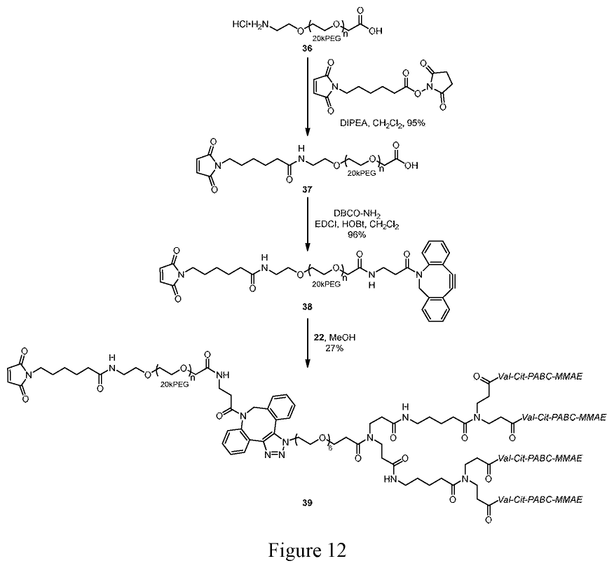

### Example 11. Preparation of Compound 39 (Maleimide-20 mPEG-4MMAE) (FIG. 12)

Compound 37: To a stirred solution of amine-PEG20k-CO₂H (36) (1.0 g, 0.05 mmol) in anhydrous CH₂Cl₂ (10 mL) at 0° C., DIPEA (83 μL, 0.5 mmol) was added followed by addition of 6-maleimidohexanoic acid N-hydroxysuccinimide ester (46 mg, 0.15 mmol). The mixture was stirred at 0° C. for 1.5 h. The solution was allowed to warm up slowly from 0° C. to room temperature and stirred under argon atmosphere overnight. Solvent was removed and the residue was recrystallized from CH₂Cl₂/ methyl tert-butyl ether (2.5 mL/20 mL). The isolated solids were recrystallized again from MeCN/2-propanol (2 mL/30 mL). The residue was dried under vacuum to yield the compound 37 (0.95 g, 95%) as white powder.

Compound 38: To a stirred solution of compound 37 (0.9 g, 0.045 mmol) in anhydrous CH₂Cl₂ (9 mL) at room temperature under argon, DBCO-NH₂ (37 mg, 0.14 mmol), EDCI (52 mg, 0.27 mmol) and HOBt (55 mg, 0.41 mmol) were added. The mixture was stirred at room temperature and monitored by HPLC. The solvent was removed and the residue was recrystallized from CH₂Cl₂/ methyl tert-butyl ether (2.5 mL/20 mL). The isolated solids were recrystallized again from MeCN/2-propanol (2 mL/30 mL). The product was dried at 40° C. over 4 h under vacuum to give the compound 38 (0.86 g, 89%) as white powder.

Compound 39: Compound 38 (166 mg, 0.008 mmol) was dissolved in anhydrous MeOH (3 mL), followed by addition of compound 22 (30 mg, 0.006 mmol). The reaction mixture was stirred at room temperature for 24 h. The solvent was removed with Rotavapor and the residue was purified by preparative HPLC using Phenomenex Jupiter® C18 column (eluents: A=0.1% TFA in water, B=MeCN) to yield the compound 39 (37 mg, 27%) as white solid. HRMS (ESI) or NMR

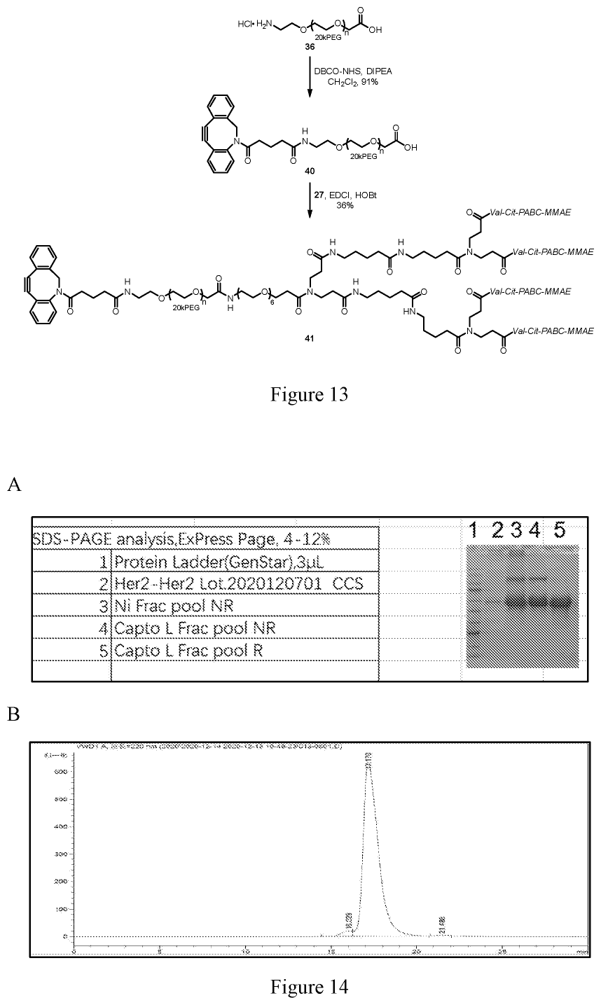

### Example 12. Preparation of Compound 41 (DBCO-20 kPEG-4MMAE) (FIG. 13)

Compound 40: To a stirred solution of amine-PEG20k-CO₂H (36) (1.0 g, 0.05 mmol) in anhydrous CH₂Cl₂ (10 mL) at 0° C., DIPEA (83 μL, 0.5 mmol) was added followed by addition of DBCO-NHS (60 mg, 0.15 mmol). The mixture was stirred at 0° C. for 1.5 h. The solution was allowed to warm up slowly from 0° C. to room temperature and stirred under argon atmosphere overnight. Solvent was removed and the residue was recrystallized from CH₂Cl₂/ methyl tert-butyl ether (2.5 mL/20 mL). The isolated solids were recrystallized again from MeCN/2-propanol (2 mL/30 mL). The residue was dried under vacuum to yield the compound 40 (0.91 g, 91%) as white powder.

Compound 41: Under argon atmosphere, the compound 40 (120 mg, 0.006 mmol) was dissolved in a mixture solvent of CH₂Cl₂/DMF (2 mL/2 mL) followed by the addition of compound 27 (50 mg, 0.009 mmol), EDCI (6.9 mg, 0.036 mmol) and HOBt (7.3 mg, 0.054 mmol) successively. The resulting reaction mixture was stirred at room temperature for 24 h. The reaction mixture was concentrated with Rotavapor and the residue was purified by preparative HPLC using Phenomenex Jupiter® C18 column (eluents: A=0.1% TFA in water, B=MeCN) to yield the compound 41 (53 mg, 36%) as white solid. MS (MALDI-TOF) m z 25450 Da.

### Example 13. Preparation of SCAHer2II×SCAHer2IV (Compound 42) (FIG. 14)

SCAHer2II×SCAHer2IV with the amino acid sequence of SEQ ID NO: 1 was prepared and purified as described in Example 2. In particular, about 1.6 L of supernatant of culture media of host cells expressing SCAHer2II×SCAHer2IV was collected after centrifugation and loaded to a Ni-charged column (2.6 cm×13 cm) (Cat #AA207311, BestChrome, Shanghai, China) pre-equilibrated with 50 mM sodium phosphate, 100 mM NaCl, pH7.0. The protein was eluted off with a buffer of 50 mM sodium phosphate, 250 mM imidazole, 100 mM NaCl, pH7.0 and fractionated in 15 mL tubes. Resulted 82 mg of captured protein was further purified with a CaptoL column (Cat #17-5478-02, GE Healthcare, NJ). CaptoL column (1.6 cm×8 cm) was pre-equilibrated with 50 mM sodium phosphate, 100 mM NaCl, pH7.0, and protein was eluted with 75 mM acetic acid, pH 3.0, resulting 58.3 mg of protein. FIG. 14 showed SDS-PAGE and SEC-HPLC analysis of purified compound 42 (SCAHer2II×SCAHer2IV).

### Example 14. Preparation of 30 kmPEG-(SCAHer2II×SCAHer2IV)-2MMAE (Compound

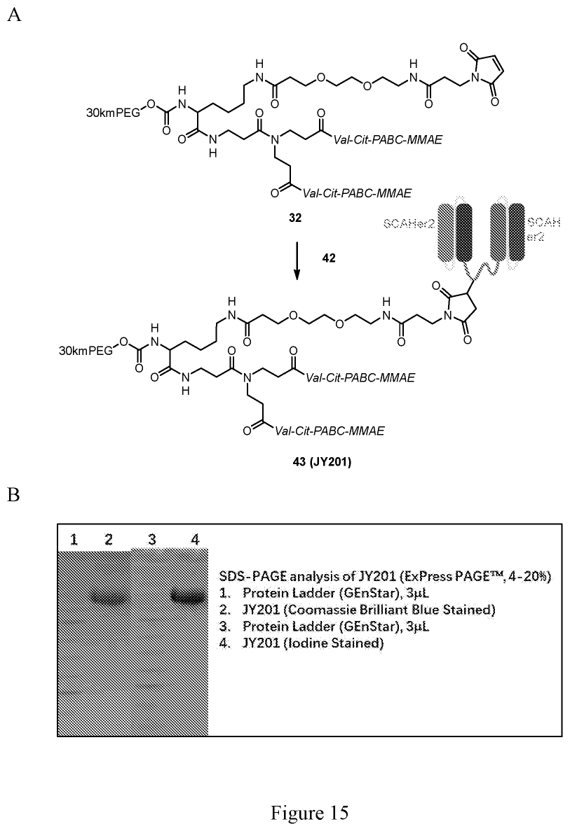

43, JY201) (FIG. 15) Protein SCAHer2II×SCAHer2IV 42 was treated by reducing agent TCEP-HCl in PBS buffer (pH=7.4) at room temperature for 30 min, and then the pH was adjusted to 6.8 with a pH=4.12 stock solution of 500 mM sodium phosphate. The treated protein was concentrated to 5 mg/mL before conjugation. Conjugation of SCAHer2II×SCAHer2IV was conducted at room temperature for 3 hours with 5 to 10 mole equivalent of compound 32 (30 kmPEG(Maleimide)-2MMAE. The reaction was quenched with 10 mM of L-cystine at room temperature for 10 min. Final product 30 kmPEG-(SCAHer2II×SCAHer2IV)-2MMAE, JY201, was purified with cation exchange chromatography column (CM Fast Flow, Cat #17-0719-01, GE Healthcare, NJ) at pH 6.5 in 20 mM phosphate buffer. FIG. 15A schematically illustrates the reaction scheme of preparing compound 43, and the resulting compound 43 was confirmed by SDS-PAGE (FIG. 15B).

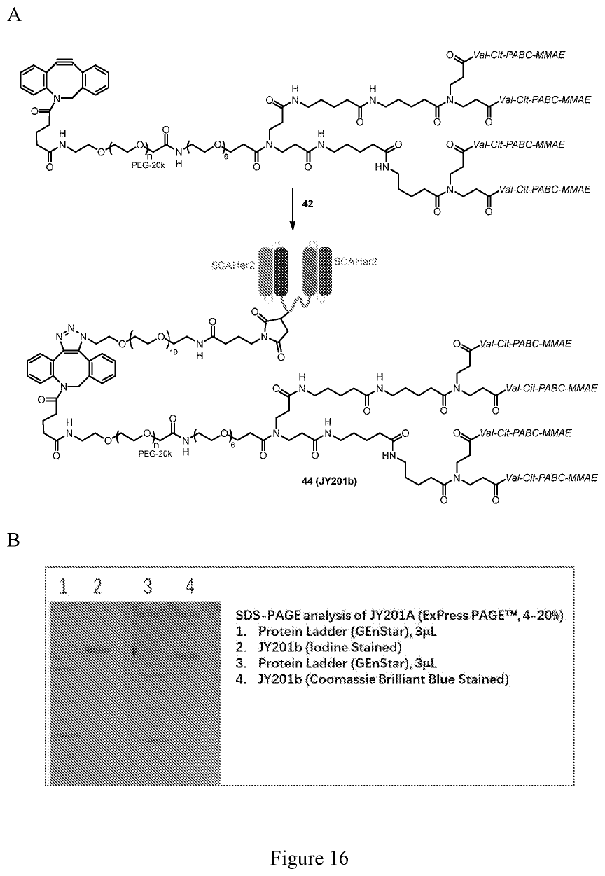

### Example 15. Preparation of SCAHer2II×SCAHer2IV-20 kPEG-4MMAE (Compound 44, JY201b) (FIG. 16)

Protein SCAHer2II×SCAHer2IV 42 was treated by reducing agent TCEP-HCl in PBS buffer (pH=7.4) at room temperature for 30 min, and then the pH was adjusted to 6.8 with a pH=4.12 stock solution of 500 mM sodium phosphate. The treated protein was concentrated to 5 mg/mL before conjugation. Conjugation of SCAHer2II×SCAHer2IV was conducted at room temperature for 3 hours with 5 to 10 mole equivalent of compound 41 (DBCO-20 kPEG-4MMAE). The reaction was quenched with 10 mM of L-cystine at room temperature for 10 min. Final product was purified with size-exclusion chromatography column, HiPrep™ 16/60, Sephacryl™ S-300HR (Cat #17-1167-01, GE Healthcare, NJ) at pH 6.5 in 20 mM phosphate buffer. FIG. 16A schematically illustrates the reaction scheme of preparing compound 44 (SCAHer2II×SCAHer2IV-20 kPEG-4MMAE, JY201b), and the final compound 44 was confirmed by SDS-PAGE (FIG. 16B).

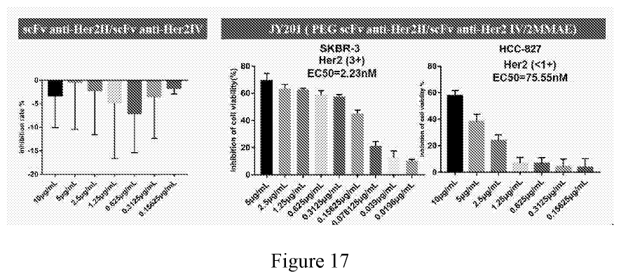

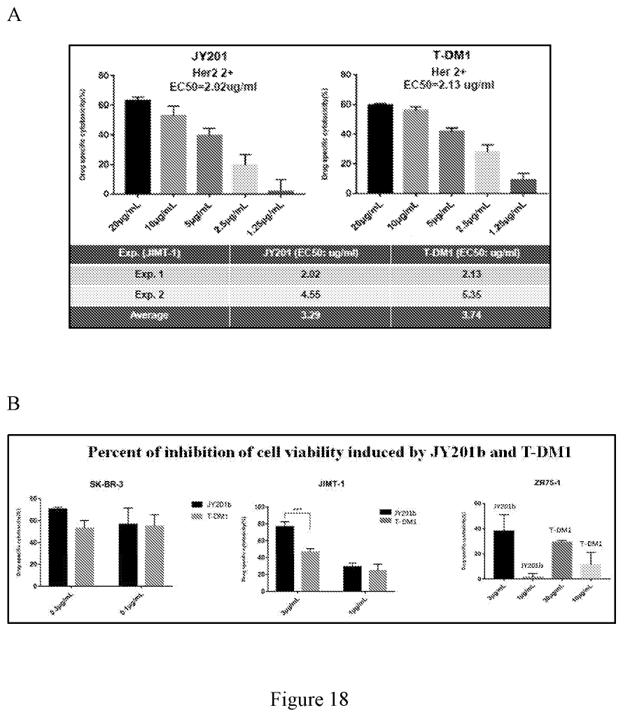

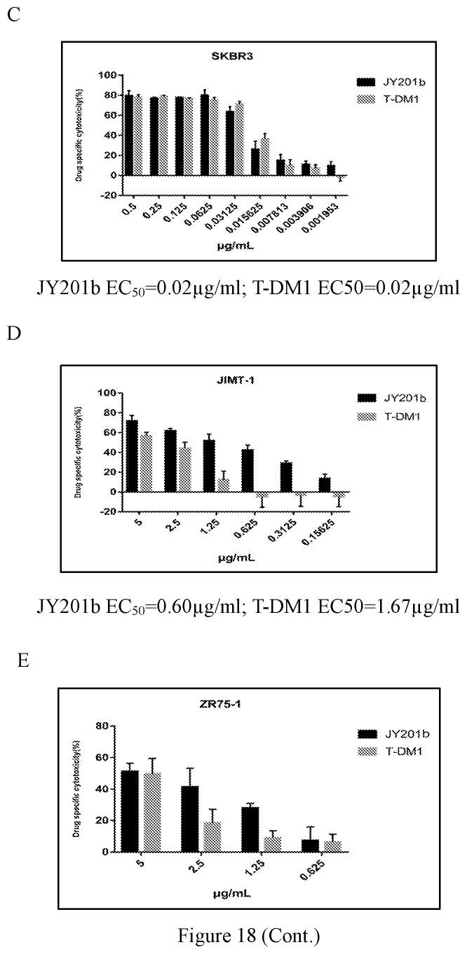

### Example 16. In Vitro Cytotoxicity of Compound 43 (JY201) and Compound 44 (JY201b) (FIGS. 17, 18)

In order to assess the effect of PEGylation on in-vitro cytotoxicity of the PEGylated BsADC JY201 and JY201b, cell viability assay was performed after incubation of the cells with Compound 43 (JY201) or Compound 44 (JY201b), or controls. In particular, 4×10⁴ cells/well were seeded in a flat-bottom 96-well plate to allow cells to adhere. After 6h, cells were treated with indicated doses of JY201 at 37° C. for 72 hours, followed by addition of 20 μl MTS to each well according to manufacturer's protocol. Absorbance at OD₄₉₀ nm was then detected and the percentage of cytotoxicity was calculated.

FIG. 17 showed that EC50s of JY201 for SKBR-3 and for HCC-827 cells were 2.23 nM and 75.55 nM respectively. Since HCC827 cells expressed a much lower level of Her2 than SKBR-3 (Kayatani, H. et al. 2020, Biochem Biophys Res Commun 532, 341-346), these results demonstrated that JY201 can induce potent cytotoxicity to tumor cells with very low Her2 expression. Moreover, the result from left panel of FIG. 17 indicated that the single chain antibody Her2II×Her2IV did not induce detectable toxicity to SKBR-3, and thus the cytotoxicity of JY201 was caused by the payload MMAE.

Using the same method described above, the in vitro cytotoxicity of JY201 on JIMT-1 cells was tested and compared with Trastuzumab emtansine (T-DM1). Surprisingly, the results from FIG. 18A showed that EC50 for JY201 was very similar to that for T-DM1 (3.29 μg/ml and 3.74 μg/ml, respectively) although the DAR (Drug to Antibody Ratio) was only 2 for JY201 while DAR is 4 for T-DM1. These results demonstrated that the potency of PEGylated BsADC JY201 with only 2 payloads was comparable with T-DM1 with 4 payloads in inducing in vitro cytotoxicity to tumor cells.

Further experiments were conducted to test the in vitro cytotoxicity of JY201b (with a DAR of 4) and compared with JY201 and T-DM1 (FIGS. 18B, C, D and E). The results showed that PEGylated BsADC JY201b with 4 payloads was more potent than JY201 with 2 payloads (FIGS. 18A and D) and comparable or more potent than T-DM1 across the panel of tumor cell lines at the indicated concentration tested. It is worth noting that at the low end subset of concentrations of tested samples, JY201b performed much better than T-DM1 in inducing potent cytotoxicity to tumor cells with low expression of target antigens (Her2 expression level: SKBR-3>JIMT-1>ZR75-1, see following table). This merit, together with better toxic profile, provides great hope for JY201b to treat cancer patients with low expression of Her2, to whom current therapies are not available.

| Cells | Her2 expression |
|---|---|
| SKBR-3 | >3+ |
| JIMT-1 | 2+ |
| ZR75-1 | 1+ |

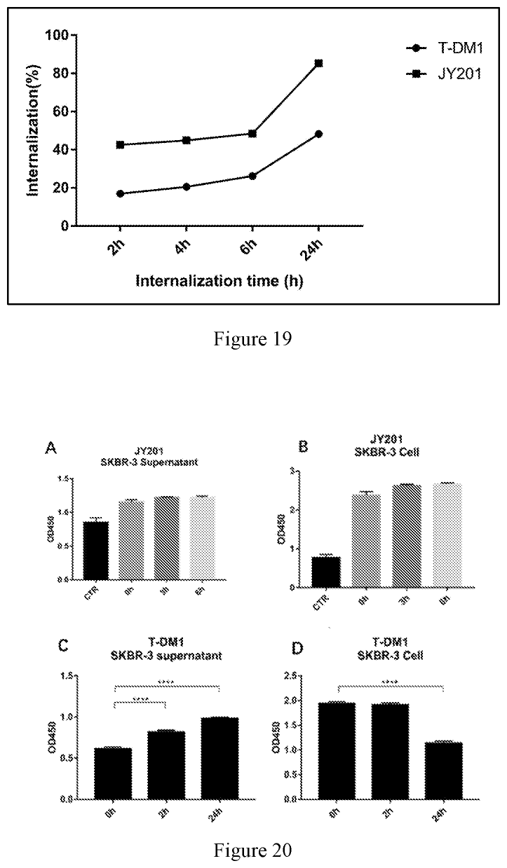

### Example 17. Internalization of JY201 by Target Cells (FIG. 19)

To investigate the mechanism of the cytotoxic effect demonstrated in Example 16, the internalization of PEGylated BsADC JY201 by SKBR-3 cells was examined with a flow cytometry method described by Matsuzaki (Matsuzaki, S. et al. 2018, International Journal of Cancer 142, 1056-1066). After trypsinization, SKBR-3 cells were washed and resuspended to a concentration of 1×10⁷/mL by PBS containing 2% FBS. The cell suspensions were aliquoted at 100 μl/tube. SKBR-3 cells were treated with 10 μg/ml Flour647 labeled T-DM1 or JY201 at 4° C. overnight. After washed with pre-cooled PBS twice, the cells were incubated at 37° C. for indicated time period to allow T-DM1 and JY201 internalized. The cells incubated were washed with 3×200 μl FACS buffer. After the final wash, 100 ul FACS buffer was added to resuspend the cells for flow cytometry analysis. Internalization rate was calculated using the formular:

(Total MFI at 4° C.−Total MFI at 37° C.)/Total MFI at 4° C.×100%.

Results from FIG. 19 showed that the internalization rates of JY201 by SKBR-3 cells were about 2× higher than T-DM1 at all time points tested, although the affinity of JY201 to the target was much weaker than T-DM1 (data not shown). This result implied that dynamic internalization and efflux mechanism adopt by traditional Fc bearing ADC may not apply to the PEGylated ADCs disclosed herein. Of note, the internalization mechanisms associated with binding of Fc components of traditional ADCs to, for example, FcγR or mannose receptor on the normal tissues or cells often contribute to off-target toxicity or even dose-limiting toxicity of the ADC drugs (Krop I E, et. al. J Clin Oncol, 30, 3234-41, 2012; Uppal, H. et al. 2015, Clin Cancer Res 21, 123-133; Gorovits, B. e. al. 2013, Cancer Immunol Immunother 62, 217-223).

### Example 18. No Efflux Out of Target Cells after Internalization of JY201 (FIG. 20)

Efflux out of target cells after internalization was commonly seen for Fc-bearing Adcs. This could result in off-target toxicity, decreased efficacy and drug resistance. This phenomenon has been attributed to the FcRn mediated recycling (Junghans, R. P. et. al. 1996, Proc Natl Acad Sci USA 93, 5512-5516; Ryman, J. T. et. al. 2017, CPT Pharmacometrics Syst Pharmacol 6, 576-588). It was reported that 50% internalized Trastuzumab flowed out of target cells within 5 minutes of internalization, and this number increased to 85% within 30 minutes of internalization (Barok, M., Joensuu, et. al. 2014, Breast Cancer Res 16, 209-209).

To examine whether JY201 flows out of the target cells after internalization, HRP (horseradish peroxidase) was conjugated to JY201 by following the manufacturer provided protocol. 3×10⁴ SK-BR3 cells were seeded in a flat-bottom 96-well plate overnight to allow cells to adhere. On the second day, cells were washed and incubated with 0.25 ug/mL of JY201 for 18 hours at room temperature. After 3× washing by complete medium, the cells were further incubated at 37° C. Cell lysates and supernatants were collected at different time points. The content of JY201-HRP in cell lysate and cell supernatant was tested by adding 50 μl/well TMB (3,3′,5,5′-tetramethylbenzidine) solution. OD450 was obtained on a microplate analyzer after the reaction was terminated by 50 μl/well 0.2 M sulphuric acid. The same experiment was performed for T-DM1 in that 0.25 g/ml T-DM1-HRP was incubated with the cells for 4 hours followed by washing, medium change and further incubation for 2h and 24h.

FIG. 20A showed that, comparing to 0 hr, JY201 in the supernatant did not increase significantly by further incubation for 3 hrs and 6 hrs. Meanwhile, JY201 inside the cell lysates did not decrease at 3 hrs and 6 hrs (FIG. 20B). Further, the OD450 of the cell lysates at 0 hr was at least 2 times higher than that of the supernatant. It can be seen that JY201 was internalized and the internalized JY201 did not flow out to the supernatant.

For comparison, the levels of T-DM1 in the supernatant was also measured, which increased significantly (p<0.001) after 2 hrs incubation (FIG. 20C). In addition, the level of T-DM1 after 24 hrs incubation was significantly higher than 2 hrs incubation, indicating continuing efflux of T-DM1. Consistently, T-DM1 in the cell lysates decreased significantly at 24 hrs (p<0.001) (FIG. 20D). The efflux mechanism of T-DM1 could result in reduced clinical efficacy and increased toxicity of the drug.

Overall, data from FIG. 20 demonstrates surprising results of no recycling or efflux mechanism for JY201, which was probably due to the lack of Fc component of JY201.

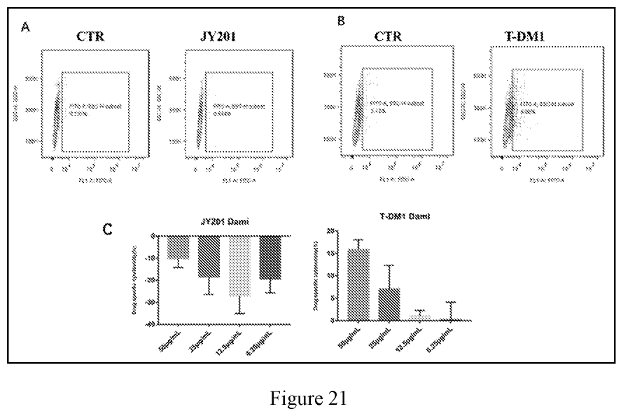

### Example 19. JY201 Shows No Cytotoxicity to Megakaryocytes (FIG. 21)

Thrombocytopenia characterized by low platelet counts is a major adverse event in cancer patients treated with ADCs (Uppal, H. et al. 2015, Clin Cancer Res 21, 123-133; Donaghy, H. 2016, MAbs 8, 659-671; de Goeij, B. E. et. al. 2016, Curr Opin Immunol 40, 14-23), which is responsible for dose-limiting toxicity of T-DM1 ((Krop I E, et. al. J Clin Oncol, 30, 3234-41, 2012)). To examine the cytotoxicity of JY201 in relation to thrombocytopenia, the binding and cytotoxicity of JY102 to DAMI (a cell line of megakaryocytes which are the parental cells for the terminal differentiated platelets (Lev, P. R. et al. 2011, Platelets 22, 28-38)) were tested.

For binding experiment, DAMI cells were collected and resuspend to a concentration of approximately 5×10⁶ cells/ml in ice cold PBS containing 2% FBS. The cells were then incubated with JY201 or controls and subjected to flow cytometry analysis by using the same method described in example 17. The same method described in example 16 was used to evaluate the in vitro cytotoxicity to DAMI cells.

The result shown in FIG. 21C demonstrated that PEGylated BsADC JY201 surprisingly did not induce cytotoxicity to DAMI cells even at the high concentration of 50 g/ml, while T-DM1 induced significant drug-specific cytotoxicity at the tested concentrations. The unexpected result is consistent with the results in FIGS. 21A and 21B, in which FITC labelled T-DM1 bond to DAMI cells while JY201 did not, probably because of the absence of the Fc regions.

In summary, current data implicate that the cytotoxicity of JY201 is tissue specific, exerting cytotoxicity only to tumor cells but not megakaryocytes. The unexpected and superior properties of JY201 yield great opportunities to address some major adverse events caused by ADC-induced thrombocytopenia and others in clinical.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims.

1. A compound of the Formula (Ib) 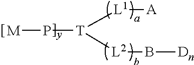
wherein
P is a non-immunogenic polymer;
M is H or a terminal capping group selected from C₁₋₅₀ alkyl and aryl, wherein one or more carbons of said alkyl are optionally replaced with a heteroatom;
y is an integer selected from 1 to 10;
A is an antibody or an antigen binding fragment thereof, and
T is a multifunctional small molecule linker moiety;
each of L¹ and L² is independently a hetero or homobifunctional linker;
each of a and b is an integer selected from 0-10;
B is a branched linker, wherein each branch has an amino acid sequence or carbohydrate moiety linked to a self-immolating spacer, wherein cleavage of the amino acid sequence or carbohydrate moiety by an enzyme triggers self-immolating mechanism to release D, or each branch has a disulfide bond or a cleavable bond, wherein cleavage of the disulfide bond or the cleavable bond releases D or its derivative;
each of D is independently a cytotoxic small molecule or peptide; and
n is an integer selected from 1-25.

2. The compound of claim 1, wherein T is a tri-functional linker derived from a molecule with three functional groups independently selected from hydroxyl, amino, hydrazinyl, azide, alkene, alkyne, carboxyl (aldehyde, ketone, ester, carboxylic acid, anhydride, acyl halide), thiol, disulfide, nitrile, epoxide, imine, nitro and halide, and wherein the linkage between T and (L¹)ₐ and the linkage between T and (L²)_{b} are the same or different.

3. The compound of claim 2, wherein T is lysine or is derived from lysine.

4. The compound of claim 1, wherein the functional group at the linker terminal of L¹ is capable of site-specific conjugation with A, and is selected from the group consisting of thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and Iodine.

5. The compound of claim 1, wherein the antibody is a mono-specific or multi-specific full length antibody, a single chain antibody, a nanobody, or an antigen binding domain thereof.

6-9. (canceled)

10. The compound of claim 1, wherein the antibody is a bispecific antibody, wherein the two binding domains of the bispecific antibody bind to the same tumor associated antigen (TAA), bind to two different TAAs, or bind to a TAA and an antigen expressed on T cells or NK cells.

11. (canceled)

12. The compound of claim 10, wherein the antibody is an anti-Her2II x_anti-Her2IV single chain bispecific antibody.

13. The compound of claim 1, wherein the antibody has an amino acid sequence as shown in SEQ ID NO: 1 or SEQ ID NO: 2.

14. (canceled)

15. The compound of claim 10, wherein the two binding domains of the bispecific single chain antibody are linked via a linker, and wherein the linker comprises a cysteine or an unnatural amino acid residue for site-specific conjugation of the antibody to L¹.

16. The compound of claim 15, wherein the unnatural amino acid is selected from genetically-encoded alkene lysines (such as N₆-(hex-5-enoyl)-L-lysine), 2-Amino-8-oxononanoic acid, m or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino)hexanoic acid, azidohomoalanine, pyrrolysine analogue N6-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, NF-Acryloyl-1-lysine, NF-5-norbornene-2-yloxycarbonyl-1-lysine, N-ε-(Cyclooct-2-yn-1-yloxy)carbonyl)-L-lysine, N-ε-(2-(Cyclooct-2-yn-1-yloxy)ethyl) carbonyl-L-lysine, genetically encoded Tetrazine Amino Acid (such as 4-(6-methyl-s-tetrazin-3-yl)aminophenylalanine).

17. The compound of claim 1, wherein D is selected from a DNA crosslinker agent, a microtubule inhibitor, a DNA alkylator, a topoisomerase inhibitor or a combination thereof.

18. The compound of claim 17, wherein D is selected from MMAE, MMAF, SN38, DM1, DM4, calicheamycins, pyrrolobenzodiazepines, duocarmycins or a derivate thereof, or a combination thereof; or
wherein D is selected from Vinca alkaloid, laulimalide, taxane, colchicine, tubulysins, Cryptophycins, Hemiasterlin, Cemadotin, Rhizoxin, Discodermolide, taccalonolide A or B or AF or AJ, taccalonolide AI-epoxide, CA-4, epothilone A and B, laulimalide, paclitaxel, docetaxel, doxorubicin, Camptothecin, iSGD-1882, centanamycin, PNU-159682, uncialamycin, indolinobenzodiazepine dimers, β-amanitin, Amatoxins, thailanstatins or a derivate or analogous thereof, or a combination thereof.

19. (canceled)

20. The compound of claim 1, wherein the non-immunogenic polymer is polyethylene glycol (PEG).

21. The compound of claim 20 wherein the PEG is a liner PEG or a branched PEG, wherein at least one terminal of the polyethylene glycol is capped with methyl or a low molecule weight alkyl.

22. (canceled)

23. The compound of claim 20, wherein a total molecule weight of the PEG is from 100 to 80000.

24. The compound of claim 20, wherein the PEG is linked to the trifunctional or tetrafunctional or any other cyclic or noncyclic multifunctional moiety T through a permanent bond or a cleavable bond.

25. A compound of the Formula (Ic) 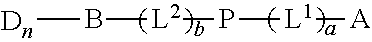
wherein
P is a liner PEG;
A is an antibody or an antigen binding fragment thereof,
each of L¹ and L² is independently a bifunctional linker;
each of a and b is an integer selected from 0-10;
B is a branched linker, wherein each branch has an amino acid sequence or carbohydrate moiety linked to a self-immolating spacer, wherein cleavage of the amino acid sequence or carbohydrate moiety by an enzyme triggers self-immolating mechanism to release D, or each branch has a disulfide bond or a cleavable bond, wherein cleavage of the disulfide bond or the cleavable bond releases D or its derivative;
each of D is independently a cytotoxic small molecule or peptide;
n is an integer selected from 1-25.

26-33. (canceled)

34. The compound of claim 25, wherein the antibody has an amino acid sequence as shown in SEQ ID NO: 1 or SEQ ID NO: 2.

35-41. (canceled)

42. The compound of claim 1, wherein each of L¹ and L² is independently selected from:
—(CH₂)ₐXY(CH₂)_{b}—,
—X(CH₂)ₐO(CH₂CH₂O)_{c}(CH₂)_{b}Y—,
—(CH₂)ₐheterocyclyl-,
—(CH₂)ₐX—,
—X(CH₂)ₐY—,
—W₁—(CH₂)ₐC(O)NR₁(CH₂)_{b}O(CH₂CH₂O)_{c}(CH₂)_{d}C(O)—,
—C(O)(CH₂)ₐO(CH₂CH₂O)_{b}(CH₂)_{c}W₂C(O)(CH₂)_{d}NR₁—,
—W₃—(CH₂)ₐC(O)NR₁(CH₂)_{b}O(CH₂CH₂O)_{c}(CH₂)_{d}W₂C(O)(CH₂)ₑC(O)—,
wherein a, b, c, d and e are each an integer independently selected from 0 to 25; each of X and Y is independently selected from C(═O), NR₁, S, O, CR₂R₃ or Null; R₁ and R₂ independently represent hydrogen, C₁₋₁₀ alkyl or (CH₂)₁₋₁₀C(═O); W₁ and/or W₃ is derived from a maleimido-based moiety and W₂ represents a triazolyl or a tetrazolyl containing group; the heterocyclyl group is selected from a maleimido-derived moiety or a tetrazolyl-based or a triazolyl-based moiety.

43. The compound of claim 1, wherein each of (L¹)ₐ and (L²)_{b} is independently selected from: 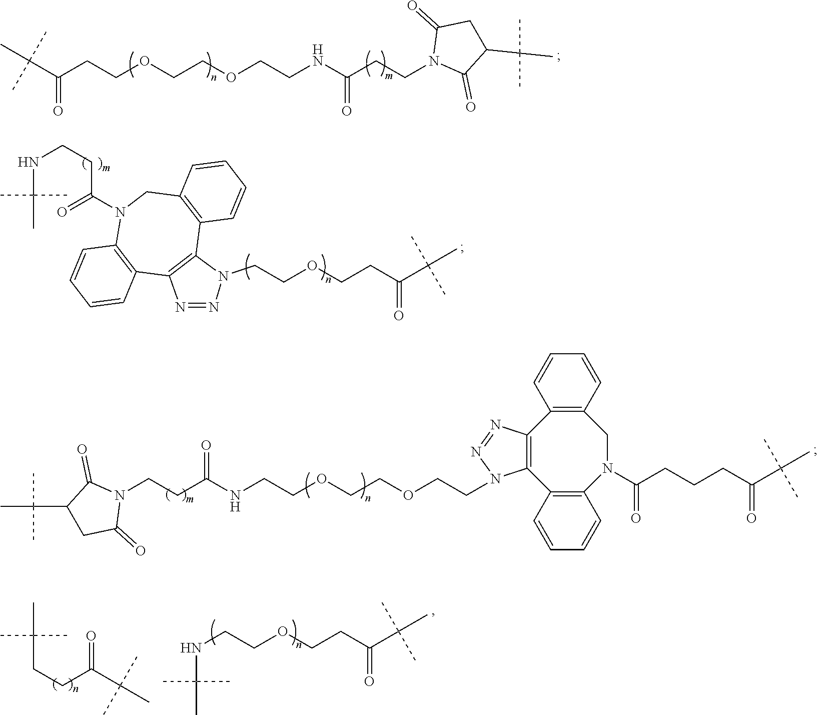
wherein n and m are integer and independently selected from 0 to 20.

44. The compound of claim 1, wherein the branch linker B comprise an extension spacer, a trigger unit, a self-immolating spacer or any combination thereof, wherein the trigger unit is an amino acid sequence or a β-glucoronide or μ-galactoside trigger moiety cleavable by an enzyme such as cathepsin B, plasmin, matrix metalloproteinases (MMPs), β-glucuronidases, β-galactosidases; or a pH liable linker that can release the drug D or its derivatives at acidic pH conditions, or a disulfide bond linker that can release the drug D or its derivatives by glutathione, thioredoxin family members (WCGH/PCK) or thio reductase.

45. The compound of claim 44, wherein the branch linker B is selected from 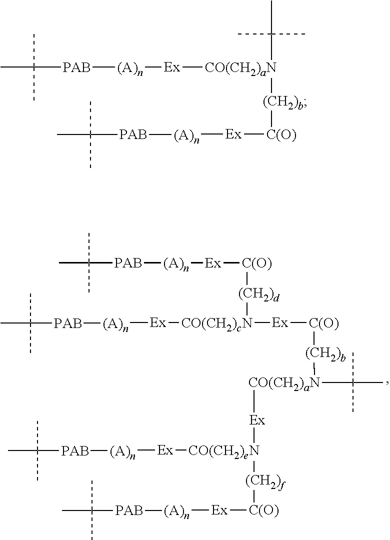
wherein:
a, b, c, d, e and f are each an integer and independently selected from 1-25;
(A)ₙ is a trigger unit of amino acid sequence such as Val-Cit, Val-Ala, Val-Lys, Phe-Lys, Phe-Cit, Phe-Arg, Phe-Ala, Ala-Lys, Leu-Cit, Ile-Cit, Trp-Cit, D-Phe-LPhe-Lys, Phe-Phe-Lys, D-Phe-Phe-Lys, Gly-Phe-Lys, Gly-Phe-Leu-Gly, or Ala-Leu-Ala-Leu;
PAB is para-aminobenzyl alcohol;
each of Ex is an extension spacer comprising a linker chain that is independently selected from:
—NR¹(CH₂)ₓO(CH₂CH₂O)_{y}(CH₂)_{z}C(O)—,
—C(O)(CH₂)ₓNR¹—,
—NR¹(CH₂)ₓO(CH₂CH₂O)_{y}(CH₂)_{z}NR²,
—NR¹(CH₂)ₓNR²,
—NR¹(CH₂)ₓO(CH₂CH₂O)_{y}(CH₂)_{z}O—,
—O(CH₂)ₓNR¹—,
—C(O)(CH₂)ₓO—,
—O(CH₂)ₓO(CH₂CH₂O)_{y}(CH₂)_{z}C(O)—,
—C(O)(CH₂)ₓO(CH₂CH₂O)_{y}(CH₂)_{z}C(O)—,
—C(O)(CH₂)ₓC(O)—,
or Null,
wherein x, y, and z are each an integer and independently selected from 0 to 25; and R¹ and R² independently represent hydrogen or a C₁₋₁₀ alkyl group.

46. The compound of claim 1, wherein the branch linker B is selected from

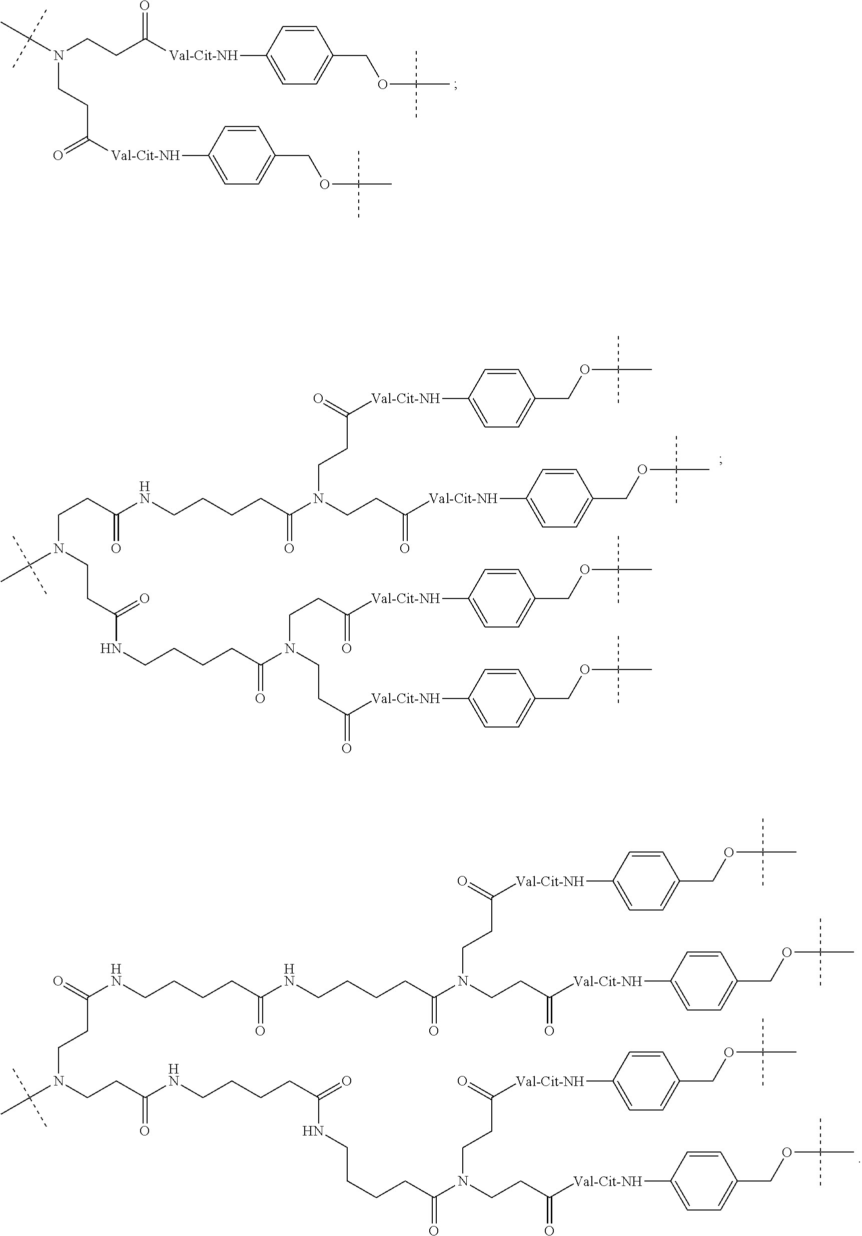

47. The compound of claim 1 selected from the formula: 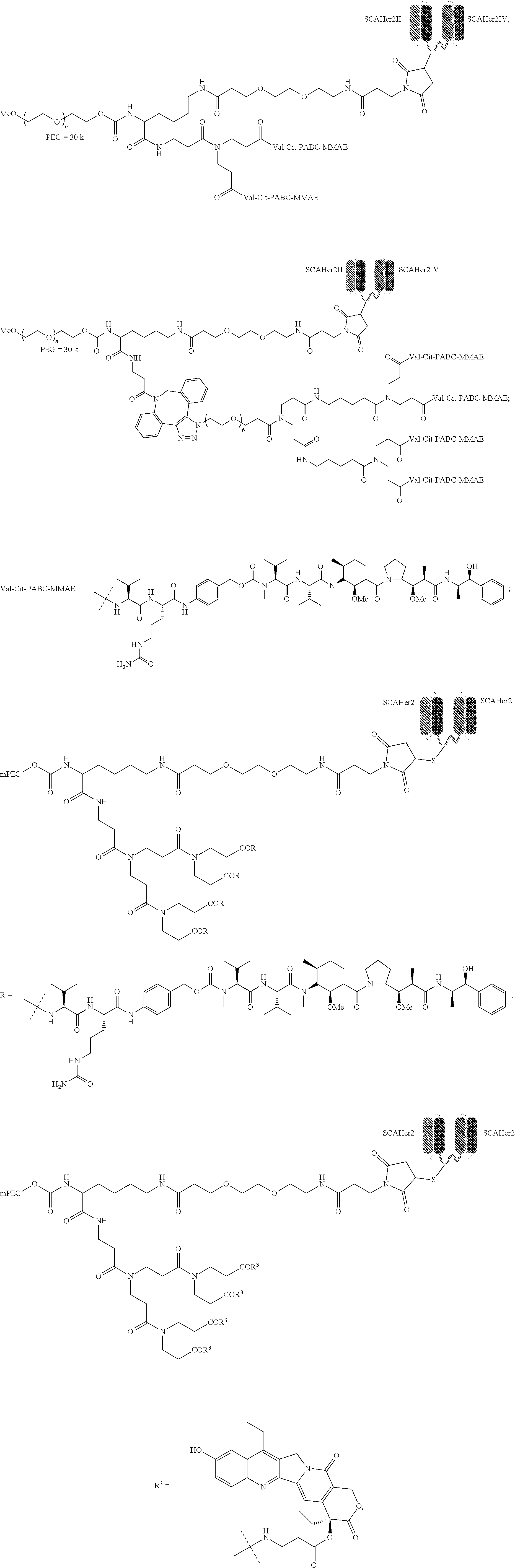
or a pharmaceutically acceptable salt thereof.

48. The compound of claim 25 selected from the formula:

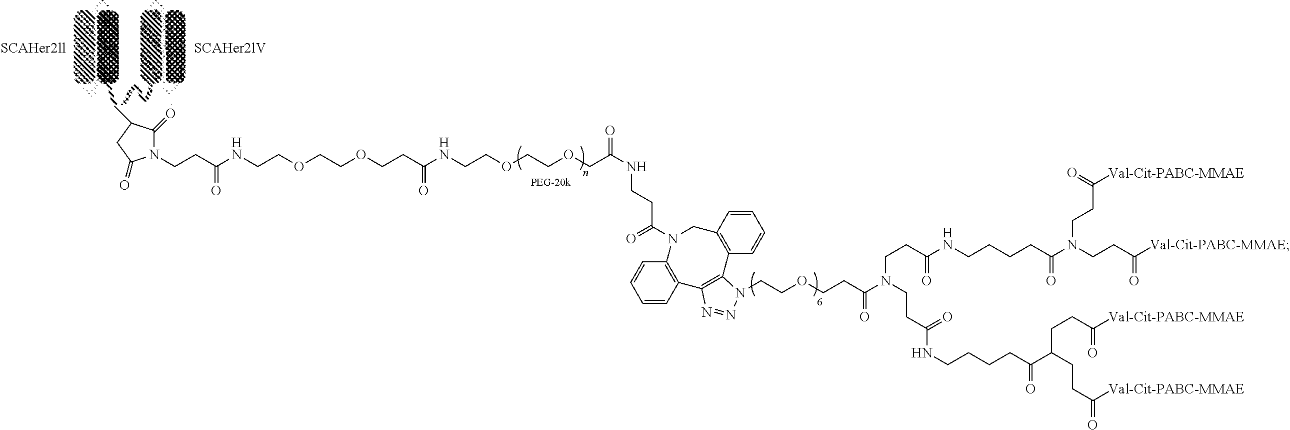

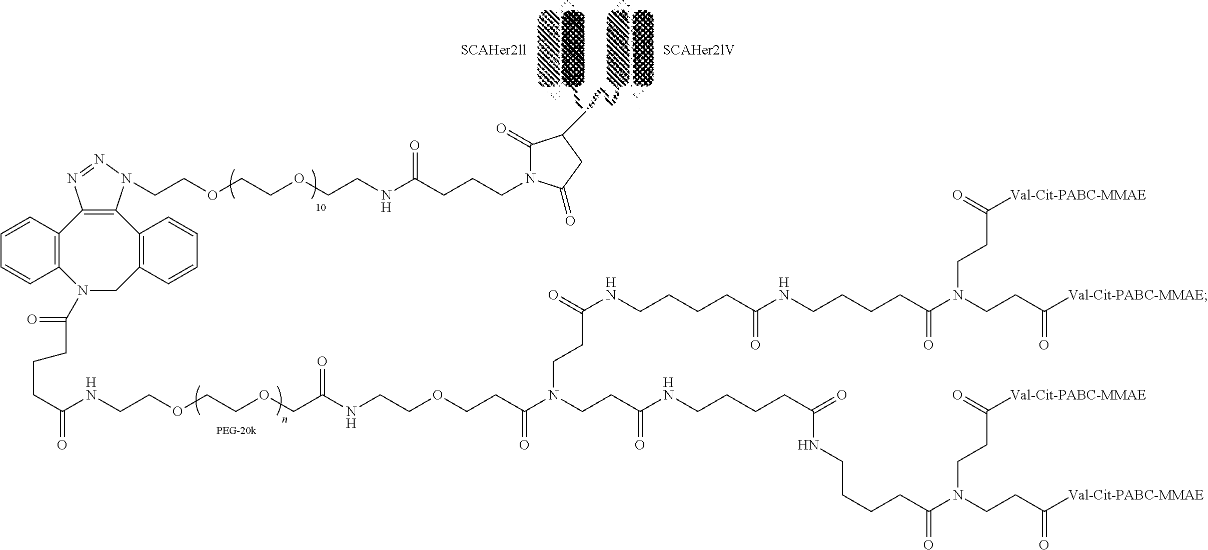

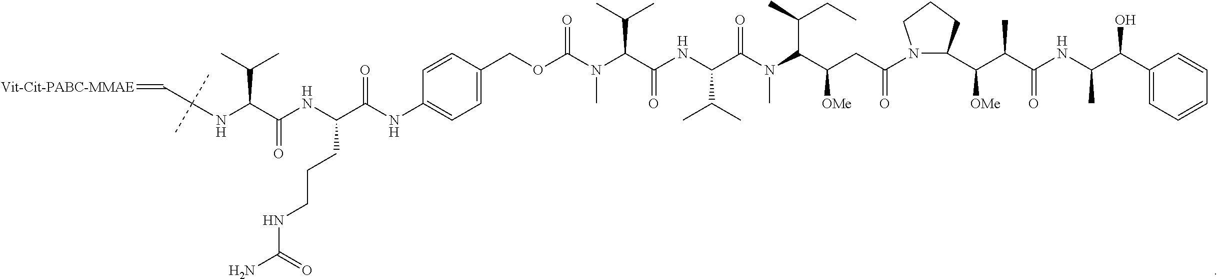

49. (canceled)

50. A pharmaceutical formulation comprising an effective amount of the compound of claim 1 and a pharmaceutically acceptable salt, carrier or excipient.

51. A method for the treatment of a cancer selected from the group consisting of breast cancer, ovarian cancer, prostate cancer, lung cancer, pancreatic cancer, kidney cancer, bladder cancer, stomach cancer, colon cancer, colorectal cancer, salivary gland cancer, thyroid cancer and endometrial cancer, wherein the method comprises administering an effective amount of the compound of claim 1 to a subject.

52. (canceled)


SEQUENCE LISTING (4 sequences)

SEQ ID NO: 1 - protein, 506 aa, Artificial Sequence
  SCAHer2IIxSCAHer2IV
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTGGSGGSGGSGGSGGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGCGSGGSGGSGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGSGGSGGSGGSGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTHHHHHH

SEQ ID NO: 2 - protein, 507 aa, Artificial Sequence
  SCAHer2IV/SCAHer2IV
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGSGGSGGSGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGCGSGGSGGSGGSGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGSGGSGGSGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSHHHHHH

SEQ ID NO: 3 - protein, 508 aa, Artificial Sequence
  SCAHer2IVxSCAHer3
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGSGGSGGSGGSGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGCGSGGSGGSGGSGGQVQLQQWGAGLLKPSETLSLTCAVYGGSFSGYYWSWIRQPPGKGLEWIGEINHSGSTNTNPSLKSRVTISVETSKNQFSLKLSSVTAADTAVYYCARDKWTWYFDLWGRGTLVTVSSGGSGGSGGSGGSGGDIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKHHHHHH

SEQ ID NO: 4 - protein, 501 aa, Artificial Sequence
  SCAc-Met1xSCAc-Met2
DIQMTQSPSSLSASVGDRVTITCSVSSSVSSIYLHWYQQKPGKAPKLLIYSTSNLASGVPSRFSGSGSGTDFTLTISSLQPEDFATYYCIQYSGYPLTFGGGTKVEIKGGSGGSGGSGGSGGQVQLVQSGAEVKKPGASVKVSCKASGYTFTDYYMHWVRQAPGQGLEWMGRVNPNRGGTTYNQKFEGRVTMTTDTSTSTAYMELRSLRSDDTAVYYCARTNWLDYWGQGTTVTVSGCGSGGSGGSGGSGGQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSGGSGGSGGSGGSGGDIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRHHHHHH